(19) United States
(12) Patent Application Publication
(10) Pub. No.: US 2022/0259301 A1
(43) Pub. Date: Aug. 18, 2022

(54) METHODS OF TREATING CROHN'S DISEASE AND ULCERATIVE COLITIS

(71) Applicants: AbbVie Inc., North Chicago, IL (US); AbbVie Deutschland GMBH & Co KG, Wiesbaden (DE)

(72) Inventors: Kori Wallace, Northbrook, IL (US); Anne M. Robinson, Highland, IN (US); Ezequiel Neimark, East Greenwich, RI (US); Alexandra P. Song, Niles, IL (US); Jasmina Kalabic, Frankfurt (DE); Bidan Huang, Vernon Hills, IL (US); Xiaomei Liao, Vernon Hills, IL (US); Graham A. Heap, Boston, MA (US)

(21) Appl. No.: 17/570,010

(22) Filed: Jan. 6, 2022

Related U.S. Application Data
(60) Provisional application No. 63/279,978, filed on Nov. 16, 2021.
(60) Provisional application No. 63/195,554, filed on Jun. 1, 2021.
(60) Provisional application No. 63/134,506, filed on Jan. 6, 2021.
(60) Provisional application No. 63/134,549, filed on Jan. 6, 2021.

(51) Int. Cl. C07K 16/24; A61K 9/00; A61P 1/04

(52) U.S. Cl. CPC C07K 16/244; C07K 2317/24; A61P 1/04; A61K 9/0019

(57) ABSTRACT

The present disclosure relates to methods of treating Crohn's disease or inducing remission of Crohn's disease in a subject. The present disclosure also relates to methods of treating ulcerative colitis or inducing remission of ulcerative colitis in a subject.

### RELATED APPLICATIONS

This application claims the benefit of priority to U.S. Provisional Application No. 63/134,549, filed Jan. 6, 2021; U.S. Provisional Application No. 63/195,554, filed Jun. 1, 2021; U.S. Provisional Application No. 63/279,978, filed Nov. 16, 2021; and U.S. Provisional Application No. 63/134,506, filed Jan. 6, 2021. The entire content of each of these applications is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jan. 6, 2022, is named AVR-71301_ST25.txt, and is 9,705 bytes in size.

### BACKGROUND

Crohn's disease (CD) encompasses a spectrum of clinical and pathological processes manifested by focal asymmetric, transmural, and occasionally granulomatous inflammation that can affect any segment of the gastrointestinal tract and presents with symptoms of fatigue, prolonged diarrhea with or without gross bleeding, abdominal pain, weight loss, and fever (Hanauer et al. (2001) Am. J. Gastroenterol. 96(3):635-43). The disease can affect persons of any age, and its onset is most common in the second and third decades. Females are affected slightly more than males, and the risk for disease is higher in some ethnic groups (Loftus E. V. (2004) Gastroenterology. 126(6):1504-17; Probert et al. (1996) Int. J. Colorectal. Dis. 11(1):25-8). The incidence of CD has steadily increased in developed countries over the past 3 decades (Molodecky et al. (2012) Gastroenterology. 142(1):46-54) with recent estimates varying from 12.7 and 20.2 cases/100,000, and a prevalence of 319 and 322 cases/100,000 in North America and Europe, respectively (Baumgart D. C. (2012) Crohn's disease. Lancet. 380(9853):1590-605). In Asia, the incidence of CD is estimated to be 0.5 to 1.0 cases per 100,000 persons, with a prevalence rate ranging from 3.6 to 7.7 (Leong et al. (2004) Inflamm. Bowel Dis. 10(5):646-51; Lee et al. (2000) J. Gastroenterol. Hepatol. 15(6):622-5).

The exact cause of CD is still unknown, but is hypothesized to be the result of a dysregulated immune system in the context of a genetically susceptible individual. It is thought that a combination of a patient's genetics, microbiome, immune response, and the environment result in an excessive and abnormal immune response in the gut that results in pathology seen in CD (Loftus E. V. (2004) Gastroenterology. 126(6):1504-17).

The aim of medical treatment in CD has been focused on controlling inflammation and reducing symptoms (Lichtenstein et al. (2009) Am. J. Gastroenterol. 104(2):465-83). In addition to improving symptoms, an emerging goal of therapy is to heal the gut mucosa. Resolution of intestinal ulcers, also known as mucosal healing has been associated with positive clinical benefits, including higher rates of clinical remission, fewer hospitalizations, and fewer abdominal surgeries (Froslie et al. (2007) Gastroenterology. 133(2):412-22; Kakkar et al. (2011) Gastroenterol Hepatol. (NY).7(6):374-80). However, improvement of the appearance of the intestinal mucosa may be more difficult to achieve than symptomatic improvement alone.

Conventional pharmaceutical therapies (e.g., corticosteroids, aminosalicylates, thiopurines, methotrexate) are limited, do not always completely abate the inflammatory process, and have significant adverse effects (Hanauer et al. (2001) Am. J. Gastroenterol. 96(3):635-43; Dignass et al. (2010) J. Crohns Colitis. 4(1):28-62). The advent of anti-TNFα agents (e.g., adalimumab) and integrin inhibitors (e.g., vedolizumab) have been shown to achieve clinical remission in patients refractory to conventional therapies (Froslie et al. (2007) Gastroenterology. 133(2):412-22; Kakkar et al. (2011) Gastroenterol Hepatol. (NY).7(6):374-80; Dignass et al. (2010) J. Crohns Colitis. 4(1):28-62; Colombel et al. (2007) Gastroenterology. 132(1):52-65; Sandborn et al. (2013) N. Engl. J. Med. 369(8):711-21).

Despite the benefits of available biologic therapies, many patients do not respond to initial treatment (primary loss of response) or lose treatment over time (secondary loss of response). Regarding anti-TNF agents, approximately 40% of patients will experience primary non-response and secondary non-response occurs in 38% of patients at 6 months and 50% of patients at 1 year (Colombel et al. (2007) Gastroenterology. 132(1):52-65; Targan et al. (1997) N. Engl. J. Med. 337(15):1029-35; Hanauer et al. (2002) Lancet. 359(9317):1541-9; Hanauer et al. (2006) Gastroenterology. 130(2):323-33; Sandborn et al. (2007) N. Engl. J. Med. 357(3):228-38). Additionally, some patients are not candidates for available biologic therapies. Therefore new therapeutic options are needed in order to continue to improve the outcome of patients with CD.

Ulcerative colitis (UC) is one of the two primary forms of idiopathic inflammatory bowel disease (IBD). UC is a chronic, relapsing inflammatory disease of the large intestine characterized by inflammation and ulceration of mainly the mucosal and occasionally submucosal intestinal layers. It is postulated to be caused by an unregulated and exaggerated local immune response to environmental triggers in genetically susceptible individuals (Hanauer S. B. (2004) Nat. Clin. Pract. Gastroenterol. Hepatol. 1:26-31). The highest annual incidence of UC is 24.3 per 100,000 person-years in Europe, 6.3 per 100,000 person-years in Asia and the Middle East, and 19.2 per 100,000 person-years in North America, with a prevalence of 505 cases per 100,000 persons in Europe and 249 cases per 100,000 persons in North America (Molodecky et al. (2012) Gastroenterology 142:46-54). The burden of UC on the healthcare system is profound, accounting for nearly 500,000 physician visits and more than 46,000 hospitalizations per year in the United States (US) alone (Sandler et al. (2002) Gastroenterology 122:1500-1511).

The aim of medical treatment in UC is to control inflammation and reduce symptoms. Available pharmaceutical therapies are limited, do not always completely abate the inflammatory process, and may have significant adverse effects. Therapies for mild to moderate active UC include 5-aminosalicylic acid derivatives and immunosuppressants. Corticosteroids are used in patients with more severe symptoms but are not useful for longer term therapy (Truelove S. C. & Witts L. J. (1959) Br. Med. J. 1:387-394). The frequency and severity of corticosteroid toxicities are significant, including infections, emotional and psychiatric disturbances, skin injury, and metabolic bone disease. Corticosteroids are not effective for the maintenance of remission and the UC practice guidelines from the American College of Gastroenterology and the European Crohn's and Colitis Organization recommend against chronic steroid treatment (The European evidence-based consensus on diagnosis and management of ulcerative colitis. Part 2: current management).

Patients with moderate to severe symptoms may derive some benefits from immunomodulatory agents (azathioprine [AZA], mercaptopurine [6-MP], or methotrexate [MTX]), however, up to 17% of patients on these agents experience adverse events severe enough to necessitate drug withdrawal (Chaparro et al. (2013) Inflamm. Bowel Dis. 19:1404-1410). Adverse events (AEs) include idiosyncratic flu like reactions, bone marrow suppression, hepatotoxicity, pancreatitis, infections and malignancies (Kombluth et al. (2010) Am. J. Gastroenterol. 105:501-523; Beaugerie et al. (2009) Lancet. 374:1617-1625). Despite these therapies, approximately 15% of ulcerative colitis patients experience a severe clinical course, and 30% of these patients require removal of the colon/rectum, to eliminate the source of the inflammatory process. This procedure is accompanied by significant morbidity (Turner et al. (2007) Clin. Gastroenterol. Hepatol. 5:103-110).

Biological agents targeting specific immunological pathways have been evaluated for their therapeutic effect in treating patients with UC. Anti-tumor necrosis factor (TNF) agents were the first biologics to be used for IBD. Infliximab, adalimumab, and golimumab are successfully being used for the treatment of UC. Recently, vedolizumab, an anti-adhesion therapy, has been approved for the treatment of UC by the US Food and Drug Administration (FDA) and the European Medicines Agency (EMA), and clinical development is ongoing in Japan. Anti-TNF therapies are an effective treatment for patients who are steroid refractory or steroid dependent, who had inadequate response to a thiopurine, or who are intolerant to these medications. Potential risks with anti-TNF therapies include infusion or injection site reactions, serious infections, lymphoma, heart failure, lupus-like syndromes, and demyelinating conditions (Sandborn et al. (2010) Dig. Dis. 28:536-542).

Despite the beneficial results achieved with the available biologic agents, only 17% to 45% of patients who receive them are able to achieve clinical remission (Rutgeerts, et al. (2005) N. Engl. J. Med. 353:2462-2476). Thus, there remains a clear medical need for additional therapeutic options in UC for patients with inadequate response to or intolerance to conventional therapies and biologic therapies.

### SUMMARY

The methods described herein provide an effective and safe dosing regimen for administering risankizumab (also referred to in the United States as risankizumab-rzaa), to treat Crohn's disease or induce remission of Crohn's disease in a subject. The present disclosure relates to methods of treating Crohn's disease or inducing remission of Crohn's disease in a subject comprising administering to the subject at least one induction dose of about 600 mg or about 1200 mg of risankizumab.

Accordingly, in one aspect, the present disclosure relates to a method of treating Crohn's disease in a subject comprising administering to the subject at least one induction dose of risankizumab, wherein the induction dose comprises about 600 mg or about 1200 mg of risankizumab. In one embodiment, the method further comprises administering to the subject at least one maintenance dose of about 180 mg or about 360 mg of risankizumab.

In another aspect, the present disclosure relates to a method of inducing remission of Crohn's disease and/or an endoscopic response in a subject comprising administering to the subject at least one induction dose of risankizumab, wherein the induction dose comprises about 600 mg or about 1200 mg of risankizumab. In one embodiment, the method further comprises administering to the subject at least one maintenance dose of about 180 mg or about 360 mg of risankizumab.

Additionally, provided herein are methods of treating ulcerative colitis or inducing remission of ulcerative colitis in a subject comprising administering to the subject at least one induction dose of about 600 mg, about 1200 mg or about 1800 mg of risankizumab. The methods described herein provide an effective and safe dosing regimen for administering risankizumab (also referred to in the United States as risankizumab-rzaa), to treat ulcerative colitis or induce remission of ulcerative colitis in a subject.

Accordingly, in one aspect, the present disclosure relates to a method of treating ulcerative colitis in a subject comprising administering to the subject at least one induction dose of risankizumab, wherein the induction dose comprises about 600 mg, about 1200 mg or about 1800 mg of risankizumab.

In another aspect, the present disclosure relates to a method of inducing remission of ulcerative colitis in a subject comprising administering to the subject at least one induction dose of risankizumab, wherein the induction dose comprises about 600 mg, about 1200 mg or about 1800 mg of risankizumab.

### BRIEF DESCRIPTION OF THE DRAWINGS

FIG. 1 shows the study design of M15-993 and M15-989. *Double-blinded treatment was administered in three groups: PBO, RZB 200 mg IV Q4W, and RZB 600 mg IV Q4W. †At the end of week 12, patients who did not achieve deep remission (clinical remission and endoscopic remission) received OL RZB 600 mg IV Q4W; those who achieved deep remission entered washout in Period 2. Subjects who achieved clinical remission entered Period 3 and received OL 180 mg SC Q8W. Patients who completed M15-993 in clinical response/remission were eligible for the OLE M15-989. ‡Patients rolled over to the ongoing phase 3 OLE (M16-000 sub-study 3) between weeks 128 and 184 of M15-989. IV=intravenous; OL=open label; OLE=OL extension; PBO=placebo; Q4/8W=every 4 or 8 weeks; RZB=risankizumab; SC=subcutaneous.

FIG. 2 shows the patient disposition of OLE M15-989. *Including patients with clinical remission and/or response at screening of M15-989. ^{†}In these patients, clinical response and/or remission were reassessed at week 12; if clinical response and/or remission were achieved, patients were switched to risankizumab 180 mg SC Q8W from visit 5. ^{‡}Primary reasons for discontinuation. ^{§} Other reasons are as reported by the investigator and have not been grouped to avoid inaccuracy in reporting. AE=adverse event; CD=Crohn's disease; IV=intravenous; OLE=open-label extension; Q4/8W=every 4/8 weeks; SC, subcutaneous.

FIG. 3 shows the risankizumab trough plasma concentration overtime in all patients in M15-989. Analysis set included all patients with available pharmacokinetics and ADA data. Patients rolled over to the ongoing phase 3 OLE (M16-000 sub-study 3) between weeks 128 and 184, and as a result had different last visit time points. ADA=anti-drug antibody; OLE=open-label extension; SC=subcutaneous.

FIGS. 4A-4D show proportions of patients achieving clinical remission, CDEIS remission, IBDQ response, and IBDQ remission over time (intent-to-treat analysis set) in M15-989. NRI for missing data were presented up to week 128, as from that point onwards patients could roll over to the ongoing phase 3 OLE (M16-000 sub-study 3). Only observed cases were presented beyond week 128. n denotes the number of responders and N denotes the number of patients at risk. CDAI=Crohn's Disease Activity Index; CDEIS=Crohn's Disease Endoscopic Index of Severity; IBDQ=Inflammatory Bowel Disease Questionnaire; NRI=non-responder imputation; OLE=open-label extension.

FIGS. 5A-5C show median change (IQR) from baseline in hs-CRP (intent-to-treat analysis set), faecal calprotectin (intent-to-treat analysis set), and serum albumin (safety analysis set) over time in M15-989. Baseline defined as last measurement prior to first dose of study drug in M15-993; week 0 defined as first dose of study drug in M15-989. Patients rolled over to the ongoing phase 3 OLE (M16-000 sub-study 3) between weeks 128 and 184, and as a result had different last visit time points. hs-CRP=high-sensitivity C-reactive protein; IQR=interquartile range; OLE=open-label extension.

FIG. 6 shows the study M15-991 study schematic.

FIG. 7 shows the study M16-006 study schematic.

FIG. 8 shows the study M16-000 sub-study 1 & 2 schematic.

FIG. 9 shows the study M16-000 sub-study 3 schematic.

FIG. 10 shows clinical remission (CDAI) rates overtime in M16-000 sub-study 1.

FIG. 11 shows clinical remission (SS/APS) rates over time in M16-000 sub-study 1.

FIG. 12 shows clinical remission (CDAI) rates overtime in study M16-006 following first interim database lock; where CDAI refers to the Crohn's disease activity index.

FIG. 13 shows clinical remission (PRO2) rates overtime in study M16-006 following first interim database lock; where PRO2 refers to Patient Reported Outcome related to stool frequency and abdominal pain (SF/APS) which are components of CDAI.

FIG. 14 shows clinical remission (CDAI) rates overtime in study M15-991 following first interim database lock.

FIG. 15 shows clinical remission (PRO2, SF/APS) rates overtime in study M15-991 following first interim database lock.

FIG. 16 shows clinical remission by CDAI and SF/APS criteria at week 12 (NRI-C) of MOTIVATE and ADVANCE studies by baseline steroid use (ITT1A population). The ITT1A population includes randomized subjects who received at least 1 dose of study drug during the 12-week induction period and had baseline eligible SES-CD of ≥6 (≥4 for isolated ileal disease). Clinical remission by CDAI: CDAI <150. Clinical remission by SF/APS: average daily SF ≤2.8 and not worse than baseline and average daily APS ≤1 and not worse than baseline. APS, abdominal pain score; CD, Crohn's disease; CDAI, CD Activity Index; ITT, intent-to-treat; NRI-C, non-responder imputation incorporating multiple imputation to handle missing data due to COVID-19; PBO, placebo; RZB, risankizumab; SES-CD, Simple Endoscopic Score for CD; SF, stool frequency.

FIG. 17A shows achievement of discontinuation of corticosteroid use (in patients using steroids at induction baseline). FIG. 17B shows week 52 steroid-free clinical remission at Week 52. FIG. 17C shows week 52 steroid-free endoscopic outcomes (NRI-C) during maintenance treatment in the ITT1A population. The ITT1A population includes randomized subjects who received at least 1 dose of study drug during the 12-week induction period and had baseline eligible SES-CD of ≥6 (≥4 for isolated ileal disease). Clinical remission by CDAI: CDAI <150. Clinical remission by SF/APS: average daily SF ≤2.8 and not worse than baseline and average daily APS ≤1 and not worse than baseline of induction. Endoscopic remission: SES-CD ≤4 and ≥2-point reduction vs baseline of the induction study and no subscore >1 in any individual variable. Endoscopic response: >50% decrease from baseline in SES-CD (or ≥2-point reduction from baseline for patients with isolated ileal disease and baseline SES-CD of 4). APS, abdominal pain score; CD, Crohn's disease; CDAI, CD Activity Index; NRI-C, non-responder imputation incorporating multiple imputation to handle missing data due to COVID 19; PBO, placebo; RZB, risankizumab; SES-CD, Simple Endoscopic Score for CD; SF, stool frequency.

FIG. 18 is a schematic diagram showing Induction Period 1 of Study M16-067 Sub-Study 4* (Phase 2b). DC=discontinue; IR=subjects with inadequate clinical response; IV=intravenous; R=subjects with clinical response; SS-4=Sub-Study 4. *DBL for Study M16-067 Sub-Study 4 occurred after first randomized 240 subjects completed 12 weeks of assessment. After 240 subjects had been enrolled and before the dose had been selected for Sub-Study 5, additional subjects continued to be enrolled into Sub-Study 4, where they were assigned to open label risankizumab 1800 mg IV Wks 0, 4 and 8 dosing group.

FIG. 19 is a schematic diagram showing Induction Period 2 of Study M16-067 (Sub-Study 4 and Sub-Study 5). DC=discontinue. RZB=risankizumab; IR=subjects with inadequate response; IV=intravenous; Plb=placebo; R=subjects with clinical response; RR=re-randomize; SC=subcutaneous. *Subjects who enter from M16-067 Sub-Study 4 receive risankizumab 1800 mg IV. Subjects who enter from M16-067 Sub-Study 5 receive 1200 mg risankizumab IV.

FIG. 20 is a schematic diagram showing Induction Period 1 of Study M16-067 Sub-Study 5 (Phase 3). DC=discontinue; IR=subjects with inadequate clinical response; R=subjects with clinical response; SS-5=Sub study 2.

FIG. 21 is a schematic diagram showing Study M16-066 Sub-Study 4 & 2. RZB: isankizumab; SC=subcutaneous; Q8W=every 8 weeks; IV=intravenous; TDM=therapeutic drug monitoring; CA=clinical assessment; OLE=Open-label extension; R=subjects with clinical response.

FIG. 22 is a schematic diagram showing Study M16-066 Sub-Study 6. RZB: isankizumab; SC: subcutaneous; Q8W: every 8 weeks; OLE: Open-Label extension. End of study defined as approximately 300 weeks or study discontinuation, whichever is earlier.

FIG. 23 shows mean change from baseline in Partial Adapted Mayo score over time (ITT1A, MMRM) with 90% confidence intervals. Vertical bars represent 90% confidence interval for LS Mean. ***, **, * indicate statistical significance at 0.001, 0.01, 0.05 and 0.1 (two-sided) level, respectively.

FIG. 24 shows generally dose-proportional increase in exposure.

### DETAILED DESCRIPTION

The present disclosure describes the unexpected discovery that administration to a human subject afflicted with moderately and severely active Crohn's disease of at least one induction dose of about 600 or about 1200 mg of risankizumab induces robust clinical response and remission.

Additionally, the present disclosure describes the unexpected discovery that administration to a human subject afflicted with moderately and severely active ulcerative colitis of at least one induction dose of about 600 mg, about 1200 mg or about 1800 mg of risankizumab induces robust clinical response and remission.

As disclosed herein, the present disclosure relates to the following embodiments.

Crohn's Disease

Embodiment 1. A method for treating Crohn's disease (CD), inducing remission of CD, and/or inducing an endoscopic response comprising administering to a subject at least one induction dose of risankizumab, wherein the induction dose is about 600 mg or about 1200 mg of risankizumab.

Embodiment 2. The method of embodiment 1, wherein the subject is a patient afflicted with moderately to severely active Crohn's disease.

Embodiment 3. The method of embodiment 1 or 2, wherein the subject has:

(1) average daily stool frequency (SF) ≥4 and/or average daily abdominal pain (AP) score ≥2; plus

(2) Simple Endoscopic Score for CD (SES-CD) ≥3;

before the administration of the at least one induction dose of risankizumab. Alternatively, the subject has:

(1) average daily stool frequency (SF) ≥4 and/or average daily abdominal pain (AP) score ≥2; plus

(2) Simple Endoscopic Score for CD (SES-CD) ≥6 or ≥4 for isolated ileal disease;

before the administration of the at least one induction dose of risankizumab.

Embodiment 4. The method of any one of embodiments 1-3, wherein the subject has Crohn's disease activity index (CDAI) score 220-450 before the administration of the at least one induction dose of risankizumab.

Embodiment 5. The method of any one of embodiments 1-4, wherein the subject has intolerance or an inadequate response to one or more anti-TNF or anti-integrin biologics for CD.

Embodiment 6. The method of embodiment 5, wherein the anti-TNF or anti-integrin biologic for CD comprises infliximab, adalimumab, certolizumab, vedolizumab, ustekinumab and/or natalizumab.

Embodiment 7. The method of any one of embodiments 1-4, wherein the subject has an inadequate response or intolerance to one or more conventional therapies selected from the group consisting of aminosalicylates, oral locally acting steroids, systemic corticosteroids, and immunomodulators.

Embodiment 8. The method of any one of embodiments 1-7, wherein three induction doses are administered to the subject.

Embodiment 9. The method of any one of embodiments 1-8, wherein three induction doses are administered at 4 week intervals.

Embodiment 10. The method of any one of embodiments 1-9, wherein the induction dose is administered by intravenous infusion.

Embodiment 11. The method of any one of embodiments 1-10, wherein the induction dose is about 600 mg of risankizumab.

Embodiment 12. The method of any one of embodiments 1-10, wherein the induction dose is about 1200 mg of risankizumab.

Embodiment 13. The method of any one of embodiments 1-12, wherein the subject achieves one or more endpoints selected from the group consisting of:

(1) clinical remission: average daily SF ≤2.8 and not worse than Baseline AND average daily AP score s 1 and not worse than Baseline;

(2) enhanced clinical response: ≥60% decrease in average daily SF and/or ≥35% decrease in average daily AP score and both not worse than Baseline, and/or clinical remission;

(3) clinical response: ≥30% decrease in average daily SF and/or ≥30% decrease in average daily AP score;

(4) endoscopic response: decrease in SES-CD >50% from Baseline, or for subjects with isolated ileal disease and a Baseline SES-CD of 4, at least a 2 point reduction from Baseline;

(5) ulcer-free endoscopy: SES-CD ulcerated surface subscore of 0 in subjects with SES-CD ulcerated surface subscore ≥1 at Baseline;

(6) endoscopic remission: SES-CD ≤4 and at least a 2 point reduction versus baseline and no subscore greater than 1 in any individual variable;

(7) CDAI clinical response: reduction of CDAI ≥100 points from baseline;

(8) CDAI clinical remission: CDAI <150;

(9) SF remission: average daily SF ≤2.8 and not worse than baseline; and

(10) AP remission: average daily AP score ≤1 and not worse than baseline.

Embodiment 14. The method of any one of claims 1-12, wherein when a population of patients is treated, 30-60% of the population achieve clinical remission as measured by CDAI or SF/APS.

Embodiment 15. The method of any one of embodiments 1-14, wherein the method further comprises

(a) administering to the subject a first maintenance dose of risankizumab after the last induction dose is administered, and

(b) administering at least one additional maintenance dose to the subject after the first maintenance dose is administered.

Embodiment 16. The method of embodiment 15, wherein the first maintenance dose is administered 4 weeks after the last induction dose is administered.

Embodiment 17. The method of embodiment 15 or 16, wherein the at least one additional maintenance dose is administered 8 weeks after the first maintenance dose is administered.

Embodiment 18. The method of any one of embodiments 15-17, wherein more than one additional maintenance doses are administered after the first maintenance dose.

Embodiment 19. The method of any one of embodiments 15-18, wherein each additional maintenance dose is administered at 8 weeks intervals.

Embodiment 20. The method of any one of embodiments 15-19, wherein the first maintenance dose is about 180 mg or about 360 mg of risankizumab.

Embodiment 21. The method of any one of embodiments 15-20, wherein the additional maintenance dose is about 180 mg or about 360 mg of risankizumab.

Embodiment 22. The method of any one of embodiments 15-21, wherein both the first maintenance dose and the additional maintenance dose are about 180 mg or 360 mg of risankizumab.

Embodiment 23. The method of any one of embodiments 15-22, wherein both the first maintenance dose and the additional maintenance dose are about 360 mg of risankizumab.

Embodiment 24. The method of any one of embodiments 15-22, wherein both the first maintenance dose and the additional maintenance dose are about 180 mg of risankizumab.

Embodiment 25. The method of any one of embodiments 15-22, wherein the subject receives three induction doses of 600 mg risankizumab at weeks 0, 4, and 8; and further receives the first maintenance dose of 360 mg risankizumab at week 12, and one or more additional maintenance doses of 360 mg risankizumab at 8-week intervals after the first maintenance dose.

Embodiment 26. The method of any one of embodiments 15-22, wherein the subject receives three induction doses of 600 mg risankizumab at weeks 0, 4, and 8; and further receives the first maintenance dose of 180 mg risankizumab at week 12, and one or more additional maintenance doses of 180 mg risankizumab at 8-week intervals after the first maintenance dose.

Embodiment 27. The method of any one of embodiments 15-22, wherein the subject receives three induction doses of 1200 mg risankizumab at weeks 0, 4, and 8; and further receives the first maintenance dose of 360 mg risankizumab at week 12, and one or more additional maintenance doses of 360 mg risankizumab at 8-week intervals after the first maintenance dose.

Embodiment 28. The method of any one of embodiments 15-22, wherein the subject receives three induction doses of 1200 mg risankizumab at weeks 0, 4, and 8; and further receives the first maintenance dose of 180 mg risankizumab at week 12, and one or more additional maintenance doses of 180 mg risankizumab at 8-week intervals after the first maintenance dose.

Embodiment 29. The method of any one of embodiments 15-28, wherein the first maintenance dose and/or the additional maintenance dose are administered by subcutaneous injection.

Embodiment 30. The method of any one of embodiments 15-29, wherein the subject maintains one or more endpoints selected from the group consisting of:

(1) clinical remission: average daily SF ≤2.8 and not worse than Baseline AND average daily AP score s 1 and not worse than Baseline;

(2) enhanced clinical response: ≥60% decrease in average daily SF and/or ≥35% decrease in average daily AP score and both not worse than Baseline, and/or clinical remission;

(3) clinical response: ≥30% decrease in average daily SF and/or ≥30% decrease in average daily AP score;

(4) endoscopic response: decrease in SES-CD >50% from Baseline, or for subjects with isolated ileal disease and a Baseline SES-CD of 4, at least a 2 point reduction from Baseline;

(5) ulcer-free endoscopy: SES-CD ulcerated surface subscore of 0 in subjects with SES-CD ulcerated surface subscore ≤1 at Baseline;

(6) endoscopic remission: SES-CD ≥4 and at least a 2 point reduction versus baseline and no subscore greater than 1 in any individual variable;

(7) Deep remission: clinical remission and endoscopic remission;

(8) CDAI clinical response: reduction of CDAI ≥100 points from baseline; and

(9) CDAI clinical remission: CDAI <150.

Embodiment 31. The method of any one of embodiments 1-30, wherein the method is for inducing remission of CD.

Embodiment 32. The method of any one of embodiments 1-30, wherein the method is for treating CD.

Embodiment 33. A method for inducing remission of moderately to severely active Crohn's disease (CD) in an adult patient suffering from CD, comprising:
(a) intravenously administering to the patient three 600 mg induction doses of risankizumab at four week intervals; and
(b) subcutaneously administering to the patient a first maintenance dose of risankizumab four weeks after the third induction dose is administered; and
(c) subcutaneously administering at least one additional maintenance dose is administered at eight week intervals,
wherein the patient achieves remission of CD at 4 week, 8 weeks, 12 weeks, 24 weeks, or 52 weeks following the administration of the first induction dose.

Embodiment 34. The method of embodiment 33, wherein the patient has:
(1) average daily stool frequency (SF) score ≥4 and/or average daily abdominal pain (AP) score ≥2; or
(2) Simple Endoscopic Score for CD (SES-CD) ≥3, before the administration of the first induction dose of risankizumab.

Embodiment 35. The method of embodiments 33 or 34, wherein the patient has Crohn's disease activity index (CDAI) score 220-450 before the administration of the first induction dose of risankizumab.

Embodiment 36. The method of embodiments 33-35, wherein the patient achieves, at 4 weeks, 8 weeks, 12 weeks, 24 weeks, or 52 weeks following the administration of the first induction dose, one or more endpoints selected from the group consisting of:
(1) clinical remission: average daily SF [symbol] 2.8 and not worse than Baseline, and average daily AP score [symbol] 1 and not worse than Baseline;
(2) enhanced clinical response: [symbol] 60% decrease in average daily SF and/or [symbol] 35% decrease in average daily AP score and both not worse than Baseline, and/or clinical remission;
(3) clinical response: [symbol] 30% decrease in average daily SF and/or [symbol] 30% decrease in average daily AP score;
(4) endoscopic response: decrease in SES-CD >50% from Baseline, or for patients with isolated ileal disease and a Baseline SES-CD of 4, at least a 2 point reduction from Baseline;
(5) ulcer-free endoscopy: SES-CD ulcerated surface subscore of 0 in patients with SES-CD ulcerated surface subscore [symbol] 1 at Baseline;
(6) endoscopic remission: SES-CD [symbol] 4 and at least a 2 point reduction versus baseline and no subscore greater than 1 in any individual variable;
(7) Deep remission: clinical remission and endoscopic remission;
(8) CDAI clinical response: reduction of CDAI [symbol] 100 points from baseline;
(9) CDAI clinical remission: CDAI <150;
(10) steroid-free clinical remission at week 52 by CDAI: CDAI <150;
(11) steroid-free clinical remission at week 52 by SF/APS: average daily SF [symbol] 0.8 and not worse than baseline and average daily APS [symbol] 1 and not worse than baseline;
(12) steroid-free endoscopic remission at week 52: SES-CD [symbol] 4 and [symbol] 2-point reduction vs baseline and no subscore >1 in any individual variable; and
(13) steroid-free endoscopic response at week 52: >50% decrease from baseline in SES-CD (or [symbol] 2-point reduction from baseline for patients with isolated ileal disease and baseline SES-CD of 4).

Embodiment 37. The method of embodiments 33-36, wherein the patient has intolerance or an inadequate response to one or more of anti-TNF, anti-integrin, or anti-p40 biologics for CD.

Embodiment 38. The method of embodiment 37, wherein the anti-TNF or anti-integrin biologic for CD comprises infliximab, adalimumab, certolizumab, vedolizumab, ustekinumab and/or natalizumab.

Embodiment 39. The method of embodiments 33-38, wherein the patient has an inadequate response or intolerance to one or more conventional therapies selected from the group consisting of aminosalicylates, oral locally acting steroids, systemic corticosteroids, and immunomodulators.

Embodiment 40. The method of embodiments 33-39, wherein both the first maintenance dose and the additional maintenance dose are 180 mg of risankizumab.

Embodiment 41. The method of embodiments 33-39, wherein both the first maintenance dose and the additional maintenance dose are 360 mg of risankizumab.

Embodiment 41A. A method for treating or inducing remission of moderately to severely active Crohn's disease (CD) in an adult patient suffering from CD, comprising:
(a) intravenously administering to the patient three 600 mg induction doses of risankizumab at four week intervals; and
(b) subcutaneously administering to the patient a first maintenance dose of risankizumab four weeks after the third induction dose is administered; and
(c) subcutaneously administering at least one additional maintenance dose is administered at eight week intervals,
wherein when the method is compared to treatment with placebo in a population of human subjects suffering from moderately to severely active CD, a statistically significantly higher percentage of the human subjects treated with the method achieves remission 4 weeks, 8 weeks, 12 weeks, 24 weeks, or 52 weeks following the administration of the first induction dose.

Ulcerative Colitis

Embodiment 42. A method of treating ulcerative colitis (UC) or inducing remission of UC in a subject comprising administering to the subject at least one induction dose of risankizumab, wherein the induction dose comprises about 600 mg, about 1200 mg or about 1800 mg of risankizumab.

Embodiment 43. The method of embodiment 42, wherein the subject is a patient afflicted with moderately to severely active ulcerative colitis.

Embodiment 44. The method of embodiment 42 or 43, wherein the subject has a Adapted Mayo score of 5 to 9 points and an endoscopic subscore of 2 to 3 before the administration of the at least one induction dose of risankizumab.

Embodiment 45. The method of any one of embodiments 42-44, wherein the subject has intolerance or an inadequate response to one or more biologic therapies for ulcerative colitis.

Embodiment 46. The method of any one of embodiments 42-45, wherein the biologic therapies comprise infliximab, adalimumab, golimumab, or vedolizumab.

Embodiment 47. The method of any one of embodiments 42-46, wherein three induction doses are administered to the subject.

Embodiment 48. The method of any one of embodiments 42-47, wherein the three induction doses are administered at 4 weeks intervals.

Embodiment 49. The method of any one of embodiments 42-48, wherein the induction dose is administered by intravenous infusion.

Embodiment 50. The method of any one of embodiments 42-49, wherein the induction dose comprises about 1200 mg of isankizumab.

Embodiment 51. The method of any one of embodiments 42-50, wherein the induction dose comprises about 1800 mg of isankizumab.

Embodiment 52. The method of any one of embodiments 42-51, wherein the subject achieves one or more of the endpoints selected from the group consisting of:

(1) clinical remission per Adapted Mayo (defined by a stool frequency subscore (SFS) ≤1 and not greater than baseline, a rectal bleeding subscore (RBS)=0, and an endoscopic subscore ≤1);

(2) clinical response per Adapted Mayo (defined by a decrease from baseline in the Adapted Mayo score ≥2 points and ≥30% from baseline, plus a decrease in a rectal bleeding subscore (RBS) ≥1 or an absolute RBS ≤1);

(3) clinical response per partial Adapted Mayo (defined by a decrease from baseline in the Adapted Mayo score ≥1 points and ≥30% from baseline, plus a decrease in a RBS ≥1 or an absolute RBS ≤1);

(4) clinical remission per Full Mayo (defined by Full Mayo score ≤2 with no subscore >1);

(5) endoscopic improvement (defined by an endoscopy subscore of 0 or 1);

(6) endoscopic remission (defined by an endoscopy subscore=0);

(7) histologic remission (defined by a Geboes score <2); and

(8) mucosal healing (defined by the endoscopic and histologic remission).

Embodiment 53. The method of any one of embodiments 42-51, wherein when a population of patients is treated, 8-12% of the population achieve clinical remission per Adapted Mayo.

Embodiment 54. The method of any one of embodiments 52-51, wherein when a population of patients is treated, 14%-16% of the population achieve endoscopic improvement.

Embodiment 55. The method of any one of embodiments 42-51, wherein when a population of patients is treated, 5%-9% of the population achieve clinical remission per Full Mayo score.

Embodiment 56. The method of any one of embodiments 42-51, wherein when a population of patients is treated, 47%-54% of the population achieve Clinical Response per Adapted Mayo Score.

Embodiment 57. The method of any one of embodiments 42-51, wherein when a population of patients is treated, 37%-46% of the population achieve Clinical Response per Partial Adapted Mayo Score.

Embodiment 58. The method of any one of embodiments 42-51, wherein when a population of patients is treated, 4%-7% of the population achieve Endoscopic Remission.

Embodiment 59. The method of any one of embodiments 42-51, wherein when a population of patients is treated, 4%-5% of the population achieve change from Baseline in Inflammatory Bowel Disease Questionnaire (IBDQ).

Embodiment 60. The method of any one of embodiments 42-59, wherein the method further comprises

(a) administering to the subject a first maintenance dose of risankizumab after the last induction dose is administered, and

(b) administering at least one additional maintenance dose to the subject after the first maintenance dose is administered.

Embodiment 61. The method of embodiment 60, wherein the first maintenance dose is administered 4 weeks after the last induction dose is administered.

Embodiment 62. The method of embodiment 60 or 61, wherein the at least one additional maintenance dose is administered 8 weeks after the first maintenance dose is administered.

Embodiment 63. The method of any one of embodiments 60-62, wherein more than one additional maintenance doses are administered after the first maintenance dose.

Embodiment 64. The method of any one of embodiments 60-63, wherein each of the additional maintenance doses is administered at an 8-week interval.

Embodiment 65. The method of any one of embodiments 60-64, wherein the first maintenance dose comprises about 180 mg or about 360 mg of risankizumab.

Embodiment 66. The method of any one of embodiments 60-65, wherein the additional maintenance dose comprises about 180 mg or about 360 mg of risankizumab.

Embodiment 67. The method of any one of embodiments 60-66, wherein both the first maintenance dose and the additional maintenance dose are about 180 mg or 360 mg of risankizumab.

Embodiment 68. The method of any one of embodiments 60-67, wherein both the first maintenance dose and the additional maintenance dose are about 180 mg of risankizumab.

Embodiment 69. The method of any one of embodiments 60-67, wherein both the first maintenance dose and the additional maintenance dose are about 360 mg of risankizumab.

Embodiment 70. The method of any one of embodiments 60-69, wherein the first maintenance dose and/or the additional maintenance dose are administered by subcutaneous injection.

Embodiment 71. The method of any one of embodiments 60-70, wherein the subject receives three induction doses of about 1200 mg risankizumab at weeks 0, 4, and 8; and further receives the first maintenance dose of about 180 mg risankizumab at week 12, and one or more additional maintenance doses of about 180 mg risankizumab at 8-week intervals after the first maintenance dose.

Embodiment 72. The method of any one of embodiments 60-70, wherein the subject receives three induction doses of about 1800 mg risankizumab at weeks 0, 4, and 8; and further receives the first maintenance dose of about 180 mg risankizumab at week 12, and one or more additional maintenance doses of about 180 mg risankizumab at 8-week intervals after the first maintenance dose.

Embodiment 73. The method of any one of embodiments 60-70, wherein the subject receives three induction doses of about 1200 mg risankizumab at weeks 0, 4, and 8; and further receives the first maintenance dose of about 360 mg risankizumab at week 12, and one or more additional maintenance doses of about 360 mg risankizumab at 8-week intervals after the first maintenance dose.

Embodiment 74. The method of any one of embodiments 60-70, wherein the subject receives three induction doses of about 1800 mg risankizumab at weeks 0, 4, and 8; and further receives the first maintenance dose of about 360 mg risankizumab at week 12, and one or more additional maintenance doses of about 360 mg risankizumab at 8-week intervals after the first maintenance dose.

Embodiment 75. The method of any one of embodiments 60-74, wherein the subject maintains one or more of the endpoints selected from the group consisting of:

(1) the clinical remission per Adapted Mayo;

(2) the clinical response per Adapted Mayo;

(3) the clinical response per partial Adapted Mayo;

(4) the clinical remission per Full Mayo

(5) the endoscopic improvement;

(6) the endoscopic remission;

(7) the histologic remission; and

(8) the mucosal healing.

Embodiment 76. The method of any one of embodiments 42-75, wherein the method is for inducing remission of UC.

Embodiment 77. The method of any one of embodiments 42-75, wherein the method is for treating UC.

Embodiment 78. A method for inducing remission of moderately to severely active ulcerative colitis (UC) in an adult patient suffering from UC, comprising intravenously administering to the patient three induction doses of risankizumab at four week intervals, wherein the induction doses each comprises 600 mg, 1200 mg, or 1800 mg of risankizumab, and further wherein the patient achieves remission of the ulcerative colitis at 4 weeks, 8 weeks, or 12 weeks following the administration of the first induction dose.

Embodiment 79. The method of embodiment 78, wherein the induction dose is 600 mg.

Embodiment 80. The method of embodiment 78, wherein the induction dose is 1200 mg.

Embodiment 81. The method of embodiment 78, wherein the induction dose is 1800 mg.

Embodiment 82. The method of any one of embodiments 78-81, wherein the patient has an Adapted Mayo score of 5 to 9 points and an endoscopic subscore of 2 to 3 before the administration of the at least one induction dose of isankizumab.

Embodiment 83. The method of any one of embodiments 78-82, wherein the patient achieves, at 4 weeks, 8 weeks, or 12 weeks following the administration of the first induction dose, one or more of the endpoints selected from the group consisting of:
(1) clinical remission per Adapted Mayo: a stool frequency subscore (SFS) ≤1 and not greater than baseline, a rectal bleeding subscore (RBS)=0, and an endoscopic subscore ≤1;
(2) clinical response per Adapted Mayo: a decrease from baseline in the Adapted Mayo score ≥2 points and ≥30% from baseline, plus a decrease in a rectal bleeding subscore (RBS) ≥1 or an absolute RBS ≤1;
(3) clinical response per partial Adapted Mayo: a decrease from baseline in the Adapted Mayo score ≥1 points and ≥30% from baseline, plus a decrease in a RBS ≥1 or an absolute RBS ≤1;
(4) clinical remission per Full Mayo: Full Mayo score s 2 with no subscore >1;
(5) endoscopic improvement: an endoscopy subscore of 0 or 1;
(6) endoscopic remission: an endoscopy subscore=0;
(7) histologic remission: a Geboes score <2; and
(8) mucosal healing: endoscopic and histologic remission.

Embodiment 84. The method of any one of embodiments 78-83, wherein the patient has intolerance or an inadequate response to one or more biologic therapies for ulcerative colitis.

Embodiment 85. The method of embodiment 84, wherein the biologic therapies comprise infliximab, adalimumab, golimumab, or vedolizumab.

Embodiment 86. The method of any one of embodiments 78-85, further comprising
(a) subcutaneously administering to the patient a first maintenance dose of risankizumab four weeks after the third induction dose is administered, and
(b) subcutaneously administering additional maintenance doses to the patient at eight week intervals after the first maintenance dose is administered, where both the first maintenance dose and the additional maintenance doses each comprises 180 mg or 360 mg of risankizumab.

Embodiment 87. The method of embodiment 86, wherein both the first maintenance dose and the additional maintenance dose are 180 mg of risankizumab.

Embodiment 88. The method of embodiment 87, wherein both the first maintenance dose and the additional maintenance dose are 360 mg of risankizumab.

Embodiment 89. A method for treating or inducing remission of moderately to severely active ulcerative colitis (UC) in an adult patient suffering from UC, comprising intravenously administering to the patient first, second, and third induction doses of risankizumab at four week intervals, wherein each of the induction doses comprises 600 mg, 1200 mg, or 1800 mg of isankizumab, and wherein when the method is compared to treatment with placebo in a population of human subjects suffering from moderately to severely active UC, a statistically significantly higher percentage of the human subjects treated with the method achieves remission 4 weeks, 8 weeks, or 12 weeks following the administration of the first induction dose.

Further benefits of the present disclosure will be apparent to one skilled in the art from reading this patent application. The embodiments of the disclosure described in the following paragraphs are intended to illustrate the invention and should not be deemed to narrow the scope of the invention.

Methods of treating inflammatory diseases including Crohn's Disease and ulcerative colitis using risankizumab have been described in U.S. Patent Application Publication No. 2018/0105588 A1 and U.S. Patent Application Publication No. 2017/0081402 A1, the administration, methods of manufacture and use of which are incorporated by reference in their entirety.

As previously noted, despite the availability of various Crohn's disease therapies, including biologic therapies such as anti-TNFs, many patients still do not respond adequately to these treatments, or gradually lose response over time. The methods of the present disclosure provide additional therapeutic options for Crohn's disease patients with inadequate response or intolerance to conventional therapies and biologic therapies.

Further, as previously noted, despite the availability of various ulcerative colitis therapies, including biologic therapies such as anti-TNFs, many patients still do not respond adequately to these treatments, or gradually lose response over time. The methods of the present disclosure provide additional therapeutic options for ulcerative colitis patients with inadequate response or intolerance to conventional therapies and biologic therapies.

Risankizumab

The CDRs of risankizumab used in the context of the present disclosure are shown in Tables 1 and 2. The variable regions of risankizumab used in the context of the present disclosure are shown in Table 3.

TABLE 1
Light Chain CDR Sequences

| L-CDR1 | L-CDR2 | L-CDR3 |
|---|---|---|
| KASRDVAIAVA | WASTRHT | HQYSSYPFT |
| (SEQ ID NO: 1) | (SEQ ID NO: 2) | (SEQ ID NO: 3) |

TABLE 2
Heavy Chain CDR Sequences

| H-CDR1 | H-CDR2 | H-CDR3 |
|---|---|---|
| GYTFTDQTIH | YIYPRDDSPKYNENFKG | PDRSGYAWFIY |
| (SEQ ID NO: 4) | (SEQ ID NO: 5) | (SEQ ID NO: 6) |

TABLE 3

| | |
|---|---|
| Humanized | DIQMTQSPSSLSASVGDRVTITCKASRDVAIAVAWYQQKP |
| VL | GKVPKLLIYWASTRHTGVPSRFSGSGSRTDFTLTISSLQP |
| Sequence | EDVADYFCHQYSSYPFTFGSGTKLEIK |
| | (SEQ ID NO: 7) |
| Humanized | QVQLVQSGAEVKKPGSSVKVSCKASGYTFTDQTIHWMRQA |
| VH | PGQGLEWIGYIYPRDDSPKYNENFKGKVTITADKSTSTAY |
| Sequence | MELSSLRSEDTAVYYCAIPDRSGYAWFIYWGQGTLVTVSS |
| | (SEQ ID NO: 8) |

Risankizumab comprises the heavy and light chain sequences shown in Table 4.

TABLE 4

| | |
|---|---|
| IgK | DIQMTQSPSSLSASVGDRVTITCKASRDVAIAVAWYQ |
| light | QKPGKVPKLLIYWASTRHTGVPSRFSGSGSRTDFTLT |
| Chain | ISSLQPEDVADYFCHQYSSYPFTFGSGTKLEIKRTVA |
| | APSVFIFPPSDEQLKSGTASVVCLLNNFYPREAKVQW |
| | KVDNALQSGNSQESVTEQDSKDSTYSLSSTLTLSKAD |
| | YEKHKVYACEVTHQGLSSPVTKSFNRGEC (SEQ ID |
| | NO: 9) |
| IgG1 KO | QVQLVQSGAEVKKPGSSVKVSCKASGYTFTDQTIHWM |
| Heavy | RQAPGQGLEWIGYIYPRDDSPKYNENFKGKVTITADK |
| Chain | STSTAYMELSSLRSEDTAVYYCAIPDRSGYAWFIYWG |
| | QGTLVTVSSASTKGPSVFPLAPSSKSTSGGTAALGCL |
| | VKDYFPEPVTVSWNSGALTSGVHTFPAVLQSSGLYSL |
| | SSVVTVPSSSLGTQTYICNVNHKPSNTKVDKRVEPKS |
| | CDKTHTCPPCPAPEAAGGPSVFLFPPKPKDTLMISRT |
| | PEVTCVVVDVSHEDPEVKFNWYVDGVEVHNAKTKPRE |
| | EQYNSTYRVVSVLTVLHQDWLNGKEYKCKVSNKALPA |
| | PIEKTISKAKGQPREPQVYTLPPSREEMTKNQVSLTC |
| | LVKGFYPSDIAVEWESNGQPENNYKTTPPVLDSDGSF |
| | FLYSKLTVDKSRWQQGNVFSCSVMHEALHNHYTQKSL |
| | SLSPG (SEQ ID NO: 10) |

Light chain and heavy chain variable region are underlined in Table 4 above.

Risankizumab is a fully humanized mAb of the IgG1 subclass directed towards IL-23p19. The antibody has been engineered to reduce Fcγ receptor and complement binding and potential charge heterogeneity. Risankizumab binds with high affinity to human IL-23 and inhibits IL-23 stimulated IL-17 production at inhibitory concentration (IC)₅₀ concentrations below 10 pM, as compared with 167 pM for ustekinumab in the same system. Risankizumab does not affect IL-12 at a maximum tested concentration (33 nM) and it does not inhibit IL-12 stimulated IFN-γ production.

### Definitions

Where a numeric range is recited herein, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated.

The singular forms “a,” “an” and “the” include plural referents unless the context clearly dictates otherwise.

The term “and/or” as used in a phrase such as “A and/or B” herein is intended to mean “A and B”, “A or B”, “A” or “B”.

The term “about” generally refers to a range of numbers that one of skill in the art would consider equivalent to the recited value (i.e., having the same function or result). In many instances, the term “about” may include numbers that are rounded to the nearest significant figure. In certain instances, the term “about” may be used to denote values falling within ±20% of the recited values, e.g. within ±15%, ±10%, ±7.5%, ±5%, ±4%, ±3%, ±2% or ±1% of the recited values.

The term “baseline” means the first measurement of the targeted variable just before the administration of the studied therapy.

Unless the context requires otherwise, the terms “comprise,” “comprises,” and “comprising” are used on the basis and clear understanding that they are to be interpreted inclusively, rather than exclusively, such that they indicate the inclusion of the recited feature but without excluding one or more other such features.

The term “patient”, “subject”, “individual” and the like refers to humans.

### Crohn's Disease

Induction Dosing

In one embodiment, the present disclosure relates to a method of treating Crohn's disease in a subject comprising administering to the subject at least one induction dose of risankizumab. In another embodiment, the present disclosure relates to a method of inducing remission of Crohn's disease in a subject comprising administering to the subject at least one induction dose of risankizumab. In another embodiment, the present disclosure relates to a method of inducing an endoscopic response in a subject comprising administering to the subject at least one induction dose of isankizumab.

The induction dose of risankizumab may comprise about 600 mg or about 1200 mg of risankizumab. In certain embodiments, the induction dose of risankizumab comprises about 1200 mg of isankizumab.

In one embodiment, the methods described in the present disclosure are used to treat or induce remission in a subject afflicted with moderately to severely active Crohn's disease. Moderately to severely active Crohn's disease may be defined as: (1) average daily stool frequency (SF) ≥4 and/or average daily abdominal pain (AP) score ≥2; plus (2) endoscopic evidence of mucosal inflammation as documented by the Simple Endoscopic Score for CD (SES-CD) ≥3. Accordingly, in one embodiment, the methods described in the present disclosure are used to treat a subject who has: (1) average daily stool frequency (SF) ≥4 and/or average daily abdominal pain (AP) score ≥2; plus (2) Simple Endoscopic Score for CD (SES-CD) ≥3. Alternatively, the subject has: (1) average daily stool frequency (SF) ≥4 and/or average daily abdominal pain (AP) score ≥2; plus (2) Simple Endoscopic Score for CD (SES-CD) ≥6 or ≥4 for isolated ileal disease; before the administration of the at least one induction dose of risankizumab. In another embodiment, the subject has Crohn's disease activity index (CDAI) score 220-450 at baseline (i.e., before the administration of the at least one induction dose of isankizumab).

In another embodiment, the subject treated with the methods described herein has intolerance or an inadequate response to one or more of anti-TNF or anti-integrin biologics for CD. The biologic therapies may include, but are not limited to, infliximab, adalimumab, certolizumab, vedolizumab, ustekinumab and/or natalizumab. Demonstration of intolerance may require no minimum dose or duration of use. Inadequate response may comprise signs and symptoms of persistently active disease despite a history of one or more of the following: at least one 6-week induction regimen of infliximab (≥5 mg/kg intravenous (IV) at Weeks 0, 2, and 6), at least one 4-week induction regimen of adalimumab (one 160 mg subcutaneous (SC) dose at Week 0, followed by one 80 mg SC dose at Week 2; or one 80 mg SC dose at Week 0, followed by one 40 mg SC dose at Week 2), at least one 4-week induction regimen of certolizumab pegol (400 mg SC at Weeks 0, 2, and 4), at least one 4-week induction regimen of golimumab (200 mg SC at Weeks 0 and 2), at least one 6-week induction regimen of vedolizumab (300 mg IV at Weeks 0, 2, and 6), at least one 12-week induction regimen of natalizumab (300 mg IV every 4 weeks), at least one 8-week induction regimen of ustekinumab [260 mg [symbol] 5 kg) or 390 mg (>55 to [symbol] 85 kg) or 520 mg (>85 kg) IV, followed by 90 mg SC at Week 8]; or may comprise recurrence of symptoms during scheduled maintenance dosing following prior clinical benefit of the above biologics.

In another embodiment, the subject has an inadequate response or intolerance to one or more conventional therapies selected from the group consisting of aminosalicylates (e.g., mesalamine, sulfasalazine, olsalazine, balsalazide), oral locally acting steroids (e.g., budesonide, beclomethasone), systemic corticosteroids (prednisone or equivalent), and immunomodulators (e.g., AZA, 6-MP, MTX).

In one embodiment, the method of treating Crohn's disease or inducing remission of Crohn's disease in a subject comprising administering to the subject 1 to 6 induction doses of risankizumab. Thus, the method may comprise administering to the subject 1, 2, 3, 4, 5, or 6 induction doses of risankizumab. In one embodiment, 3 induction doses are administered to the subject. In another embodiment, 6 induction doses are administered to the subject.

In one embodiment, at least one induction dose of risankizumab is administered at 4-week intervals. In one embodiment, all induction doses of risankizumab are administered at 4-week intervals.

For example, and without limitation, the method may comprise administering to the subject an induction dose comprising about 600 mg or about 1200 mg of risankizumab monthly for up to 6 months. In some embodiments, the method may comprise administering to the subject an induction dose comprising about 600 mg or about 1200 mg of risankizumab at week 0, 4, and 8; at week 0, 4, 8, and 12; at week 0, 4, 8, 12, and 16; or at week 0, 4, 8, 12, 16, and 20.

In one embodiment, at least one induction dose of risankizumab is administered via intravenous infusion (IV). In one embodiment, all induction doses of risankizumab are administered via intravenous infusion.

In one embodiment, the method of treating Crohn's disease or inducing remission of Crohn's disease in a subject further comprises measuring clinical, and/or endoscopic endpoints of the subject after the induction dose is administered. In one embodiment, the clinical, and/or endoscopic endpoints are measured 4 weeks after each induction dose is administered. In certain embodiments, the clinical and/or endoscopic endpoints are measured 4 weeks after the last (e.g., the third) induction dose is administered.

Clinical Endpoints

The clinical endpoints may comprise clinical remission (defined by average daily SF [symbol] 2.8 and not worse than Baseline AND average daily AP score [symbol] 1 and not worse than Baseline); enhanced clinical response (defined by [symbol] 60% decrease in average daily SF and/or [symbol] 35% decrease in average daily AP score and both not worse than Baseline, and/or clinical remission); and/or clinical response (defined by [symbol] 30% decrease in average daily SF and/or [symbol] 30% decrease in average daily AP score). Endoscopic endpoints may comprise endoscopic remission (defined by SES-CD [symbol] 4 and at least a 2 point reduction versus baseline and no subscore greater than 1 in any individual variable); endoscopic response (defined by decrease in SES-CD >50% from Baseline, or for subjects with isolated ileal disease and a Baseline SES-CD of 4, at least a 2 point reduction from Baseline); and/or ulcer-free endoscopy (defined by SES-CD ulcerated surface subscore of 0 in subjects with SES-CD ulcerated surface subscore [symbol] 1 at Baseline). Endpoints may also comprise CDAI clinical response (defined by reduction of CDAI [symbol] 100 points from baseline); CDAI clinical remission (defined by CDAI <150); SF remission (defined by average daily SF [symbol] 2.8 and not worse than baseline); and/or AP remission (defined by average daily AP score [symbol] 1 and not worse than baseline).

The subject may achieve one or more of the above clinical, histologic, and/or endoscopic endpoints after administration of the at least one induction dose of risankizumab. In one embodiment, the subject achieves clinical response (defined by [symbol] 30% decrease in average daily SF and/or [symbol] 30% decrease in average daily AP score) after administration of at least one induction dose of isankizumab. In one embodiment, the subject achieves one or more of the above clinical, histologic, and/or endoscopic endpoints after administration of 3 induction doses of about 600 or about 1200 mg of isankizumab. In certain embodiments, the subject achieves one or more of the above clinical, histologic, and/or endoscopic endpoints after administration of 3 induction doses of about 1200 mg of risankizumab.

In some embodiments, when a population of patients is treated, 30-60% of patients may achieve clinical remission (CDAI or SF/APS). In another embodiment, when a population of patients is treated, 30-60% (e.g., 30%, 35%, 40%, 45%, 50%, 55%, 60%) of patients achieve clinical as measured by CDAI or SF/APS. In certain embodiments, when a population of patients is treated with three induction doses of about 600 mg risankizumab, 40-50% (e.g., about 42%) of patients achieve clinical remission as measured by CDAI or SF/APS. In another embodiment, when a population of patients is treated with three induction doses of about 1200 mg of isankizumab, 40-50% (e.g., about 41.9%) of patients achieve clinical remission as measured by CDAI or SF/APS.

Maintenance Dosing

In one embodiment, the method of treating Crohn's disease or inducing remission of Crohn's disease in a subject further comprises (1) administering to the subject a first maintenance dose of risankizumab after the last induction dose is administered, and (2) administering at least one additional maintenance dose to the subject after the first maintenance dose is administered.

In one embodiment, the first maintenance dose is administered 2 to 8 weeks after the last induction dose is administered. Thus, the first maintenance dose may be administered 2, 3, 4, 5, 6, 7, or 8 weeks after the last induction dose is administered. In certain embodiments, the first maintenance dose may be administered 4 weeks after the last induction dose is administered.

In one embodiment, the first maintenance dose comprises about 180 mg or about 360 mg of risankizumab. In certain embodiments, the first maintenance dose may comprise about 360 mg of risankizumab. In further embodiments, the first maintenance dose may comprise about 180 mg of risankizumab.

In one embodiment, 4 to 12 weeks after the first maintenance dose is administered, at least one additional maintenance dose is administered to the subject. Thus, the at least one additional maintenance dose may be administered 4, 5, 6, 7, 8, 9, 10, 11, or 12 weeks after the first maintenance dose is administered. In certain embodiments, the at least one additional maintenance dose is administered 8 weeks after the first maintenance dose is administered.

At least one (e.g. 1, 2, 3, 4, 5, 6, 7, 8, 9, 10, 11, or 12) additional maintenance dose may be administered after the first maintenance dose. In one embodiment, 6 additional maintenance doses are administered after the first maintenance dose. In another embodiment, more than 6 additional maintenance doses are administered after the first maintenance dose. If more than one additional maintenance doses are administered, the additional maintenance doses may be administered at 8 weeks intervals.

In one embodiment, the additional maintenance dose comprises about 180 mg or about 360 mg of risankizumab. In certain embodiments, the additional maintenance dose may comprise about 360 mg of isankizumab. In further embodiments, the additional maintenance dose may comprise about 180 mg of risankizumab.

For example, and without limitation, the method may comprise administering to the subject an induction dose comprising about 600 mg or about 1200 mg of as described herein followed by a monthly or bi-monthly maintenance dose. In some such non-limiting examples, the method may comprise administering to the subject an induction dose comprising about 600 mg or about 1200 mg of risankizumab at week 0, 4, and 8; followed by a maintenance dose comprising about 180 mg or about 360 mg of risankizumab at week 12, and every 8 weeks thereafter.

In one embodiment, the first maintenance dose is administered by subcutaneous injection (SC). In another embodiment, at least one additional maintenance dose is administered by subcutaneous injection. In certain embodiments, all additional maintenance doses are administered by subcutaneous injection. In another embodiment, the first maintenance dose and all additional maintenance doses are administered by subcutaneous injection.

In one embodiment, the method further comprises measuring clinical, histologic, and/or endoscopic endpoints of the subject after the maintenance dose is administered. In one embodiment, the clinical, histologic, and/or endoscopic endpoints are measured 4 weeks after the maintenance dose is administered. In certain embodiments, the clinical, histologic, and/or endoscopic endpoints are measured 4 weeks after the last maintenance dose is administered. In certain embodiments, 6 additional maintenance doses are administered after the first maintenance dose, and the clinical, histologic, and/or endoscopic endpoints are measured 4 weeks after the administration of the sixth additional maintenance dose.

The subject treated with the methods of the present disclosure may maintain one or more of the clinical, histologic, and/or endoscopic endpoints described herein after the administration of the maintenance doses of isankizumab. In one embodiment, the subject maintains the clinical response after the administration of the maintenance doses of isankizumab. In certain embodiments, the subject may maintain one or more of the clinical, histologic, and/or endoscopic endpoints described herein after the administration of the first maintenance dose and 6 additional maintenance doses of risankizumab.

Ulcerative Colitis

Induction Dosing

In one embodiment, the present disclosure relates to a method of treating ulcerative colitis in a subject comprising administering to the subject at least one induction dose of risankizumab. In another embodiment, the present disclosure relates to a method of inducing remission of ulcerative colitis in a subject comprising administering to the subject at least one induction dose of risankizumab.

The induction dose of risankizumab may comprise about 600 mg, about 1200 mg or about 1800 mg of risankizumab. In one embodiment, the induction dose of risankizumab comprises about 1200 mg of risankizumab. In another embodiment, the induction dose of risankizumab comprises about 1800 mg of risankizumab.

In one embodiment, the methods described in the present disclosure are used to treat or induce remission in a subject afflicted with moderately to severely active ulcerative colitis. Moderately to severely active ulcerative colitis may be defined as Adapted Mayo score of 5 to 9 points (using the Mayo scoring system, excluding Physician's Global Assessment) with an endoscopic subscore of 2 to 3 (e.g., 2 or 3) on screening endoscopy. Accordingly, in one embodiment, the methods described in the present disclosure are used to treat a subject who has an Adapted Mayo score of 5 to 9 points and an endoscopic subscore of 2 to 3 (e.g., 2 or 3).

In another embodiment, the subject treated with the methods described herein has intolerance or an inadequate response to one or more of biologic therapies for ulcerative colitis. The biologic therapies may include, but are not limited to, infliximab, adalimumab, golimumab, tofacitinib, and/or vedolizumab. Demonstration of intolerance may require no minimum dose or duration of use. Inadequate response may comprise signs and symptoms of persistently active disease despite a history of one or more of the following: at least one 6-week induction regimen of infliximab (≥5 mg/kg intravenous [IV] at Weeks 0, 2, and 6), at least one 4-week induction regimen of adalimumab (one 160 mg subcutaneous [SC] dose at Week 0, followed by one 80 mg SC dose at Week 2, or one 80 mg SC dose at Week 0, followed by one 40 mg SC dose at Week 2), at least one 4-week induction regimen of golimumab (200 mg SC at Weeks 0 and 2), at least one 6-week induction regimen of vedolizumab (300 mg IV at Weeks 0, 2, and 6), or recurrence of symptoms during scheduled maintenance dosing following prior clinical benefit of the above biologics.

In another embodiment, the subject treated with the methods described herein has an inadequate response or intolerance to conventional therapy. Conventional therapy is defined as one or more of the following: aminosalicylates, oral locally acting steroids (e.g., budesonide, beclomethosone), systemic corticosteroids (prednisone or equivalent), or immunomodulators. This population may also include subjects who have received biologic therapy or tofacitinib in the past but stopped therapy based on reasons other than inadequate response or intolerance (e.g., change in reimbursement coverage, well-controlled disease).

In one embodiment, the method of treating ulcerative colitis or inducing remission of ulcerative colitis in a subject comprising administering to the subject 1 to 6 induction doses of risankizumab. Thus, the method may comprise administering to the subject 1, 2, 3, 4, 5, or 6 induction doses of risankizumab. In one embodiment, 3 induction doses are administered to the subject. In another embodiment, 6 induction doses are administered to the subject.

In one embodiment, at least one induction dose of risankizumab is administered at 4-week intervals. In one embodiment, all induction doses of risankizumab are administered at 4-week intervals.

In one embodiment, at least one induction dose of risankizumab is administered via intravenous infusion (IV). In one embodiment, all induction doses of risankizumab are administered via intravenous infusion.

In one embodiment, the method of treating ulcerative colitis or inducing remission of ulcerative colitis in a subject further comprises measuring clinical, endoscopic, and/or histologic endpoints of the subject after the induction dose is administered. In one embodiment, the clinical, endoscopic, and/or histologic endpoints are measured 4 weeks after each induction dose is administered. In certain embodiments, the clinical, endoscopic, and/or histologic endpoints are measured 4 weeks after the third induction dose is administered.

Clinical Endpoints

The clinical endpoints may comprise clinical remission per Adapted Mayo (defined by a stool frequency subscore (SFS) ≤1 and not greater than baseline, a rectal bleeding subscore (RBS)=0, and an endoscopic subscore ≤1); clinical response per Adapted Mayo (defined by a decrease from baseline in the Adapted Mayo score ≥2 points and ≥30% from baseline, plus a decrease in a rectal bleeding subscore (RBS) ≥1 or an absolute RBS ≤1); clinical response per partial Adapted Mayo (defined by a decrease from baseline in the Adapted Mayo score ≥1 points and ≥30% from baseline, plus a decrease in a RBS ≥1 or an absolute RBS ≤1); or clinical remission per Full Mayo (defined by Full Mayo score ≤2 with no subscore >1). Endoscopic endpoints may comprise endoscopic improvement (defined by an endoscopy subscore of 0 or 1) or endoscopic remission (defined by an endoscopy subscore of 0). Histologic endpoints may comprise histologic remission (defined by a Geboes score <2). Endpoints may also comprise mucosal healing, which is defined by the endoscopic and histologic remission.

The subject may achieve one or more of the above clinical, endoscopic, and/or histologic endpoints after administration of the at least one induction dose of risankizumab. In one embodiment, the subject achieves clinical remission per Adapted Mayo after administration of at least one induction dose of isankizumab. In another embodiment, the subject achieves clinical response per Adapted Mayo after administration of at least one induction dose of isankizumab. In one embodiment, the subject achieves one or more of the above clinical, endoscopic, and/or histologic endpoints after administration of 3 induction doses of about 600 mg, about 1200 mg or about 1800 mg of risankizumab.

In one embodiment, when a population of patients is treated, 2.5%-20% (e.g., 2.5%-15%, 5%-20%, 5%-15%, or 8%-12%) of patients may achieve clinical remission per Adapted Mayo Score. In certain embodiment, when a population of patients is treated with three induction doses of about 1200 mg of risankizumab, 5%-20% (e.g., about 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, or 20%) of patients achieve clinical remission per Adapted Mayo Score. In certain embodiment, when a population of patients is treated with three induction doses of about 1800 mg of risankizumab, 2.5%-15% (e.g., about 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15%) of patients achieve clinical remission per Adapted Mayo Score.

In another embodiment, when a population of patients is treated, 14%-16% (e.g., about 14%, about 14.5%, about 14.8%, about 15%, about 15.5%, or about 16%) of the population achieve endoscopic improvement. In another embodiment, when a population of patients is treated, 5%-9% (e.g., about 5%, about 5.5%, about 5.7%, about 6%, about 6.5%, about 7%, about 7.5%, about 8%, about 8.5%, or about 9%) of the population achieve clinical remission per Full Mayo score. In another embodiment, when a population of patients is treated, 47%-54% (e.g., about 47%, about 47.5%, about 48%, about 48.5%, about 49%, about 49.5%, about 50%, about 50.5%, about 51%, about 51.5%, about 52%, about 52.5%, about 53%, about 53.4%, about 53.5%, or about 54%) of the population achieve Clinical Response per Adapted Mayo Score. In another embodiment, when a population of patients is treated, 37%-46% (e.g., about 37%, about 37.5%, about 37.9%, about 38%, about 38.5%, about 39%, about 39.5%, about 40%, about 40.5%, about 41%, about 41.5%, about 42%, about 42.5%, about 43%, about 43.5%, about 44%, about 44.5%, about 45%, about 45.5%, about 45.9%, or about 46%) of the population achieve Clinical Response per Partial Adapted Mayo Score. In another embodiment, when a population of patients is treated, 4%-7% (about 4%, about 4.5%, about 4.9%, about 5%, about 5.5%, about 6%, about 6.5%, about 6.9%, or about 7%) of the population achieve Endoscopic Remission. In another embodiment, when a population of patients is treated, 4%-5% (e.g., about 4%, about 4.3%, about 4.4%, about 4.5%, or about 5%) of the population achieve change from Baseline in Inflammatory Bowel Disease Questionnaire (IBDQ).

Maintenance Dosing

In one embodiment, the method of treating ulcerative colitis or inducing remission of ulcerative colitis in a subject further comprises (1) administering to the subject a first maintenance dose of risankizumab after the last induction dose is administered, and (2) administering at least one additional maintenance dose to the subject after the first maintenance dose is administered.

In one embodiment, the first maintenance dose is administered 2 to 8 weeks after the last induction dose is administered. Thus, the first maintenance dose may be administered 2, 3, 4, 5, 6, 7, or 8 weeks after the last induction dose is administered. In certain embodiments, the first maintenance dose may be administered 4 weeks after the last induction dose is administered.

In one embodiment, the first maintenance dose comprises about 180 mg or about 360 mg of risankizumab. In certain embodiments, the first maintenance dose may comprise about 180 mg of isankizumab. In certain embodiments, the first maintenance dose may comprise about 360 mg of risankizumab.

In one embodiment, 4 to 12 weeks after the first maintenance dose is administered, at least one additional maintenance dose is administered to the subject. Thus, the at least one additional maintenance dose may be administered 4, 5, 6, 7, 8, 9, 10, 11, or 12 weeks after the first maintenance dose is administered. In certain embodiments, the at least one additional maintenance dose is administered 8 weeks after the first maintenance dose is administered.

At least one (e.g. 1, 2, 3, 4, 5, 6, 7, 8, 9, 10, 11, or 12) additional maintenance dose may be administered after the first maintenance dose. In one embodiment, 6 additional maintenance doses are administered after the first maintenance dose. In another embodiment, more than 6 additional maintenance doses are administered after the first maintenance dose. If more than one additional maintenance doses are administered, the additional maintenance doses may be administered at 8-week intervals.

In one embodiment, the additional maintenance dose comprises about 180 mg or about 360 mg of risankizumab. In certain embodiments, the additional maintenance dose may comprise about 360 mg of risankizumab.

In one embodiment, the first maintenance dose is administered by subcutaneous injection (SC). In another embodiment, at least one additional maintenance dose is administered by subcutaneous injection. In certain embodiments, all additional maintenance doses are administered by subcutaneous injection. In another embodiment, the first maintenance dose and all additional maintenance doses are administered by subcutaneous injection.

In one embodiment, the method further comprises measuring clinical, endoscopic, and/or histologic endpoints of the subject after the maintenance dose is administered. In one embodiment, the clinical, endoscopic, and/or histologic endpoints are measured 4 weeks after the maintenance dose is administered. In certain embodiments, the clinical, endoscopic, and/or histologic endpoints are measured 4 weeks after the last maintenance dose is administered. In certain embodiments, 6 or more additional maintenance doses are administered after the first maintenance dose, and the clinical, endoscopic, and/or histologic endpoints are measured 4 weeks after the administration of the sixth additional maintenance dose.

The subject treated with the methods of the present disclosure may maintain one or more of the clinical, endoscopic, and/or histologic endpoints described herein after the administration of the maintenance doses of isankizumab. In one embodiment, the subject maintains the clinical remission per Adapted Mayo after the administration of the maintenance doses of risankizumab. In another embodiment, the subject maintains the clinical response per Adapted Mayo after the administration of the maintenance doses of risankizumab. In certain embodiments, the subject may maintain one or more of the clinical, endoscopic, and/or histologic endpoints described herein after the administration of the first maintenance dose and 6 additional maintenance doses of risankizumab.

### Example 1: Methods of the Phase 2 Study M15-989

Study Design and Patients

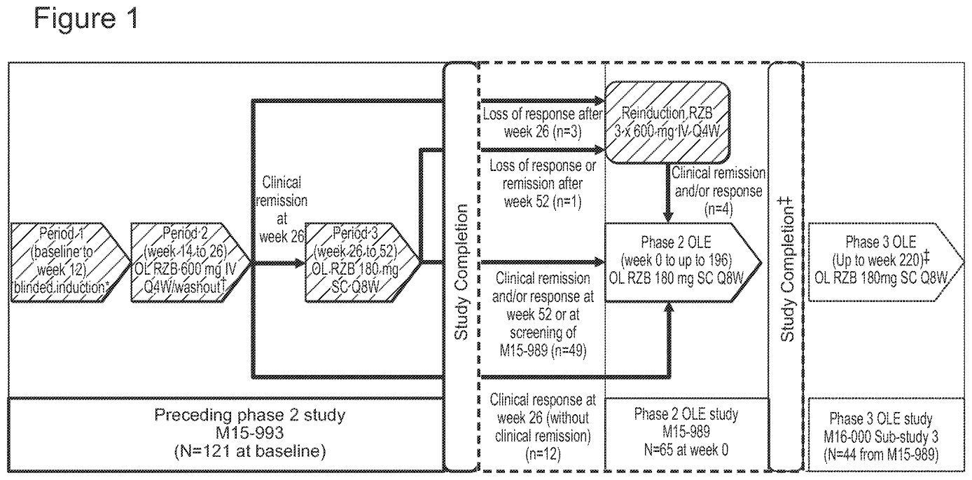

Patients who successfully completed the phase 2 M15-993 study (NCT02031276) were eligible to enroll in this single-group OLE (M15-989; NCT02513459; US Clinical Trials Registry). Patients were included from 38 referral sites across Europe, Asia, and North America. All patients received maintenance therapy with open-label risankizumab 180 mg SC every 8 weeks (Q8W) up to 196 weeks (FIG. 1). Successful completion of M15-993 was defined as achievement of clinical response (decrease in Crohn's Disease Activity Index [CDAI] from baseline with [symbol] 100 points) without clinical remission (CDAI <150) at week 26 or achievement of clinical response and/or remission at week 52 (Feagan et al. (2018) Lancet Gastroenterol Hepatol. 3:671-80). Patients who had lost clinical remission at the end of the trial visit for the M15-993 study, or at screening for the M15-989 OLE, received open-label reinduction of risankizumab with three infusions of 600 mg IV Q4W, after which eligibility was reassessed. If clinical response or remission was achieved, subjects were switched to maintenance treatment of risankizumab 180 mg SC Q8W from visit 5 for the remainder of the OLE. Patients who completed the M15-989 study could elect to enroll in the M16-000 sub-study 3, an ongoing phase 3 OLE (NCT03105102), in which they continue to receive open-label risankizumab 180 mg SC Q8W and are eligible to receive risankizumab rescue therapy. The M15-989 study was terminated by the sponsor to provide the option for patients to roll over for continuous open-label treatment within the ongoing phase 3 OLE.

Inclusion and exclusion criteria in the parent M15-993 study have been previously described (Feagan et al. (2017) Lancet. 389:1699-709). In brief, patients were adults (aged 18-75 years) with moderate-to-severe CD, defined as CDAI 220-450 and Crohn's Disease Endoscopic Index of Severity (CDEIS) ≥7 (or ≥4 for patients with isolated ileitis) with mucosal ulcers in the ileum and/or colon. Patients could have been biologic-naïve or previously treated with one or more TNF antagonists or vedolizumab, while patients who had received ustekinumab were ineligible for enrollment.

During the OLE, patients were permitted to continue oral corticosteroids (prednisone equivalent dose of ≤20 mg/day), oral 5-aminosalicylates, and immunosuppressive agents (azathioprine, 6-mercaptopurine, or methotrexate) if they had been on a stable dose for at least 4 weeks prior to visit 2, and were required to continue the same dose throughout. Initiation of systemic antihistamines and IV steroids was permitted in the case of infusion reactions during or after risankizumab infusion in accordance with the severity of the reaction and the local standard of care.

The M15-989 OLE was conducted in accordance with the International Conference on Harmonization guidelines, applicable regulations, and the Declaration of Helsinki. Study-related documents were approved by institutional ethics committees and review boards. All patients provided written, informed consent. An independent data monitoring committee was utilised to ensure that the welfare of patients participating in this study was maintained.

Outcomes

The primary objective was to assess long-term safety in patients with CD receiving maintenance risankizumab 180 mg SC Q8W. Adverse events (AEs), vital signs, and laboratory assessments were collected throughout the study and up to 20 weeks after the last dose of risankizumab for patients who prematurely discontinued the study. AEs were coded using the Medical Dictionary for Drug Regulatory Activities version 211, and severity was graded based on the Rheumatology Common Toxicity Criteria version 4-0. Major cardiac, cerebrovascular, and thrombotic events were adjudicated by an independent cardiovascular adjudication committee.

Treatment-emergent AEs (TEAEs) were defined as events occurring or worsening either on or after the first dose of risankizumab in the M15-989 study; within 20 weeks after the last dose of risankizumab for patients who prematurely terminated the study drug in the M15-989 study; and before the first dose of risankizumab in the phase 3 OLE. Duration of risankizumab exposure was defined as the date of last risankizumab dose minus the date of first risankizumab dose in the M15-989 OLE plus 8 weeks. AEs are presented as the number of patients (%) experiencing any event, the absolute number of events, and events per 100 patient-years (E/100 PY).

Secondary objectives included the assessment of pharmacokinetics, immunogenicity, efficacy, and HRQoL. Plasma samples were collected at scheduled visits, and were analysed at a later date using an enzyme-linked immunosorbent assay to measure risankizumab plasma concentration and an electrochemiluminescence assay with a three-tiered approach for the detection of ADAs (Feagan et al. (2017) Lancet. 389:1699-709). Due to sparse sampling, only trough plasma concentrations at the time relative to the first SC dose were summarised. Key efficacy outcomes included clinical remission (CDAI <150) and endoscopic remission (CDEIS ≥4, or ≥2 for patients with isolated ileitis); HRQoL outcomes included Inflammatory Bowel Disease Questionnaire (IBDQ) response (increase from baseline in IBDQ total score ≤16) and remission (IBDQ total score ≤170); biomarker outcomes included median change from baseline in serum albumin, high-sensitivity C-reactive protein (hs-CRP), and faecal calprotectin. CDAI scores and hs-CRP levels were assessed at every visit; IBDQ scores and serum albumin concentrations were assessed every 24 weeks; faecal calprotectin was assessed at baseline, week 24, and every 32 weeks thereafter; and ileocolonoscopies were performed yearly. Central scoring of endoscopy results was performed by independent, blinded reviewers for all endoscopic assessments.

Statistical Analysis

Safety data were reported for patients who received at least one dose of risankizumab (safety analysis set). The pharmacokinetics and immunogenicity analysis set included all patients who had pharmacokinetics and ADA data. Efficacy and HRQoL data were reported for the intent-to-treat population with non-responder imputation (NRI) of missing data up to week 128, as from that point onwards patients could roll over to the phase 3 OLE. Only observed cases were reported beyond week 128. Note that the final time point reported may vary for different efficacy outcomes according to the frequency of assessments described in the previous section, and that patients completed M15-989 at different time points to roll over into the phase 3 OLE. No sample size calculation or statistical comparisons were conducted; all analyses were summarised descriptively. Week 0 denotes the first dose of study drug in the M15-989 OLE. However, for baseline characteristics and changes from baseline, baseline was defined as being prior to the first dose of study drug in the parent M15-993 study (Feagan et al. (2017) Lancet. 389:1699-709).

### Example 2: Results of the Phase 2 Study M15-989

### Summary

Background: Risankizumab, an interleukin-23 antibody, demonstrated efficacy and acceptable safety in a phase 2 study of patients with moderate-to-severe refractory Crohn's disease (CD). This open-label extension (OLE) aimed to investigate the long-term safety, pharmacokinetics, immunogenicity, and efficacy of risankizumab in patients who responded to risankizumab in the parent phase 2 study.

Methods: Patients who had clinical response (decrease in Crohn's Disease Activity Index [CDAI] from baseline [symbol] 100) without clinical remission (CDAI <150) at week 26, or clinical response and/or remission at week 52 in the parent phase 2 study rolled over to the OLE and received open-label subcutaneous risankizumab 180 mg every 8 weeks (Q8W). The primary objective of the OLE was to assess long-term safety of risankizumab, and secondary objectives included assessment of pharmacokinetics, immunogenicity, and efficacy.

Findings: Sixty-five patients were enrolled, including four patients who had lost response in the parent study and were first reinduced with three infusions of risankizumab 600 mg every 4 weeks. Patients received risankizumab for a median of 33 months, with a cumulative total of 167.0 patient-years (PY). The rate of serious adverse events was 24.6 E/100 PY; the majority were gastrointestinal in nature. Rates of serious infections, opportunistic infections, and fungal infections were 4.2, 1.8, and 6.6 E/100 PY, respectively. No deaths, malignancies, adjudicated cardiovascular events, or cases of latent/active tuberculosis or herpes zoster were reported. Risankizumab steady-state plasma exposures were reached by week 24 and were maintained throughout the study. Treatment-emergent anti-drug antibodies developed in eight patients (12.3%), but none were neutralising. Efficacy outcomes were maintained during the OLE, including the proportions of patients (observed analysis) with clinical remission (>71%) and endoscopic remission (>42%).

Interpretation: Long-term maintenance treatment with subcutaneous risankizumab 180 mg Q8W was well tolerated by patients with CD, with no specific safety signals.

Results

Patient Disposition

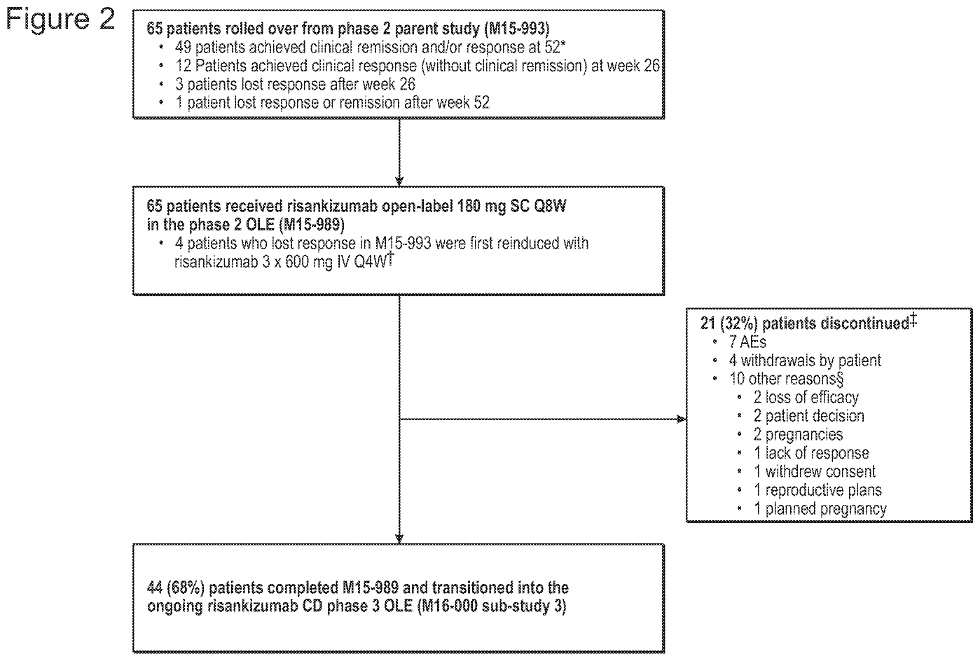

Of the 121 patients randomised in the M15-993 study (Feagan et al. (2018) Lancet Gastroenterol. Hepatol. 3:671-80), 65 rolled over to this OLE (all risankizumab group) from September 2015 onwards. This included four patients who had lost response at the end of the M15-993 study and were reinduced with risankizumab 600 mg IV Q4W at the beginning of the OLE (FIG. 1 and FIG. 2). Forty-four patients (68%) completed the M15-989 study and transitioned into the phase 3 OLE, while 21 (32%) patients prematurely discontinued the study. Seven patients discontinued the study due to AEs and seven patients withdrew consent; all reasons for discontinuation are detailed in FIG. 2. The M15-989 study concluded in June 2019; it was terminated by the sponsor to provide the option for patients to roll over to continuous open-label treatment within the ongoing phase 3 OLE.

Baseline characteristics and disease activity (ie, at entry into the M15-993 study) of the OLE population are presented in Table 5. Median CD duration was 10.0 years (range: 2.0-38.0), and 92.3% patients had been previously treated with at least one TNF antagonist.

TABLE 5
Baseline characteristics* (intent-to-treat analysis set)

| | All risankizumab | |
|---|---|---|
| | (n = 65) | |
| Age, years | 34.0 | (19-67) |
| Female | 36 | (55.4) |
| Race, white | 55 | (84.6) |
| Weight, kg | 66.0 | (41.0-123.9) |
| Disease duration, years | 10.0 | (2.0-38.0) |
| CD location | | |
| Ileocolonic | 37 | (56.9) |
| Colonic only | 20 | (30.8) |
| Ileum only | 8 | (12.3) |
| CDAI | 298.0 | (109.0-477.6) |
| CDEIS | 11.9 | (5.2-25.0) |
| IBDQ | 114.0 | (52.0-182.0) |
| hs-CRP, mg/L | 10.2 | (0.2-109.0) |
| Faecal calprotectin, mg/kg | 1364.0 | (40.0-25 252.0) |
| Concomitant treatment^{†} | | |
| Corticosteroid only | 13 | (20.0) |
| IMM only | 21 | (32.3) |
| Corticosteroid and IMM | 9 | (13.8) |
| Prior TNF antagonist use | 60 | (92.3) |
| Prior use of one TNF antagonist | 20 | (30.8) |
| Prior use of two TNF antagonists | 32 | (49.2) |
| Prior use of three TNF antagonists | 8 | (12.3) |

Data are n (%) or median (range).
*Baseline is defined as prior to first dose of study drug in the parent M15-993 study.
^{†}Patients were permitted to continue oral corticosteroids (prednisone equivalent dose of ≤20 mg/day) and immunomodulators (azathioprine, 6-mercaptopurine, or methotrexate) if they had been on a stable dose for at least 4 weeks prior to visit 2, and were required to continue the same dose throughout the M15-989 OLE.
CD = Crohn's disease; CDAI = Crohn's Disease Activity Index; CDEIS = Crohn's Disease Endoscopic Index of Severity; hs-CRP = high-sensitivity C-reactive protein; IBDQ = Inflammatory Bowel Disease Questionnaire; IMM = immunomodulatory; TNF = tumour necrosis factor.

Safety

In the all risankizumab group (n=65), the treatment duration of risankizumab ranged from 114 to 1317 days, with a median of 1014 days (33 months) and a total of 167.0 PY. Forty patients (62%) received risankizumab for >924 days. All patients who required IV reinduction (n=4) received the full reinduction with three infusions of 600 mg IV risankizumab Q4W (median duration: 84 days) and achieved clinical response by week 8; then continued risankizumab 180 mg SC Q8W from week 12 onwards.

Most patients experienced at least one TEAE (Table 6). The most common TEAEs were nasopharyngitis, gastroenteritis, and fatigue (Table 7). Serious AEs (SAEs) were reported in 23 patients (35.4%; 24.6 E/100 PY). SAEs observed in more than one patient were worsening CD, intestinal stenosis, viral gastroenteritis, peritonitis, and post-procedural complications (n=2 each). Six patients (9.2%; 4.2 E/100 PY) discontinued the study drug due to the following TEAEs: worsening CD (n=5), including one patient with colon injury (intraluminal laceration of the colon with no perforation during an endoscopy), and ileal stenosis (n=1). No deaths were reported.

TABLE 6
Overview of TEAEs (safety analysis set)

| | All risankizumab | | |
|---|---|---|---|
| | (n = 65; PY = 167.0) | | |
| | Number of patients (%) | | E/100 PY |
| Any AE | 60 | (92.3) | 408.4 |
| AE possibly related to study drug* | 26 | (40.0) | 49.7 |
| Serious AE | 23 | (35.4) | 24.6 |
| Severe AE^{†} | 14 | (21.5) | 15.0 |
| AE leading to discontinuation of | 6 | (9.2) | 4.2 |
| study drug | | | |
| All deaths | 0 | | 0 |
| Infection | 48 | (73.8) | 112.0 |
| Serious infection | 6 | (9.2) | 4.2 |
| Opportunistic infection | 3 | (4.6) | 1.8 |
| Fungal infection | 7 | (10.8) | 6.6 |
| Tuberculosis | 0 | | 0 |
| Herpes zoster | 0 | | 0 |
| Malignancies excluding NMSC | 0 | | 0 |
| Hypersensitivity | 16 | (24.6) | 10.2 |
| Anaphylactic reaction | 1^{‡} | (1.5) | 0.6 |
| Hepatic event | 6 | (9.2) | 10.2 |

| | | |
|---|---|---|
| Adjudicated MACE | 0 | 0 |

*As assessed by the investigator.
^{†}AEs with NCI toxicity ≥ Grade 3 or unknown severity.
^{‡}Serious anaphylactic reaction to IV iron carboxymaltose administration.
AE = adverse event; E/100 PY = events per 100 patient-years; IV = intravenous; MACE = major adverse cardiovascular event; NCI = National Cancer Institute; NMSC = non-melanoma skin cancer; PY = patient-years; TEAE = treatment-emergent adverse event.

TABLE 7
AEs reported in >10% of patients by Preferred Term
in order of decreasing frequency (safety analysis set)

| | All risankizumab | | |
|---|---|---|---|
| | (n = 65; PY = 167.0) | | |
| MedDRA21-1 Preferred Term | Number of patients (%) | | E/100 PY |
| Nasopharyngitis | 20 | (30.8) | 24.0 |
| Gastroenteritis | 15 | (23.1) | 11.4 |
| Fatigue | 13 | (20.0) | 8.4 |
| Abdominal pain | 12 | (18.5) | 7.8 |
| Crohn's disease | 12 | (18.5) | 9.0 |
| Arthralgia | 11 | (16.9) | 8.4 |
| Nausea | 10 | (15.4) | 6.0 |
| Headache | 9 | (13.8) | 9.0 |
| Urinary tract infection | 9 | (13.8) | 6.0 |
| Back pain | 8 | (12.3) | 6.0 |
| Diarrhoea | 8 | (12.3) | 5.4 |
| Influenza | 8 | (12.3) | 6.6 |
| Upper respiratory tract infection | 7 | (10.8) | 5.4 |

AE = adverse event; E/100 PY = eventsper 100 patient-years; MedDRA = Medical Dictionary for Regulatory Activities; PY = patient-years.

Infections were reported in 48 patients (73.8%; 112.0 E/100 PY). Serious infections were reported in six patients (9.2%; 4.2 E/100 PY); of these, pelvic peritonitis secondary to anal dilatation and viral gastroenteritis (reported in the same patient), peritonitis following ileocaecal resection surgery (one patient), and Campylobacter infection (one patient) were considered severe. The remaining serious infections were mild (subcutaneous abscess) or moderate (perianal abscess and viral gastroenteritis). None of the serious infections led to discontinuation of the study drug. Opportunistic infections were reported in three patients (4.6%; 1.8 E/100 PY) and included fungal oesophagitis and oral fungal infection; all were resolved during the study period. Fungal infections were reported in seven patients (10.8%; 6.6 E/100 PY). Oral fungal infection and vulvovaginal mycotic infection were most frequently reported (n=2 each). The remaining fungal infections were single events of Blastocystis infection, fungal infection of the hands and feet, fungal oesophagitis, fungal skin infection, tinea capitis, and vulvovaginal candidiasis. None of these were serious (all Grade 1 or 2), and none led to treatment discontinuation. No events of tuberculosis or herpes zoster infections were reported.

Hypersensitivity reactions were reported in 16 patients (24.6%; 10.2 E/100 PY). The most frequently reported hypersensitivity reactions were rash (n=5) and eczema (n=3). Five events were considered to be related to the study drug, and no events led to discontinuation of risankizumab treatment. All hypersensitivity reactions were assessed as Grade 1 or 2, except for one SAE, which was an anaphylactic reaction due to iron carboxymaltose (Ferinject®) administration.

Hepatic events were reported in six patients (9.2%; 10.2 E/100 PY); 15 events involved increased liver enzymes. A single event of hepatic steatosis was observed in a patient with a body mass index >30 kg/m². All hepatic events were assessed as Grade 1, none were serious, and none led to discontinuation of risankizumab. Potentially clinically important increases in liver enzymes were rare (Table 8), and increases in alanine aminotransferase and aspartate aminotransferase normalised during the OLE. No patients met the criteria for Hy's Law.

TABLE 8
Potentially clinically important laboratory values experienced
by ≥1 patient in M15-989 (safety analysis set)

| | All risankizumab | |
|---|---|---|
| | (n = 65) | |
| Lymphocytes, 10⁹/L | | |
| <0.5 | 3 | (4.6) |
| Creatine kinase, U/L | | |
| >5.0 × ULN | 6 | (9.2) |
| Potassium, mmol/L | | |
| >6.0 | 1 | (1.5) |
| <3.0 | 2 | (3.1) |
| Triglycerides, mmol/L | | |
| >5.7 | 5 | (7.7) |
| Gamma glutamyl transferase, | | |
| U/L | | |
| >5.0 × ULN | 1 | (1.5) |
| Alanine aminotransferase, U/L | | |
| ≥3.0 × ULN | 2 | (3.1) |
| Aspartate aminotransferase, U/L | | |
| ≥3.0 × ULN | 1 | (1.5) |
| Alkaline phosphatase, U/L | | |
| ≥1.5 × ULN | 2 | (3.1) |
| Bilirubin, μmol/L | | |
| ≥3.0 × ULN | 2 | (3.1) |

Data is n (%).
Potentially clinically important laboratory values were defined as those with Common Toxicity Criteria ≥Grade 3 and a higher grade than baseline.
ULN = upper limit of normal.

No malignancies or adjudicated major adverse cardiovascular events were reported during the OLE.

In general, changes in laboratory values were not considered clinically meaningful (Table 8). There were few individual cases of ≥Grade 3 laboratory values and most were transient. Three patients had ≥Grade 3 decreases in lymphocytes, of which two had ≥Grade 3 decreases only once during the study, and all decreases resolved without discontinuation. Six patients had ≥Grade 3 increases in creatine kinase, five of which experienced increases only once during the study, and three of which experienced increases during the follow-up period. All increases resolved without discontinuation. Most of these events were assessed by the investigator as temporary and not clinically meaningful. Five patients experienced ≥Grade 3 increases in triglycerides, one of which had increased triglycerides prior to this study. No persistent trend was observed. All increases in triglycerides resolved, and no patients discontinued due to increases in triglycerides.

Three pregnancies were reported during this OLE: two pregnancies occurred during maternal treatment with risankizumab 180 mg SC Q8W, and the third was the pregnancy of a patient's partner. Maternal use of risankizumab was discontinued after pregnancy was confirmed. One of the patients with maternal use of risankizumab pre-conception and during the first trimester (having received risankizumab treatment for 575 days in total) surgically terminated her pregnancy because of foetal defects (foetal cystic hygroma and hydrops fetalis) 78 days after risankizumab was discontinued; this was considered by the investigator as having a reasonable possibility of being related to risankizumab. This patient subsequently had a second pregnancy outside the OLE reporting period (after discontinuation of risankizumab for >20 weeks and therefore not included as one of the three pregnancies that occurred during the OLE), which resulted in a miscarriage. The other two pregnancies concluded with live births without any reported complications or abnormalities.

Pharmacokinetics and Immunogenicity

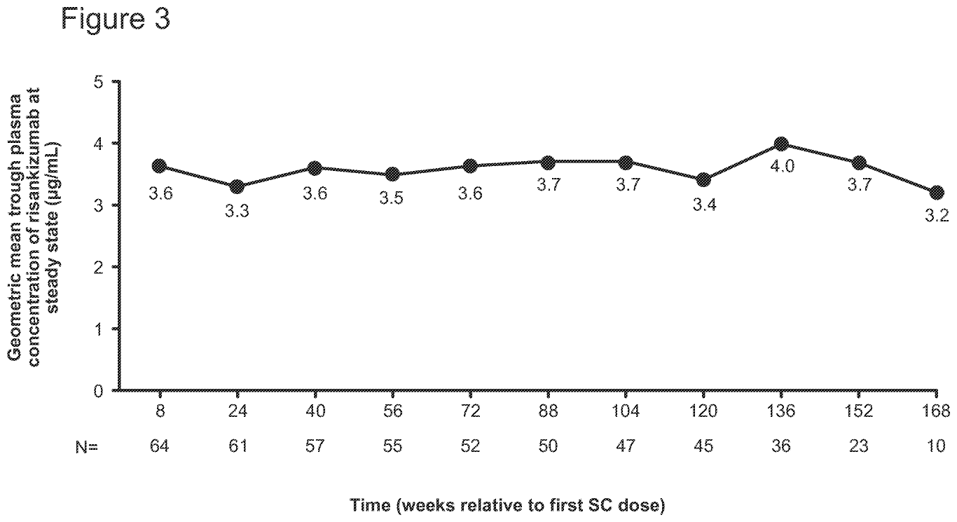

Steady-state plasma exposures of risankizumab were reached by week 24 and were maintained throughout the study, with geometric mean trough concentrations ranging from 3.2 to 4.0 μg/mL (FIG. 3).

At week 0 (prior to first risankizumab dose in M15-989), pre-existing ADAs were detected in 2165 patients (3%) who received at least one dose of risankizumab in the study, possibly due to prior exposure to risankizumab in the parent phase 2 study M15-993. Treatment-emergent ADAs developed in 8/65 patients (12%) during this OLE, none of whom developed neutralising antibodies. Four patients with treatment-emergent ADAs discontinued from the study for the following reasons: AE, pregnancy, withdrawal of consent, and lack of efficacy, respectively. ADA titres ranged from 1 to 64 with no apparent impact on risankizumab plasma exposure in all but one patient. Further pharmacokinetic and immunogenicity data is collected and their correlations with safety and efficacy outcomes are analysed in phase 3 studies.

Efficacy, HRQoL, and Biomarkers

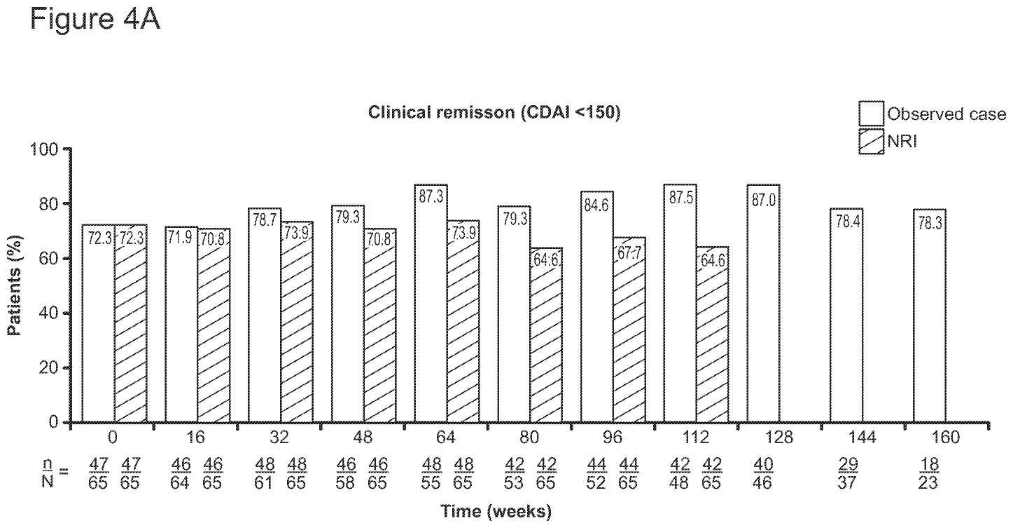

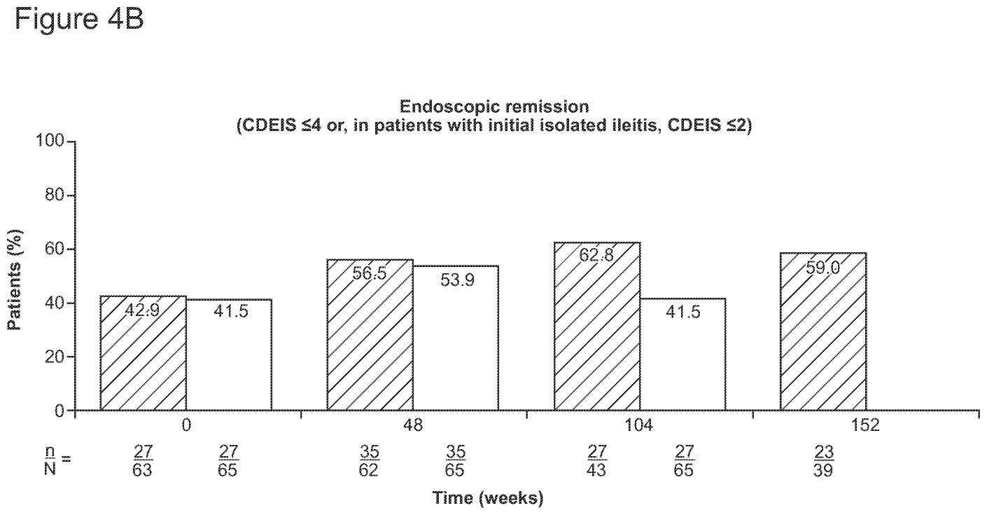

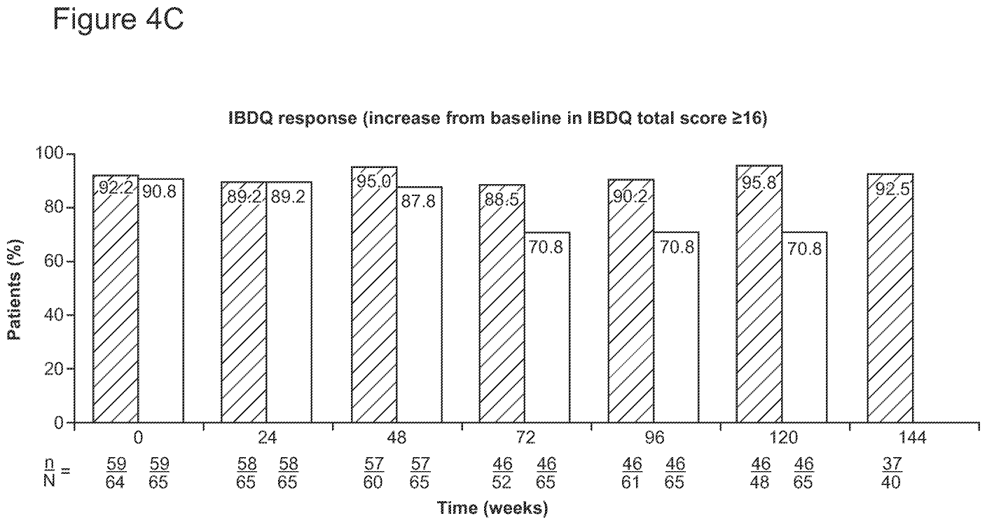

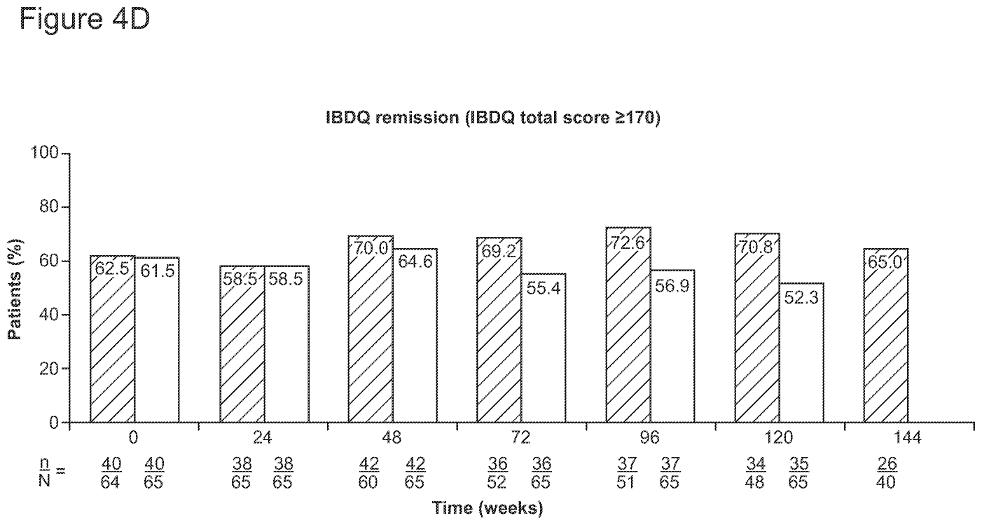

The proportions of patients in clinical remission and endoscopic remission were maintained with long-term risankizumab treatment throughout the duration of the study. Central reviewer scoring was used for all endoscopic efficacy assessments. Clinical remission was achieved in >71% patients in the observed analysis and >64% patients in the NRI analysis at any given visit through week 112 of OLE therapy (FIG. 4A). Endoscopic remission was achieved in >42% patients in the observed analysis and >41% patients in the NRI analysis at any given visit through week 104 (FIG. 4B).

A high proportion of patients reported HRQoL improvements from baseline (prior to the first dose of risankizumab in the M15-993 study), assessed by IBDQ response (>88% patients in the observed analysis and >70% patients in the NRI analysis; FIG. 4C) and IBDQ remission (>58% patients in the observed analysis and >52% patients in the NRI analysis; FIG. 4D) at any given visit through week 120. These improvements were observed across all four domains of IBDQ (bowel symptoms, systemic symptoms, social function, and emotional function).

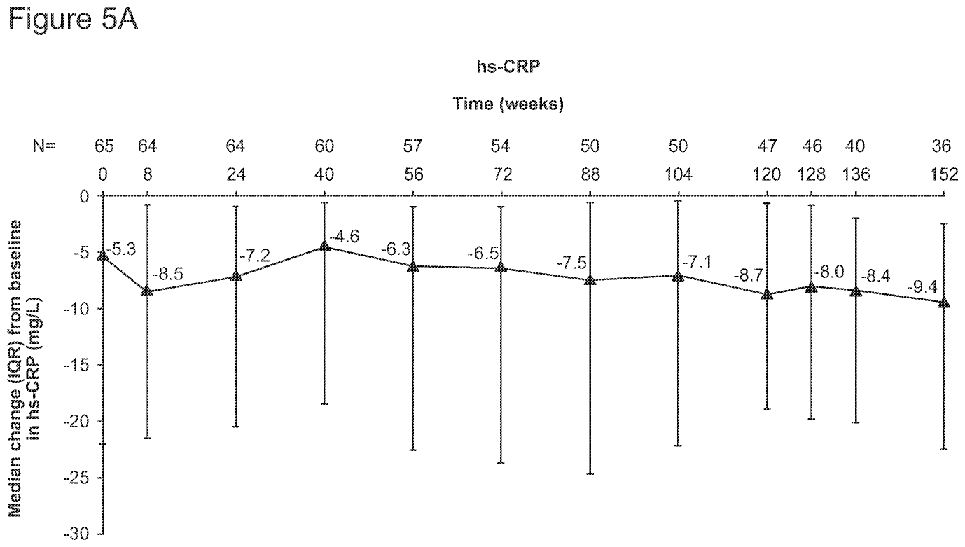

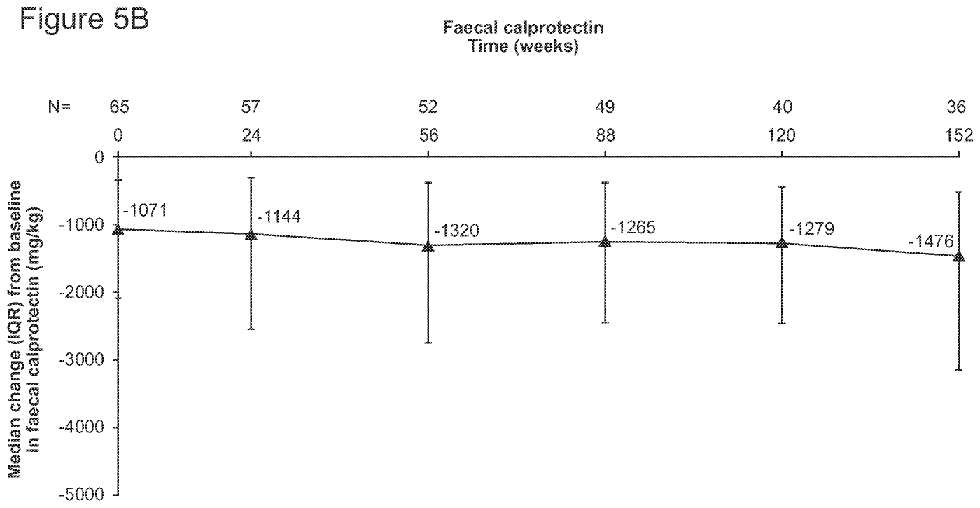

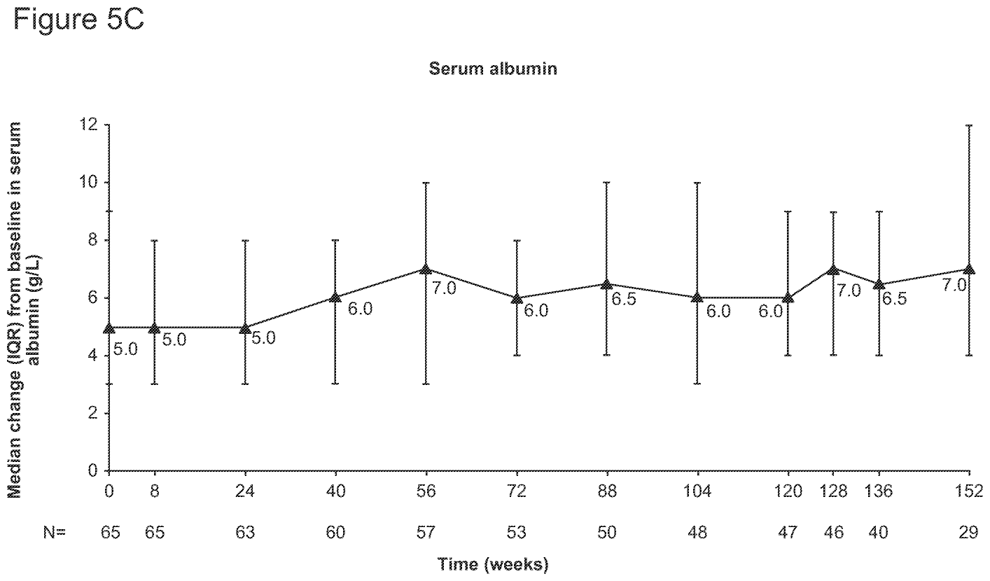

Median reductions from baseline (prior to the first dose of risankizumab in the M15-993 study) in hs-CRP and faecal calprotectin observed at week 0 of this OLE were generally maintained over the course of the OLE through week 152 (FIGS. 5A and 5B). Increased serum albumin levels at week 0 were also generally maintained through week 152 (FIG. 5C).

In this OLE, risankizumab 180 mg SC Q8W maintenance treatment was well tolerated by patients with CD with over 3 years and 167 PY of treatment, and no new safety signals were observed. As secondary outcomes, the pharmacokinetics of risankizumab were consistent with previous observations (Feagan B G et al. (2018) Lancet Gastroenterol. Hepatol. 3:671-80; Suleiman et al. (2019) Clin. Pharmacokinet. 58:375-87), and efficacy was maintained during the study. Patients included in this study had treatment-refractory CD at M15-993 baseline and completed both the M15-993 and M15-989 studies before rolling over to the phase 3 OLE, suggesting that the safety and efficacy of risankizumab were compelling enough to warrant continued risankizumab.

The majority of SAEs were gastrointestinal in nature and may reflect underlying CD. Rates of serious infections, opportunistic infections, and fungal infections in the OLE (4.2, 1.8, and 6.6 E/100 PY, respectively) were similar to those reported in the parent phase 2 M15-993 study (4.6, 2.7, and 7.3 E/100 PY, respectively) (Feagan B G et al. (2018) Lancet Gastroenterol. Hepatol. 3:671-80). Serious infections were predominantly driven by gastrointestinal events, and no cases of tuberculosis (including latent tuberculosis) or herpes zoster were reported. Hypersensitivity reactions were observed in about 25% patients and occurred at a lower rate than that observed in the M15-993 study. There was one serious anaphylactic reaction to IV iron carboxymaltose administration that did not lead to drug discontinuation. No deaths, malignancies, or adjudicated cardiovascular events were reported.

Laboratory value [symbol] Grade 3 were infrequent and usually transient. The safety profile of risankizumab 180 mg in patients with CD was generally consistent with previous reports in psoriasis and psoriatic arthritis, despite the difference in risankizumab doses evaluated across indications (150 mg every 12 weeks for psoriasis and 150 mg every 4 or 12 weeks for psoriatic arthritis) (Gordon et al. (2018) Lancet. 392:650-61; Reich et al. (2019) Lancet. 394:576-86; Mease et al. (2018) Ann. Rheum. Dis. 77:200-1). The types of TEAE reported with risankizumab in patients with CD were similar to those reported by studies of ustekinumab, an IL-12/23 inhibitor (Feagan et al. (2016) N. Engl. J. Med. 375:1946-60; Sandborn et al. (2018) Aliment, Pharmacol, Ther, 48:65-77; Ghosh et al. (2019) Drug Saf. 42:751-68).

Steady-state plasma exposure was achieved by week 24 following administration of risankizumab 180 mg SC Q8W. The development of largely low-titre, non-neutralising ADAs in eight patients did not appear to have a clear impact on risankizumab plasma exposures. These results are consistent with previous studies of psoriasis, in which only high-titre ADAs (>128) for risankizumab had a meaningful impact on pharmacokinetics (Khatri et al. (2020) Clin. Pharmacol. Ther. 107:378-87; Pang et al. (2020) Clin. Pharmacokinet. 59:311-26). Therefore, no marked impact on risankizumab exposure, and thus efficacy and safety, was expected given that the majority of the ADAs in this study were of low titre. Although four out of the eight patients who developed treatment-emergent ADAs discontinued from the study, based on review of the time-course data for ADA titres, risankizumab exposure, and reasons for discontinuation there was no apparent correlation between immunogenicity and discontinuation.

In this OLE, maintenance treatment with risankizumab 180 mg SC Q8W for up to 184 weeks was well tolerated by patients with moderate-to-severe refractory CD, and no new safety signals were observed. Results from ongoing phase 3 studies, including the phase 3 OLE, further inform the efficacy and safety of risankizumab in patients with CD.

### Example 3: Protocol of the Phase 3 Induction Study M15-991

M15-991 was a multicenter, randomized, double-blind, placebo-controlled induction study to assess the efficacy and safety of risankizumab in subjects with moderately to severely active Crohn's disease who failed prior biologic treatment.

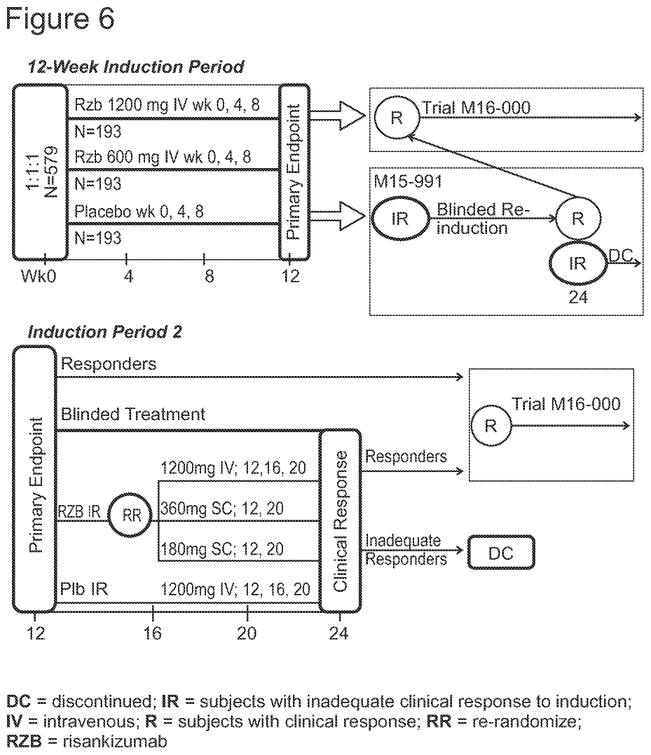

Objectives: The objectives of Study M15-991 (FIG. 6) included evaluating the efficacy and safety of risankizumab versus placebo during induction therapy in subjects with moderately to severely active Crohn's disease (CD). One of the primary objectives was to evaluate the efficacy and safety of RISA compared to PBO (placebo) in inducing clinical remission and endoscopic response in subjects with moderately to severely active CD who demonstrated intolerance or inadequate response to use of an approved biologic agent (Bio-IR), such as infliximab, adalimumab, certolizumab, natalizumab, ustekinumab and/or vedolizumab.

Investigators: Global multicenter

Study Sites: Approximately 400 sites.

Study Population: Males and females aged [symbol] 18 to [symbol] 80 years of age, or minimum age of adult consent according to local regulations at the Baseline visit, or aged 16 to <18 years of age where locally permitted and who met the definition of Tanner stage 5 development, at the Baseline visit, with a diagnosis of moderately to severely active CD, defined as:

1. average daily stool frequency (SF) [symbol] 4 (when calculating SF, only the number of liquid or very soft stools should be recorded) and/or average daily abdominal pain (AP) score [symbol] 2; plus

2. endoscopic evidence of mucosal inflammation measured by the Simple Endoscopic Score for CD (SES-CD). All eligible scores exclude the presence of narrowing component and are confirmed by a central reader. Endoscopic activity is defined as a SES-CD of [symbol] 3.

The number of subjects enrolled with a SES-CD of [symbol] 3 to <6 for ileocolonic or colonic disease or SES-CD of 3 for isolated ileal disease was no more than 58 subjects. Once cap of no more than 58 subjects was reached, enrollment criterion was an eligibility SES-CD of [symbol] 6 for ileocolonic or colonic disease, or eligibility SES-CD of [symbol] 4 for isolated ileal disease.

The study enrolled 100% of subjects who had an inadequate response or intolerance to prior biologic therapy (bio-IR). The bio-IR population was defined as subjects with documented intolerance or inadequate response to one or more of the approved biologic agents for CD (infliximab, adalimumab, certolizumab, natalizumab, ustekinumab and/or vedolizumab). The percent of subjects with exposure, including intolerance or inadequate response to ustekinumab was no more than 20%.

Number of Subjects to be Enrolled: Approximately 579 for the primary ITT population used for efficacy analysis; additionally, up to 58 subjects with lower SES-CD were enrolled.

Methodology:

Study M15-991 was a randomized, double blind, placebo-controlled 12-week induction study. Subjects (n=579) were randomized 1:1:1 to 1200 mg risankizumab or 600 mg risankizumab or placebo intravenous (IV) given at Baseline, Weeks 4, 8. The randomization was stratified by both the number of prior biologics failed (1, >1), Baseline steroid use (yes, no), and Baseline SES-CD (original, alternative), where the stratum of “original” included the patients with baseline SES-CD of [symbol] 6 (or [symbol] 4 for subjects with isolated ileal disease), and the stratum of “alternative” includes the patients with baseline SES-CD of [symbol] 3 to <6 for ileocolonic or colonic disease or SES-CD of 3 for isolated ileal disease.

Visits during the study occurred at Baseline and Weeks 4, 8, and 12/Premature Discontinuation (PD) to collect clinical and laboratory assessments of disease activity. Subjects who did not achieve clinical response at Week 12 might be eligible for treatment with blinded risankizumab in Induction Period 2 with evaluation for clinical response at Week 24. All subjects were provided with a subject diary where they recorded CD related symptoms throughout the study. Subjects were also dispensed a patient information card during Screening. Additionally, subjects completed symptom, quality of life (QoL) and work productivity questionnaires throughout the study. Clinical labs including, but not limited to, urinalysis, chemistry and hematology, high-sensitivity C-reactive protein (hs-CRP), serum risankizumab concentrations, and serum anti-drug antibody (ADA) levels were collected throughout the study. In addition, stool samples for calprotectin analysis were collected and taken before starting bowel preparations for endoscopy. All endoscopies were evaluated using SES-CD and were confirmed by a central reader. Biopsy to confirm diagnosis (during Screening) or to rule out dysplasia/malignancy might be performed during the same time points as the endoscopy. Optional exploratory research samples might be taken during the study.

At the Week 12/PD visit, all subjects underwent an endoscopy for evaluation of mucosal inflammation. It was expected that all subjects who remained in the study through at least Week 8 have a Week 12/PD endoscopy. All subjects achieving clinical response, defined as [symbol] 30% decrease in average daily SF and/or [symbol] 30% decrease in average daily AP score (both not worse than Baseline) at Week 12 might be eligible to enter Study M16-000. Subjects were not eligible to enter Study M16-000 until endoscopy had been completed (local reader results were used for stratification for Study M16-000).

All subjects who did not achieve clinical response at Week 12 might be eligible to receive blinded treatment with risankizumab in Induction Period 2 as described below. Subjects were not eligible to enter Induction Period 2 until Week 12 endoscopy had been completed.

Induction Period 2

At Week 12, subjects who did not achieve clinical response were randomized by Interactive Response Technologies (IRT) to Induction Period 2, a double-blind, double-dummy 12-week treatment period. Subjects who received IV risankizumab induction treatment were randomized 1:1:1 to:
Group 1: 1200 mg IV risankizumab
Group 2: 360 mg SC risankizumab
Group 3:180 mg SC risankizumab

Subjects who received placebo induction treatment received:
Group 4:1200 mg IV risankizumab

The IV risankizumab dose or matching IV placebo was given at Weeks 12, 16, and 20; The SC risankizumab dose or matching SC placebo was given at Weeks 12, and 20. At Week 24, subjects who received treatment in Induction Period 2 were reassessed and underwent a third endoscopy for evaluation of mucosal inflammation. Subjects who achieved clinical response at Week 24 may be eligible to enter Study M16-000. Subjects without clinical response at Week 24, as well as all subjects who terminated the study early (including subjects who are eligible for, but do not receive treatment during Induction Period 2) were discontinued and have a follow-up call 140 days from the last dose of study drug to obtain information on any new or ongoing AEs.

Diagnosis and Main Criteria for Inclusion/Exclusion:

Main Inclusion:

1. Male or female aged [symbol] 18 to [symbol] 80 years of age, or minimum age of adult consent according to local regulations, at the Baseline visit. Where locally permissible, subjects 16 to <18 years of age who meet the definition of Tanner stage 5 for development (refer to Appendix 1), at the Baseline Visit (sites are notified when adolescents may enroll).

2. Confirmed diagnosis of CD for at least 3 months prior to Baseline. Appropriate documentation of biopsy results consistent with the diagnosis of CD, in the assessment of the Investigator, must be available.

3. Crohn's disease activity index (CDAI) score 220-450 at Baseline.

4. Endoscopic evidence of mucosal inflammation as documented by an SES-CD of [symbol] 3. All eligible scores exclude the presence of narrowing component and are confirmed by a central reader. (Once cap of no more than 58 subjects is reached, enrollment criterion is an SES-CD of [symbol] 6 for ileocolonic or colonic disease or SES-CD of [symbol] 4 for isolated ileal disease.)

5. Average daily SF [symbol] 4 and/or average daily AP score [symbol] 2 at Baseline.

6. Demonstrated intolerance or inadequate response to one or more of the following biologic agents: infliximab, adalimumab, certolizumab pegol, natalizumab, ustekinumab and/or vedolizumab:
Demonstration of intolerance requires no minimum dose or duration of use;
Inadequate response to biologic agents defined as signs and symptoms of persistently (in the opinion of the Investigator) active disease despite a history of one or more of the following:
   At least one 6-week induction regimen of infliximab ( [symbol] 5 mg/kg IV at Weeks 0, 2, and 6), [symbol]
   At least one 4-week induction regimen of adalimumab (one 160 mg SC dose at Week 0, followed by one 80 mg SC dose at Week 2 [or one 80 mg SC dose at Week 0, followed by one 40 mg SC dose at Week 2, in countries where this dosing regimen is approved]),
   At least one 4-week induction regimen of certolizumab pegol (400 mg SC at Weeks 0, 2, and 4)
   At least one 6-week induction regimen of vedolizumab (300 mg IV at Weeks 0, 2, and 6)
   At least one 12-week induction regimen of natalizumab (300 mg IV every 4 weeks)
   At least one 8-week induction regimen of ustekinumab [260 mg ( [symbol] 55 kg) or 390 mg (>55 to [symbol] 85 kg) or 520 mg (>85 kg) IV, followed by 90 mg SC at Week 8] (Once cap of no more than 20% ustekinumab exposed subjects is reached, subjects with prior ustekinumab exposure are not allowed to enroll.)
Recurrence of symptoms during scheduled maintenance dosing following prior clinical benefit of the above biologic agents
Note: Subjects who discontinued biologic agents for reasons other than inadequate response as defined above or intolerance (e.g., change of insurance) are not eligible to enroll

7. If female, subject must be either postmenopausal or permanently surgically sterile, or for women of childbearing potential, practicing birth control, prior to Baseline through at least 140 days after the last dose of study drug. Females of childbearing potential must have a negative serum pregnancy test result during Screening, and a negative urine pregnancy at Baseline. Females of non-childbearing potential (either postmenopausal or permanently surgically sterile) during Screening do not require pregnancy testing at Baseline. Note: Subjects with borderline serum pregnancy test at Screening must have a serum pregnancy test [symbol] 3 days later to document continued lack of a positive result.

[symbol]

8. Subjects must be able and willing to give written informed consent and to comply with the requirements of this study protocol.

Main Exclusion:

1. Subject with a current diagnosis of ulcerative colitis or indeterminate colitis.

Concomitant Medications and Treatments

2. Subject on CD-related antibiotics who has not been on stable doses for greater than, or discontinued within, 14 days prior to Baseline.

3. Subject on oral aminosalicylates who has not been on stable doses for greater than, or discontinued within, at least 14 days prior to Baseline.

4. Subject taking oral corticosteroids:
Budesonide >9 mg/day
Beclomethasone >5 mg/day
Prednisone or equivalent >20 mg/day
Or has not been on the current course for [symbol] 14 days prior to Baseline and on a stable dose for [symbol] 7 days prior to Baseline

5. Subject on immunomodulators (AZA, 6-MP, MTX) who:
Has not been on the current course for [symbol] 42 days prior to Baseline, and
Has not been on a stable dose for [symbol] 35 days prior to Baseline

Medications and Treatments During the Screening Period

6. Subject who received IV anti-infectives within 35 days prior to Baseline visit or oral/intramuscular anti-infectives (non-CD-related) within 14 days prior to the Baseline visit. This does not apply to TB prophylaxis.

7. Subject who received exclusive enteral nutrition or any parenteral nutrition within 35 days prior to Baseline.

8. Subject who received any live bacterial or viral vaccination within 30 days prior to Screening or during the Screening period.

9. Subject who received cyclosporine, tacrolimus, or mycophenolate mofetil within 35 days prior to Baseline.

10. Subject who received fecal microbial transplantation within 35 days prior to Baseline.

Prior Medications and Treatments

11. Subject who received any biologic agent:
Approved biologics: infliximab, adalimumab, certolizumab, natalizumab, vedolizumab within 8 weeks prior to Baseline, or ustekinumab within 12 weeks prior to Baseline. Note: If there is proper documentation of an undetectable drug level measured by a commercially available assay for any of the approved biologics above, there is no minimum washout prior to Baseline.
Any investigational biologic or other agent or procedure within 35 days or 5 half-lives prior to Baseline, whichever is longer.

12. Subject with prior exposure to p19 inhibitors (e.g., risankizumab).

13. Subject has been taking combination of two or more of the following oral budesonide, or oral beclomethasone and/or oral prednisone (or equivalent) simultaneously, with the exception of inhalers, within 14 days prior to Screening or during the Screening period.

14. Subject who received IV/intramuscular corticosteroids within 14 days prior to Screening or during the Screening period.

15. Subject who received therapeutic enema or suppository, other than required for endoscopy, within 14 days prior to endoscopy used for Screening or during the Screening period.

16. Subject who received apheresis (e.g., Adacolumn apheresis) ≤60 days prior to Screening or during the Screening period.

17. Subject who has concomitant cannabis use either recreational or for medical reasons within 14 days of Baseline or any history of clinically significant drug, or alcohol abuse in the last 12 months.

CD Related

18. Subject with currently known complications of CD such as:
abscess (abdominal or perianal),
symptomatic bowel strictures,
>2 missing segments of the following 5 segments: terminal ileum, right colon, transverse
colon, sigmoid and left colon, and rectum
fulminant colitis,
toxic megacolon,
or any other manifestation that might require surgery while enrolled in the study.

19. Subject with ostomy or ileoanal pouch.

20. Subject diagnosed with short gut or short bowel syndrome.

21. Subject with surgical bowel resection within the past 3 months prior to Baseline, or a history of [symbol] 3 bowel resections.

22. Subject who has a known hypersensitivity to risankizumab or the excipients of any of the study drugs or the ingredients of Chinese hamster ovary (CHO).

23. Subjects with the following chronic or active infections:
Active, chronic, or recurrent infection that based on the Investigator's clinical assessment makes the subject unsuitable candidate for the study,
Infection with C. difficile toxin or other intestinal pathogen during Screening,
Are infected with human immunodeficiency virus (HIV),
QuantiFERON®-TB test or Purified Protein Derivative (PPD) skin test, or both, according to local guidelines, are performed during Screening. QuantiFERON®-TB test is preferred for subjects who received BCG vaccination or were exposed to other Mycobacteria species. Subjects with a positive test result (or indeterminate results that have been repeated) may participate in the study if further work up (according to local practice/guidelines) establishes conclusively that the subject has no evidence of active tuberculosis. Subjects with a history of active TB who have documented completion of a full course of anti-TB therapy may be allowed to enter the study after consultation with the AbbVie TA MD. If latent TB is established, TB prophylaxis/treatment should be initiated and maintained according to local country guidelines.
Have active hepatitis B or hepatitis C defined as:
   HBV: hepatitis B surface antigen (HBs Ag) positive (+), or detected sensitivity on the HBV deoxyribonucleic acid (DNA) polymerase chain reaction (PCR) qualitative test for hepatitis B core antibody (HBc Ab) positive subjects
   HCV: HCV ribonucleic acid (RNA) detectable in any subject with positive anti-HCV antibody (HCV Ab)

24. Subject with a previous history of dysplasia of the gastrointestinal tract or found to have dysplasia, other than completely removed low-grade dysplastic lesions, in any biopsy performed during the Screening endoscopy.

25. Subject with a known history of lymphoproliferative disease, including lymphoma, or signs and symptoms suggestive of possible lymphoproliferative disease, such as lymphadenopathy and/or splenomegaly.

26. Subject with current or previous history of malignancy other than a successfully treated non-metastatic cutaneous squamous cell or basal cell carcinoma or localized carcinoma in situ of the cervix.

27. Subject who has severe, progressive, or uncontrolled renal, hepatic, hematological, endocrine, disorder or symptoms thereof.

28. Female subjects who is pregnant, breastfeeding, or is considering becoming pregnant during the study or for approximately 140 days after the last dose of study drug.

29. Subject who has any condition, including any physical, psychological, or psychiatric condition, which in the opinion of the Investigator, would compromise the safety of the subject or the quality of the data and renders the subject an unsuitable candidate for the study.

30. Screening laboratory and other analyses show any of the following abnormal results:
Aspartate transaminase (AST), alanine transaminase (ALT) >2×upper limit of the reference range;
White blood cell (WBC) count <3.0×109/L;
Total bilirubin [symbol] 2 mg/dL; except for subjects with isolated elevation of indirect bilirubin relating to Gilbert syndrome;
Estimated glomerular filtration rate by simplified 4-variable Modification of Diet in Renal Disease (MDRD) formula <30 ml/min/1.73 m2.
Hemoglobin <8 g/dL
Platelets <100,000/μL
Positive serum pregnancy test at the Screening visit or positive urine pregnancy test at the Baseline visit

Laboratory values can be re-tested once during the screening period after discussion and clearance with the TAMD. If the re-tested lab value(s) remain(s) exclusionary, the subject is considered a screen failure. Redrawing samples if previous samples were unable to be analyzed would not count as a retest since previous result was never obtained.

31. No known active COVID-19 infection. Subject must not have signs/symptoms associated with COVID-19 infection.

Subjects who do not meet COVID-19 eligibility criteria must be screen failed and may only rescreen after they meet the following COVID-19 viral clearance criteria:
Symptomatic subjects: At least 2 negative viral tests in a row completed locally, [symbol] 24 hours apart after at least 10 days have passed since recovery, defined as resolution of fever without use of antipyretics and improvement in respiratory symptoms (e.g., cough, shortness of breath)
Asymptomatic subjects: At least 2 negative viral tests in a row completed locally, [symbol] 24 hours apart after at least 10 days have passed since prior positive result (Note: subjects who develop symptoms follow guidance above for symptomatic subjects)

Frequency or timing of COVID-19 testing and interval between testing for the above viral clearance criteria might be adjusted to account for epidemiological trends, updated information regarding infectivity and local/institutional guidelines.

Investigational Product:
Risankizumab

Dose(s):
Risankizumab 1200 mg IV Q4W*
Risankizumab 600 mg IV Q4W
Risankizumab 180 mg SC Q8W
Risankizumab 360 mg SC Q8W *If administration of the 1200 mg dose was halted for any reason, enrollment might continue for subjects into the risankizumab 600 mg, or placebo arms at a 1:1 ratio. The ratio of randomization and final sample size might be further updated in an amendment to the protocol. Subjects already randomized to treatment arms, including Induction Period 2, received blinded 600 mg risankizumab IV.

Mode of Administration:
Risankizumab solution for infusion (IV)
Risankizumab solution for injection (SC)

Reference Therapy: Placebo for risankizumab

Dose(s): Placebo: N/A

Mode of Administration:
Placebo solution for infusion (IV)
Placebo solution for injection (SC)

Duration of Treatment: 12 or 24 weeks.

The study included a Screening period of 35 days and a double-blind induction period of 12 weeks. All subjects who did not achieve clinical response at Week 12 might be eligible to receive risankizumab in Induction Period 2 over a subsequent 12 week period. There was a follow up call 140 days from the last dose of study drug to obtain information on any new or ongoing AEs for those subjects who did not roll over into Study M16-000 or discontinue from the study prematurely.

Criteria for Evaluation:

Endpoint Definitions:
Clinical remission: average daily SF [symbol] 2.8 and not worse than Baseline AND average daily AP score [symbol] 1 and not worse than Baseline
Enhanced clinical response: [symbol] 60% decrease in average daily SF and/or [symbol] 35% decrease in average daily AP score and both not worse than Baseline, and/or clinical remission
Clinical response: [symbol] 30% decrease in average daily SF and/or [symbol] 30% decrease in average daily AP score and both not worse than Baseline
Endoscopic response: decrease in SES-CD >50% from Baseline (or for subjects with isolated ileal disease and a Baseline SES-CD of 4, at least a 2 point reduction from Baseline), as scored by central reviewer
Ulcer-free endoscopy: SES-CD ulcerated surface subscore of 0 in subjects with SES-CD ulcerated surface subscore [symbol] 1 at Baseline, as scored by a central reviewer.
Endoscopic remission: SES-CD [symbol] 4 and at least a 2 point reduction versus baseline and no subscore greater than 1 in any individual variable, as scored by a central reviewer
CDAI clinical response: reduction of CDAI [symbol] 100 points from baseline
CDAI clinical remission: CDAI <150
SF remission: average daily SF [symbol] 2.8 and not worse than baseline
AP remission: average daily AP score [symbol] 1 and not worse than baseline

Efficacy: The US-specific protocol and the global protocol outside US have used different co-primary endpoints and multiplicity-controlled secondary endpoints.

For US-Specific Protocol:

Co-Primary Endpoints
The achievement of clinical remission (CDAI) at Week 12
The achievement of endoscopic response at Week 12

Secondary Endpoints
1. The achievement of clinical remission (SF/APS) at Week 12
2. The achievement of CDAI clinical response at Week 4
3. The achievement of CDAI clinical response at Week 12
4. Change from baseline in Functional Assessment of Chronic Illness Therapy-Fatigue (FACIT-Fatigue) at Week 12
5. The achievement of clinical remission (CDAI) at Week 4
6. The achievement of CDAI clinical response and endoscopic response at Week 12
7. The achievement of SF remission at Week 12
8. The achievement of AP remission at Week 12
9. The achievement of endoscopic remission at Week 12
10. The achievement of enhanced clinical response at Week 4
11. The achievement of ulcer-free endoscopy at Week 12
12. The achievement of enhanced clinical response at Week 12
13. The achievement of resolution of extra-intestinal manifestations (EIMs) at Week 12, in subjects with any EIMs at baseline
14. Occurrence of CD-related hospitalization through Week 12
15. The achievement of no draining fistulas at Week 12 in subjects with draining fistulas at baseline

For global protocol outside US:

Co-Primary Endpoints
The achievement of clinical remission (SF/APS) at Week 12
The achievement of endoscopic response at Week 12

Secondary Endpoints
1. The achievement of clinical remission (CDAI) at Week 12
2. The achievement of CDAI clinical response at Week 4
3. The achievement of clinical remission (SF/APS) at Week 4
4. The achievement of CDAI clinical response at Week 12
5. Change from baseline in FACIT-Fatigue at Week 12
6. Change from baseline in Inflammatory Bowel Disease Questionnaire (IBDQ) total score at Week 12
7. The achievement of enhanced clinical response and endoscopic response at Week 12
8. The achievement of endoscopic remission at Week 12
9. The achievement of enhanced clinical response at Week 4
10. The achievement of ulcer-free endoscopy at Week 12
11. The achievement of enhanced clinical response at Week 12
12. The achievement of resolution of EIMs at Week 12, in subjects with any EIMs at baseline
13. Occurrence of CD-related hospitalization through Week 12
14. The achievement of no draining fistulas at Week 12 in subjects with draining fistulas at baseline
15. Change from baseline in Work Productivity and Impairment Questionnaire-Crohn's disease (WPAI-CD) Overall Work Impairment at Week 12
16. Change from baseline in Short Form-36 (SF-36) Physical Component Summary score at Week 12

Pharmacokinetics:

Serum risankizumab concentrations were determined from samples collected just prior to dosing at Weeks 4, 8, and 12/PD, and at Week 24 for subjects who entered Induction Period 2.

Immunogenicity:

Serum ADAs were determined from samples collected just prior to dosing at Baseline and Weeks 4, 8, and 12/PD, and at Week 24 for subjects who entered Induction Period 2.

Safety:

Safety analyses were performed on safety set which includes all subjects who received at least one dose of study drug. Incidence of adverse events (AEs), changes in vital signs, physical examination results, and clinical laboratory data were assessed throughout the study.

Statistical Methods:

Efficacy:

The co-primary endpoints were the proportion of subjects with CDAI clinical remission at Week 12 and the proportion of subjects who achieved endoscopic response at Week 12. A total of approximately 579 subjects were randomized into two risankizumab treatment groups and the placebo group in a 1:1:1 ratio (193 subjects/arm). When all patients completed their Week 12/PD visit, the database was locked for final analysis of 12-week induction period. When all the patients who entered the induction Period 2 finished Week 24/PD visit, the database was locked for the whole study and all the planned analysis for the induction Period 2 was performed.

Assuming the Week 12 CDAI clinical remission rate is 34% for one of the risankizumab groups and 15% for the placebo group, a sample size of 193 subjects for each risankizumab group and 193 subjects for the placebo group has approximately 97% power to detect the treatment difference between risankizumab dose groups and placebo in CDAI clinical remission rates at Week 12 using Fisher's exact test at a 0.025 significant level (two sided).

The comparisons between each risankizumab dose versus placebo for the primary efficacy variable was performed using the Cochran-Mantel-Haenszel (CMH) test adjusted by the number of prior biologics failed (1, >1) and Baseline steroid use (yes, no).

Both of the primary efficacy endpoints were tested at statistically significant of 0.025 for each of the risankizumab dose group versus placebo to adjust for multiplicity. A CMH based two-sided 95% confidence interval for the difference between treatment groups was calculated.

The intent-to-treat (ITT) set included all randomized subjects who had taken at least one dose of study drug. The primary population for efficacy analysis were the subjects in the intent-to-treat analysis set who had baseline eligibility SES-CD of [symbol] 6 ( [symbol] 4 for isolated ileal disease). Subjects who discontinued prior to Week 12 for any reason was considered as “not-achieved” for CDAI clinical remission and endoscopic response endpoints.

In general, continuous secondary efficacy variables were analyzed using a Mixed-Effect Model Repeated Measure (MMRM) model including factors for treatment group, visit, visit by treatment interaction, and stratification variables, for the longitudinal continuous endpoints. The MMRM analysis was considered primary for inferential purposes.

Pharmacokinetics and Immunogenicity:

Serum risankizumab concentrations were summarized at each time point for each dosing regimen using descriptive statistics. Population pharmacokinetic analyses combining the data from this study and other studies might be performed. Relationships between risankizumab exposures and efficacy and safety variables of interest might be explored.

ADA incidence was summarized by cohorts and study visits. ADA titers were tabulated for each subject at the respective study visits. The effect of ADA on risankizumab pharmacokinetics, efficacy and/or safety variable(s) and/or any additional analyses was explored.

Safety:

Adverse events, laboratory data and vital signs were the primary safety parameters in this study. All safety comparisons were performed between treatment groups using the safety set. Treatment emergent AEs were defined as events that begin or worsen either on or after the first dose of the study drug and within 140 days after the last dose of the study drug for subjects who did not participate in Study M16-000 or until first dose of study drug in Study M16-000 if the subject was enrolled in Study M16-000.

An overview of treatment-emergent AEs, AEs leading to death and AEs leading to premature discontinuation (see details in the statistical analysis plan [SAP]), AEs by Medical Dictionary for Drug Regulatory Activities (MedDRA version) preferred term and system organ class, AEs by maximum relationship to study drug, and AEs by maximum severity were summarized by number and percentage.

Changes in laboratory data were described using statistical characteristics and compared between-treatment groups is performed using a one-way Analysis of Variance (ANOVA). In addition, shift tables and listings were provided for abnormal values, whereby the normal range of the analyzing laboratory was used. Vital signs were analyzed similarly.

### Example 4: Protocol of the Phase 3 Induction Study M16-006

M16-006 was a global (US and outside-US) multicenter, randomized, double-blind, placebo-controlled induction study of the efficacy and safety of risankizumab in subjects with moderately to severely active Crohn's disease.

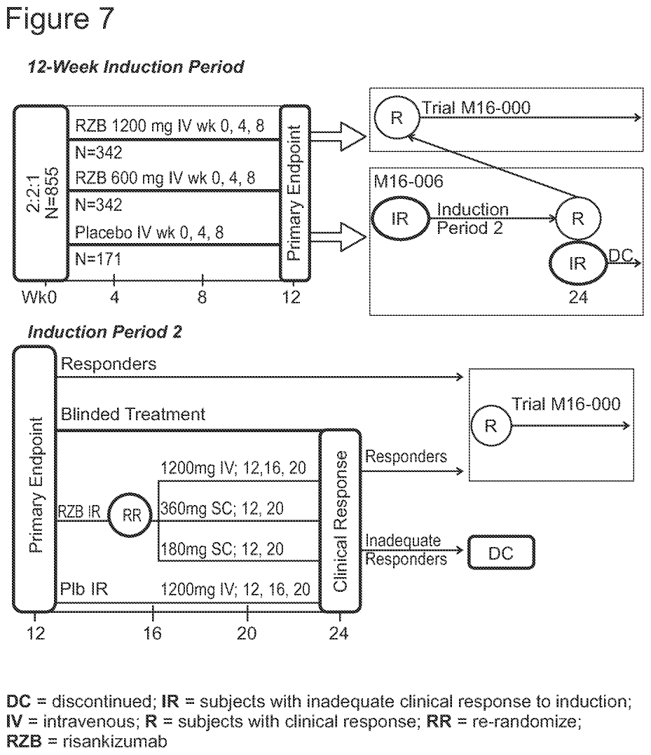

Objective: The objective of Study M16-006 (FIG. 7) was to evaluate the efficacy and safety of risankizumab versus placebo during induction therapy in subjects with moderately to severely active Crohn's disease (CD).

Investigators: Multicenter

Study Sites: Approximately 400 sites worldwide

Study Population: Males and females aged [symbol] 18 to [symbol] 80 years of age, or minimum age of adult consent according to local regulations at the Baseline visit, or aged 16 to <18 years of age where locally permitted and who meet the definition of Tanner stage 5 development at the Baseline visit, with a diagnosis of moderately to severely active CD, defined as:

1. average daily stool frequency (SF) [symbol] 4 (when calculating SF, only the number of liquid or very soft stools should be recorded) and/or average daily abdominal pain (AP) score [symbol] 2; plus

2. endoscopic evidence of mucosal inflammation as measured by the Simple Endoscopic Score for CD (SES-CD). All eligible scores excluded the presence of narrowing component and are confirmed by a central reader. Endoscopic activity was defined as a SES-CD of [symbol] 3.

Moderately to severely active CD may also be defined as meeting the following: 1) CDAI of 220 to 450; and 2) average daily stool frequency (SF) [symbol] 4 and/or average daily abdominal pain (AP) score [symbol] 2.

The number of subjects enrolled with a SES-CD of [symbol] 3 to <6 for ileocolonic or colonic disease or SES-CD of 3 for isolated ileal disease was no more than 85 subjects. Once cap of no more than 85 subjects was reached, enrollment criterion was an eligibility SES-CD of [symbol] 6 for ileocolonic or colonic disease, or eligibility SES-CD of [symbol] 4 for isolated ileal disease.

The study enrolled both subjects who have had an inadequate response (IR) to prior biologic therapy (bio-IR) and subjects who have not (non-bio-IR). The bio-IR enrollment was approximately 540 subjects and the non-bio-IR enrollment was approximately 315 subjects. The bio-IR population was defined as subjects with documented intolerance or inadequate response to one or more of the approved anti-TNF or anti-integrin biologics for CD (infliximab, adalimumab, certolizumab, vedolizumab, ustekinumab and/or natalizumab).

The non-bio-IR population included subjects who had an inadequate response or intolerance to conventional therapy. Conventional therapy was defined as one or more of the following: aminosalicylates, oral locally acting steroids (e.g., budesonide, beclomethasone), systemic corticosteroids (prednisone or equivalent), or immunomodulators. This population also included subjects who have received biologic therapy in the past but stopped therapy based on reasons other than inadequate response or intolerance (e.g., change in reimbursement coverage, well-controlled disease).

The percent of subjects with exposure, including intolerance or inadequate response, to ustekinumab was no more than 20%.

Number of Subjects to be Enrolled: Approximately 855 for the primary ITT population used for efficacy analysis; additionally, up to 85 subjects with lower SES-CD were enrolled.

Methodology:

Study M16-006 was a randomized, double blind, placebo-controlled 12-week induction study. Subjects (n=855) were randomized 2:2:1 to 1200 mg risankizumab or 600 mg risankizumab or placebo intravenous (IV) given at Baseline, Weeks 4 and 8. The randomization was stratified by number of prior biologics failed (0, 1, >1), Baseline steroid use (yes, no), and Baseline SES-CD (original, alternative), where the stratum of “original” includes the patients with baseline SES-CD of [symbol] 6 (or [symbol] 4 for subjects with isolated ileal disease), and the stratum of “alternative” included the patients with baseline SES-CD of [symbol] 3 to <6 for ileocolonic or colonic disease or SES-CD of 3 for isolated ileal disease.

Visits during the study occurred at Baseline and Weeks 4, 8, and 12/Premature Discontinuation (PD) to collect clinical and laboratory assessments of disease activity. Subjects who did not achieve clinical response at Week 12 were offered blinded risankizumab therapy in Induction Period 2 with evaluation for clinical response at Week 24.

All subjects were provided with a subject diary where they recorded CD related symptoms throughout the study. Subjects were also dispensed a patient information card at Screening. Additionally, subjects completed symptom, quality of life (QoL) and work productivity questionnaires throughout the study. Clinical labs including, but not limited to, urinalysis, chemistry and hematology, high-sensitivity C-reactive protein (hs-CRP), serum risankizumab concentrations, and serum anti-drug antibody (ADA) levels were collected throughout the study. In addition, stool samples for calprotectin analysis was collected and taken before starting bowel preparations for endoscopy. All endoscopies were evaluated using SES-CD and were confirmed by a central reader. Biopsy to confirm diagnosis (during Screening) or to rule out dysplasia/malignancy may be performed during the same time points as the endoscopy. Optional exploratory research samples may be taken during the study.

At the Week 12/PD visit, all subjects underwent an endoscopy for evaluation of mucosal inflammation. It was expected that all subjects who remained in the study through at least Week 8 would have a Week 12/PD endoscopy. All subjects achieving clinical response, defined as [symbol] 30% decrease in average daily SF and/or [symbol] 30% decrease in average daily AP score (both not worse than Baseline) at Week 12 may be eligible to enter Study M16-000. Subjects were not eligible to enter Study M16-000 until endoscopy was completed (local reader results were used for stratification for Study M16-000).

All subjects who did not achieve clinical response at Week 12 were eligible to receive blinded risankizumab treatment in Induction Period 2 as specified below. Subjects were not eligible to enter Induction Period 2 until the Week 12 endoscopy had been completed.

Induction Period 2:

At Week 12, subjects who do not achieve clinical response were randomized by Interactive Response Technologies (IRT) to Induction Period 2, a double-blind, double-dummy 12-week treatment period. Subjects who received IV risankizumab induction treatment with inadequate clinical response at Week 12 were randomized 1:1:1 to:
Group 1: 1200 mg IV risankizumab
Group 2: 360 mg SC risankizumab
Group 3:180 mg SC risankizumab

Subjects who received IV placebo induction treatment received:
Group 4:1200 mg IV risankizumab

The IV risankizumab dose or matching IV placebo was given at Weeks 12, 16, and 20. The SC risankizumab dose or matching SC placebo was given at Weeks 12, and 20. At Week 24, subjects who received treatment in Induction Period 2 were reassessed and underwent a third endoscopy for evaluation of mucosal inflammation. Subjects who achieved clinical response at Week 24 may have been eligible to enter Study M16-000. Subjects without clinical response at Week 24, as well as all subjects who terminated the study early (including subjects who were eligible for, but did not enter Induction Period 2), were discontinued and had a follow-up call 140 days from the last dose of study drug to obtain information on any new or ongoing AEs.

Diagnosis and Main Criteria for Inclusion/Exclusion:

Main Inclusion:

1. Males or females [symbol] 18 and [symbol] 80 years of age or minimum age of adult consent according to local regulations at the Baseline Visit. Where locally permissible, subjects 16 to <18 years of age who met the definition of Tanner stage 5 for development, at the Baseline Visit (sites were notified when adolescents may enroll).

2. Confirmed diagnosis of CD for at least 3 months prior to Baseline. Appropriate documentation of biopsy results consistent with the diagnosis of CD, in the assessment of the Investigator, must be available.

3. Crohn's disease activity index (CDAI) score 220-450 at Baseline.

4. Endoscopic evidence of mucosal inflammation as documented by an SES-CD of [symbol] 3. All eligible scores excluded the presence of narrowing component and were confirmed by a central reader. (Once cap of no more than 85 subjects was reached, enrollment criterion was an SES-CD of [symbol] 6 for ileocolonic or colonic disease or SES-CD of [symbol] 4 for isolated ileal disease.)

5. Average daily SF [symbol] 4 and/or average daily AP score [symbol] 2 at Baseline.

6. Demonstrated intolerance or inadequate response to one or more of the following categories of drugs: aminosalicylates, oral locally acting steroids, systemic steroids (prednisone or equivalent), immunomodulators, and/or biologic therapies
Demonstration of intolerance required no minimum dose or duration of use (intolerance included patients with a known TPMT genetic mutation or low activity).
Inadequate response is defined as outlined below:
   Oral aminosalicylates (e.g., mesalamine, sulfasalazine, olsalazine, balsalazide):
      Signs and symptoms of persistently active disease, in the opinion of the Investigator, during a current or prior course of at least 4 weeks of treatment with 2.4 g/day mesalamine, 4 g/day sulfasalazine, 1 g/day olsalazine, or 6.75 g/day balsalazide,
   Oral locally acting steroids (e.g., budesonide, beclomethasone):
      Signs and symptoms of persistently active disease, in the opinion of the Investigator, during or after a course of at least 4 weeks of treatment with 9 mg/day budesonide or 5 mg/day beclomethasone, or
      Inability to taper oral budesonide to at or below 6 mg/day without recurrent active disease,
   IV or Oral systemic steroids (prednisone or equivalent):
      Signs and symptoms of persistently active disease, in the opinion of the Investigator, during or after tapering of at least one regimen consisting of a dose equivalent to prednisone [symbol] 140 mg/day orally for 3 weeks or intravenously for 1 week, or [symbol]
      Inability to taper oral systemic steroids to at or below a dose equivalent to prednisone 10 mg/day without recurrent active disease,
   Immunomodulators:
      Signs and symptoms of persistently active disease, in the opinion of the Investigator, during a current or prior course of at least 90 days of treatment with one or more of the following:
         AZA: [symbol] 2.0 mg/kg/day rounded to the nearest available tablet or half tablet formulation ( [symbol] 1 mg/kg/day for subjects in Japan, Korea, Hong Kong, Taiwan, Singapore, or China) (or a documented 6-TGN level of [symbol] 230 pmol/8×108 RBC)
         6-MP: [symbol] 1 mg/kg/day rounded to the nearest available tablet or half tablet formulation ( [symbol] 0.6 mg/kg/day for subjects in Japan, Korea, Hong Kong, Taiwan, Singapore, or China) (or a 6-TGN level of [symbol] 230 pmol/8×108 RBC)
         MTX: [symbol] 15 mg/week subcutaneous (SC) or intramuscular (IM)
         Note: Oral MTX use was allowed during the study, however prior or current use of oral MTX is not sufficient for inclusion into the study
   Biologic Therapies for CD:
      Signs and symptoms of persistently (in the opinion of the Investigator) active disease despite a history of one or more of the following:
         At least one 6-week induction regimen of infliximab ( [symbol] 5 mg/kg IV at Weeks 0, 2, and 6),
         At least one 4-week induction regimen of adalimumab (one 160 mg SC dose at Week 0, followed by one 80 mg SC dose at Week 2 [or one 80 mg SC dose at Week 0, followed by one 40 mg SC dose at Week 2, in countries where this dosing regimen is approved]),
         At least one 4-week induction regimen of certolizumab pegol (400 mg SC at Weeks 0, 2, and 4),
         At least one 6-week induction regimen of vedolizumab (300 mg IV at Weeks 0, 2, and 6)
         At least one 12-week induction regimen of natalizumab (300 mg IV every 4 weeks)
         At least one 8-week induction regimen of ustekinumab [260 mg ( [symbol] 55 kg) or 390 mg (>55 to [symbol] 85 kg) or 520 mg (>85 kg) IV, followed by 90 mg SC at Week 8](Once cap of no more than 20% ustekinumab exposed subjects is reached, subjects with prior ustekinumab exposure were not allowed to enroll.)
      Recurrence of symptoms during scheduled maintenance dosing following prior clinical benefit of the above biologics
      Note: Subjects who discontinued biologics for reasons other than inadequate response as defined above or intolerance (e.g., change of insurance) must meet the criteria for intolerance or inadequate response to aminosalicylates, oral locally acting steroids, systemic steroids (prednisone or equivalent), and/or immunomodulators as defined above

7. If female, subject must be either postmenopausal or permanently surgically sterile, or for women of childbearing potential, practicing birth control, prior to Baseline through at least 140 days after the last dose of study drug. Females of childbearing potential must have a negative serum pregnancy test result during Screening, and a negative urine pregnancy at Baseline. Females of non-childbearing potential (either postmenopausal or permanently surgically sterile) during Screening do not require pregnancy testing at Baseline.

Note: Subjects with borderline serum pregnancy test at Screening must have a serum pregnancy test [symbol] 3 days later to document continued lack of a positive result.

8. Subjects must have been able and willing to give written informed consent and to comply with the requirements of this study protocol. In Japan, if the subject is <20 years old, a subject's parent or legal guardian must be willing to give written informed consent.

Main Exclusion:

1. Subject with a current diagnosis of ulcerative colitis or indeterminate colitis.

Concomitant Medications and Treatments

2. Subject on CD-related antibiotics who has not been on stable doses for greater than, or discontinued within, 14 days prior to Baseline.

3. Subject on oral aminosalicylates who has not been on stable doses for greater than, or discontinued within, at least 14 days prior to Baseline.

4. Subject taking oral corticosteroids:
Budesonide >9 mg/day
Beclomethasone >5 mg/day
Prednisone or equivalent >20 mg/day
Or has not been on the current course for [symbol] 14 days prior to Baseline and on a stable dose for [symbol] 7 days prior to Baseline

5. Subject on immunomodulators (AZA, 6-MP, MTX) who:
Has not been on the current course for [symbol] 42 days prior to Baseline, and [symbol]
Has not been on a stable dose for [symbol] 35 days prior to Baseline Medications and Treatments During the screening Period

6. Subject who received IV anti-infectives within 35 days prior to Baseline visit or oral/intramuscular anti-infectives (non-CD-related) within 14 days prior to the Baseline visit. This does not apply to TB prophylaxis.

7. Subject who received exclusive enteral nutrition or any parenteral nutrition within 35 days prior to Baseline.

8. Subject who received any live bacterial or viral vaccination within 30 days (8 weeks for Japan) prior to Screening or during the Screening Period.

9. Subject who received cyclosporine, tacrolimus, or mycophenolate mofetil within 35 days prior to Baseline.

10. Subject who received fecal microbial transplantation within 35 days prior to Baseline.

Prior Medications and Treatments

11. Subject who received any:
Approved biologics: infliximab, adalimumab, certolizumab, vedolizumab, natalizumab) within 8 weeks prior to Baseline, or ustekinumab within 12 weeks prior to Baseline

Note: If there was proper documentation of an undetectable drug level measured by a commercially available assay for any of the approved biologics above, there was no minimum washout prior to Baseline.
Any investigational biologic or other agent or procedure within 35 days or 5 half-lives prior to Baseline, whichever is longer.

12. Subject with prior exposure to p19 inhibitors (e.g., risankizumab).

13. Subject has been taking combination of two or more of the following: oral budesonide, or oral beclomethasone and/or oral prednisone (or equivalent) simultaneously, with the exception of inhalers, within 14 days prior to Screening or during the Screening period.

14. Subject who received IV/intramuscular corticosteroids within 14 days prior to Screening or during the Screening period.

15. Subject who received therapeutic enema or suppository, other than required for endoscopy, within 14 days prior to endoscopy used for Screening or during the Screening period.

16. Subject who received apheresis (e.g., Adacolumn apheresis) [symbol] 60 days prior to Screening or during the Screening period.

[symbol]

17. Subject who has concomitant cannabis use either recreational or for medical reasons within 14 days prior to Baseline or any history of clinically significant drug, or alcohol abuse in the last 12 months.

CD Related

18. Subject with currently known complications of CD such as:
abscess (abdominal or perianal),
symptomatic bowel strictures,
>2 missing segments of the following 5 segments: terminal ileum, right colon, transverse colon, sigmoid and left colon, and rectum
fulminant colitis,
toxic megacolon,
or any other manifestation that might require surgery while enrolled in the study.

19. Subject with ostomy or ileoanal pouch.

20. Subject diagnosed with short gut or short bowel syndrome.

21. Subject with surgical bowel resection within the past 3 months prior to Baseline, or a history of [symbol] 3 bowel resections.

Safety

22. Subject who has a known hypersensitivity to risankizumab or the excipients of any of the study drugs or the ingredients of Chinese hamster ovary (CHO).

23. Subjects with the following chronic or active infections:
Active, chronic, or recurrent infection that based on the Investigator's clinical assessment makes the subject unsuitable candidate for the study,
Infection with C. difficile toxin or other intestinal pathogen during Screening,
Are infected with human immunodeficiency virus (HIV),
QuantiFERON®-TB test or Purified Protein Derivative (PPD) skin test, or both, according to local guidelines, were performed during Screening. QuantiFERON®-TB test is preferred for subjects who received BCG vaccination or were exposed to other Mycobacteria species. Subjects with a positive test result (or indeterminate results that have been repeated) may participate in the study if further work up (according to local practice/guidelines) established conclusively that the subject has no evidence of active tuberculosis. Subjects with a history of active TB who had documented completion of a full course of anti-TB therapy could be allowed to enter the study after consultation with the AbbVie TA MD. If latent TB is established, TB prophylaxis/treatment should be initiated and maintained according to local country guidelines.
Have active hepatitis B or hepatitis C defined as:
   HBV: hepatitis B surface antigen (HBs Ag) positive (+), or detected sensitivity on the HBV deoxyribonucleic acid (DNA) polymerase chain reaction (PCR) qualitative test for hepatitis B core antibody (HBc Ab) positive subjects;
   HCV: HCV ribonucleic acid (RNA) detectable in any subject positive with anti-HCV antibody (HCV Ab)

24. Subject with a previous history of dysplasia of the gastrointestinal tract or found to have dysplasia, other than completely removed low-grade dysplastic lesions, in any biopsy performed during the Screening endoscopy.

25. Subject with a known history of lymphoproliferative disease, including lymphoma, or signs and symptoms suggestive of possible lymphoproliferative disease, such as lymphadenopathy and/or splenomegaly.

26. Subject with current or previous history of malignancy other than a successfully treated nonmetastatic cutaneous squamous cell or basal cell carcinoma or localized carcinoma in situ of the cervix.

27. Subject who has severe, progressive, or uncontrolled renal, hepatic, hematological, endocrine, disorder or symptoms thereof.

28. Female subjects who is pregnant, breastfeeding, or is considering becoming pregnant during the study or for approximately 140 days after the last dose of study drug.

29. Subject who has any condition, including any physical, psychological, or psychiatric condition, which in the opinion of the Investigator, would compromise the safety of the subject or the quality of the data and renders the subject an unsuitable candidate for the study.

30. Screening laboratory and other analyses show any of the following abnormal results:
Aspartate transaminase (AST), alanine transaminase (ALT) >2×upper limit of the reference range;
White blood cell (WBC) count <3.0×109/L;
Total bilirubin [symbol] 2 mg/dL; except for subjects with isolated elevation of indirect bilirubin relating to Gilbert syndrome;
Estimated glomerular filtration rate by simplified 4-variable Modification of Diet in Renal Disease (MDRD) formula <30 ml/min/1.73 m2.
Hemoglobin <8 g/dL
Platelets <100,000/μL
Positive serum pregnancy test at the Screening visit or positive urine pregnancy test at the Baseline visit.

Laboratory values could be re-tested once during the screening period after discussion and clearance with the TAMD. If the re-tested lab value(s) remained exclusionary, the subject was considered a screen failure. Redrawing samples if previous samples were unable to be analyzed would not count as a retest since previous result was never obtained.

31. No known active COVID-19 infection. Subject must not have signs/symptoms associated with COVID-19 infection.

Subjects who did not meet COVID-19 eligibility criteria were screen failed and could only rescreen after they met the following COVID-19 viral clearance criteria:
Symptomatic subjects: At least 2 negative viral tests in a row completed locally, [symbol] 24 hours apart after at least 10 days have passed since recovery, defined as resolution of fever without use of antipyretics and improvement in respiratory symptoms (e.g., cough, shortness of breath)
Asymptomatic subjects: At least 2 negative viral tests in a row completed locally, [symbol] 24 hours apart after at least 10 days have passed since prior positive result (Note: subjects who develop symptoms followed guidance above for symptomatic subjects)

Frequency or timing of COVID-19 testing and interval between testing for the above viral clearance criteria may be adjusted to account for epidemiological trends, updated information regarding infectivity and local/institutional guidelines.

Investigational Product:
Risankizumab

Doses:
Risankizumab 1200 mg IV Q4W*
Risankizumab 600 mg IV Q4W
Risankizumab 180 mg SC Q8W
Risankizumab 360 mg SC Q8W * If the 1200 mg dose was discontinued due to any reason, subjects continued to enroll into the study and be randomized to either 600 mg risankizumab or placebo at 2:1 ratio. The randomization ratio and sample size could be further updated in an amendment to the protocol. Subjects already randomized to treatment arms, including Induction Period 2, received blinded 600 mg risankizumab IV.

Mode of Administration:
Risankizumab solution for infusion (IV)
Risankizumab solution for injection (SC)

Reference Therapy: Placebo for risankizumab

Mode of Administration:
Placebo solution for infusion (IV)
Placebo solution for injection (SC)

Duration of Treatment: 12 or 24 weeks

The study included a Screening period of up to 35 days and a double-blind induction period of 12 weeks. All subjects who did not achieve clinical response at Week 12 were eligible to receive treatment in Induction Period 2 with risankizumab over a subsequent 12 week period. There was a follow up call 140 days from the last dose of study drug to obtain information on any new or ongoing AEs for those subjects who did not roll over into Study M16-000 or discontinue from the study prematurely.

Criteria for Evaluation:

Endpoint Definitions:
Clinical remission: average daily SF [symbol] 2.8 and not worse than Baseline AND average daily AP score [symbol] 1 and not worse than Baseline
Enhanced clinical response: [symbol] 60% decrease in average daily SF and/or [symbol] 35% decrease in average daily AP score and both not worse than Baseline, and/or clinical remission
Clinical response: [symbol] 30% decrease in average daily SF and/or [symbol] 30% decrease in average daily AP score and both not worse than Baseline
Endoscopic response: decrease in SES-CD >50% from Baseline (or for subjects with isolated ileal disease and a Baseline SES-CD of 4, at least a 2 point reduction from Baseline), as scored by central reviewer
Ulcer-free endoscopy: SES-CD ulcerated surface subscore of 0 in subjects with SES-CD ulcerated surface subscore [symbol] 1 at Baseline, as scored by a central reviewer
Endoscopic remission: SES-CD [symbol] 4 and at least a 2 point reduction versus baseline and no subscore greater than 1 in any individual variable, as scored by a central reviewer
CDAI clinical response: reduction of CDAI [symbol] 100 points from baseline
CDAI clinical remission: CDAI <150
SF remission: average daily SF [symbol] 2.8 and not worse than baseline
AP remission: average daily AP score [symbol] 1 and not worse than baseline

[symbol]

Efficacy: The US-specific protocol and the global protocol outside US have used different co-primary endpoints and multiplicity-controlled secondary endpoints.

For US-Specific Protocol:

Co-Primary Endpoints
The achievement of clinical remission (CDAI) at Week 12
The achievement of endoscopic response at Week 12

Secondary Endpoints
1. The achievement of clinical remission (SF/APS) at Week 12
2. The achievement of CDAI clinical response at Week 4
3. The achievement of CDAI clinical response at Week 12
4. Change from baseline in Functional Assessment of Chronic Illness Therapy-Fatigue (FACIT-Fatigue) at Week 12
5. The achievement of clinical remission (CDAI) at Week 4
6. The achievement of CDAI clinical response and endoscopic response at Week 12
7. The achievement of SF remission at Week 12
8. The achievement of AP remission at Week 12
9. The achievement of endoscopic remission at Week 12
10. The achievement of enhanced clinical response at Week 4
11. The achievement of ulcer-free endoscopy at Week 12
12. The achievement of enhanced clinical response at Week 12
13. The achievement of resolution of extra-intestinal manifestations (EIMs) at Week 12, in subjects with any EIMs at baseline
14. Occurrence of CD-related hospitalization through Week 12
15. The achievement of no draining fistulas at Week 12 in subjects with draining fistulas at baseline

For Global Protocol Outside US:

Co-Primary Endpoints
The achievement of clinical remission (SF/APS) at Week 12
The achievement of endoscopic response at Week 12

Secondary Endpoints
1. The achievement of clinical remission (CDAI) at Week 12
2. The achievement of CDAI clinical response at Week 4
3. The achievement of clinical remission (SF/APS) at Week 4
4. The achievement of CDAI clinical response at Week 12
5. Change from baseline in FACIT-Fatigue at Week 12
6. Change from baseline in Inflammatory Bowel Disease Questionnaire (IBDQ) total score at Week 12
7. The achievement of enhanced clinical response and endoscopic response at Week 12
8. The achievement of endoscopic remission at Week 12
9. The achievement of enhanced clinical response at Week 4
10. The achievement of ulcer-free endoscopy at Week 12
11. The achievement of enhanced clinical response at Week 12
12. The achievement of resolution of EIMs at Week 12, in subjects with any EIMs at baseline
13. Occurrence of CD-related hospitalization through Week 12
14. The achievement of no draining fistulas at Week 12 in subjects with draining fistulas at baseline
15. Change from baseline in Work Productivity and Impairment Questionnaire-Crohn's disease (WPAI-CD) Overall Work Impairment at Week 12
16. Change from baseline in Short Form-36 (SF-36) Physical Component Summary score at Week 12

Pharmacokinetics:

Serum risankizumab concentrations were determined from samples collected just prior to dosing at Weeks 4, 8, and 12/PD, and at Week 24 for subjects who received treatment in Induction Period 2. Additionally, intensive pharmacokinetic assessment was performed in 20 subjects after the 3rd induction dose (Week 8 to 12). For Subjects who consented to the intensive pharmacokinetic assessment, in addition to the time points above, blood samples were collected at Week 8 immediately after completion of infusion and 2 hours post completion of infusion, and at Weeks 9, 10, and 11.

Immunogenicity:

Serum ADAs were determined from samples collected just prior to dosing at Baseline and Weeks 4, 8, and 12/PD, and at Week 24 for subjects who received treatment in Induction Period 2.

Safety:

Safety analyses was performed on safety set which included all subjects who received at least one dose of study drug. Incidence of adverse events (AEs), changes in vital signs, physical examination results, and clinical laboratory data were assessed throughout the study.

Statistical Methods:

Efficacy:

The co-primary endpoints were the proportion of subjects with CDAI clinical remission at Week 12 and proportion of subjects with endoscopic response at Week 12. A total of approximately 855 subjects were randomized into two risankizumab treatment groups and the placebo group in a 2:2:1 ratio (342 subjects for risankizumab 600 mg dose group, 342 subjects for risankizumab 1200 mg dose group, and 171 subjects for placebo group). When all patients completed their Week 12/PD visit, the database was locked for the final analysis of 12-week induction period. When all the patients who entered the induction Period 2 finish Week 24/PD visit, the database was locked for the whole study and all the planned analysis for the induction Period 2 was performed.

Assuming the Week 12 CDAI clinical remission rate was 37% for one of the risankizumab dose groups and 17% for the placebo group, a sample size of 342 subjects for each of the risankizumab dose groups and 171 for the placebo group would have 99% power to detect the treatment difference between the risankizumab dose groups and placebo in CDAI clinical remission rates at Week 12 using Fisher's exact test at alpha of 0.025 (two-sided).

The bio-IR population was approximately 540 subjects. The study had 92% power to detect the treatment difference between one of the risankizumab dose groups and placebo in CDAI clinical remission at Week 12 using a Fisher's exact test a 0.025 significant level (two-sided) for the bio-IR population, assuming the Week 12 CDAI clinical remission rate was 34% for the risankizumab dose groups and 15% for the placebo group for bio-IR subjects. The non-bio-IR population was approximately 315 subjects. The study had 70% power to detect the treatment difference between one of the risankizumab dose groups and placebo in CDAI clinical remission, at Week 12 using a Fisher's exact test a 0.025 significant level (two-sided) for the non-bio-IR sub-population, assuming the Week 12 CDAI clinical remission rate was 42% for the risankizumab dose groups and 21% for the placebo group for non-bio-IR subjects.

The comparisons between each risankizumab dose versus placebo for the primary efficacy variable was performed using the Cochran-Mantel-Haenszel (CMH) test adjusted by prior biologic use (0, 1, >1) and Baseline steroid use (yes, no). Both of the co-primary efficacy endpoints were tested at statistically significant of 0.025 for each of the risankizumab dose groups versus placebo to adjust for multiplicity. A CMH based two-sided 95% confidence interval for the difference between treatment groups was calculated.

The intent-to-treat (ITT) set included all randomized subjects who had taken at least one dose of study drug. The primary population for efficacy analysis was the population of subjects in the intent-to-treat analysis set who had baseline eligibility SES-CD of [symbol] 6 ( [symbol] 4 for isolated ileal disease). Subjects who discontinued prior to Week 12 for any reason were considered as “not-achieved” for CDAI clinical remission and endoscopic response endpoints.

In general, continuous secondary efficacy variables were analyzed using a Mixed-Effect Model Repeated Measure (MMRM) model including factors for treatment group, visit, visit by treatment interaction, and stratification variables, for the longitudinal continuous endpoints. The MMRM analysis is considered primary for inferential purposes.

Pharmacokinetics and Immunogenicity:

Serum risankizumab concentrations were summarized at each time point for each dosing regimen using descriptive statistics. Population pharmacokinetic analyses combining the data from this study and other studies were performed. Relationships between risankizumab exposures and efficacy and safety variables of interest were explored.

ADA incidence was summarized by cohorts and study visits. ADA titers were tabulated for each subject at the respective study visits. The effect of ADAs on risankizumab pharmacokinetics, efficacy and/or safety variable(s) and/or any additional analyses were explored.

Safety:

Adverse events, laboratory data and vital signs were the primary safety parameters in this study. All safety comparisons were performed between treatment groups using the safety set. Treatment emergent AEs were defined as events that began or worsened either on or after the first dose of the study drug and within 140 days after the last dose of the study drug for subjects who did not participate in Study M16-000 or until first dose of study drug in Study M16-000 if the subject was enrolled in Study M16-000.

An overview of treatment-emergent AEs, AEs leading to death and AEs leading to premature discontinuation, AEs by Medical Dictionary for Drug Regulatory Activities (MedDRA version) preferred term and system organ class, AEs by maximum relationship to study drug, and AEs by maximum severity were summarized by number and percentage.

Changes in laboratory data were described using statistical characteristics and comparison between treatment groups was performed using a one-way Analysis of Variance (ANOVA). In addition, shift tables and listings were provided for abnormal values, whereby the normal range of the analyzing laboratory were used. Vital signs were analyzed similarly.

### Example 5: Protocol of the Phase 3 Maintenance Study M16-000

M16-00 was a multicenter, randomized, double-blind, placebo-controlled 52-week maintenance and an open-label extension study of the efficacy and safety of risankizumab in subjects with Crohn's disease (CD) who responded to induction treatment in M16-006 or M15-991; or completed M15-989.

Objectives:

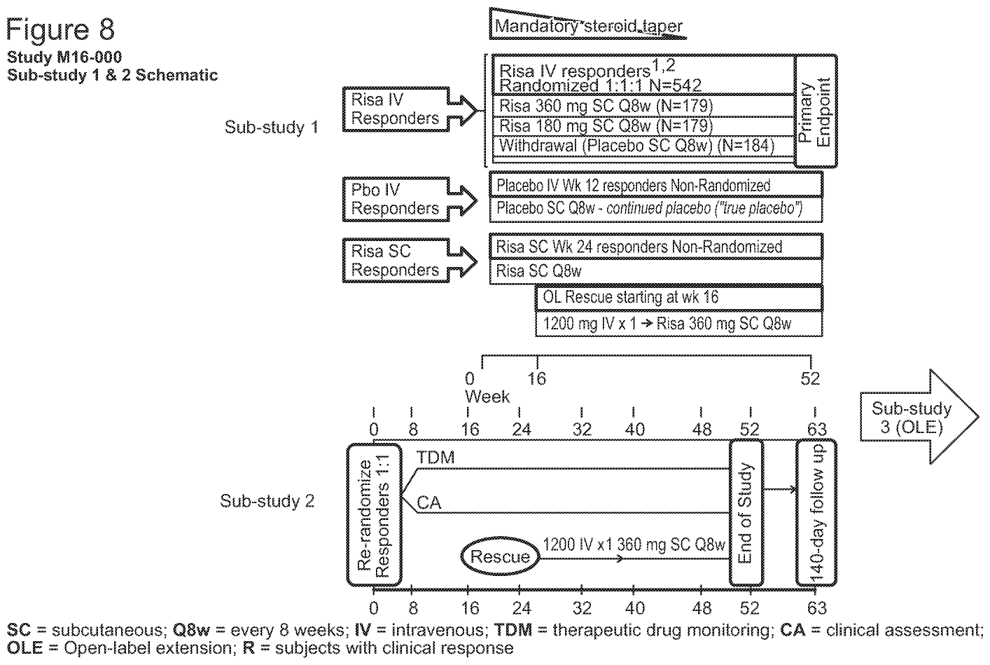

Sub-study 1: Study M16-000 sub-study 1 (SS1) was a Phase 3 multicenter and double-blind study that is a re-randomized responder withdrawal design to evaluate the efficacy and safety of risankizumab (RISA) as maintenance therapy versus withdrawal from risankizumab treatment (Withdrawal [placebo/PBO]) in subjects with moderately to severely active Crohn's disease who responded to 12 weeks of IV risankizumab induction treatment in Study M16-006 or Study M15-991 and had a Baseline (of induction) eligibility SES-CD of ≥6 (≥4 for isolated ileal disease) (FIG. 8). The study had different co-primary and secondary endpoints for US and ex-US.

Results of Sub-Study 1 of Study M16-000:

For the US-specific protocol, the study met co-primary endpoints demonstrating the superiority of both RISA 360 mg and RISA 180 mg subcutaneous (SC) dose compared to withdrawal (PBO).

The clinical remission per Crohn's disease activity index (CDAI) rate at Week 52 was statistically significantly higher in the RISA 360 mg and RISA 180 mg SC doses groups (51.4% and 54.8%, respectively) compared to the withdrawal (PBO) group (40.9%) with p-values=0.009 and 0.005 for 360 mg SC and 180 mg SC dose groups, respectively.

The endoscopic response rate at Week 52 was statistically significantly higher in the RISA 360 mg and RISA 180 mg SC doses groups (45.9% and 46.5%, respectively) compared to the withdrawal (PBO) group (22%) with p-value <0.001 for both dose groups.

For the global protocol outside of the US (ex-US), the sub-study met the co-primary endpoints demonstrating the superiority of RISA 360 mg group comparing to withdrawal (PBO) group. Numerically higher response rates were observed in RISA 180 mg group.

The clinical remission (per SF/APS: patient reported outcome of stool frequency and abdominal pain score which are components of CDAI) rate at Week 52 was statistically significantly higher in the RISA 360 mg SC dose group (51.1%) compared to the withdrawal (PBO) group (39.6%) with p-values=0.006; and was numerically higher in the RISA 180 mg SC dose group (45.9%) compared to the withdrawal (PBO) group (39.6%) with p-value=0.157.

The endoscopic response rate at Week 52 was statistically significantly higher in the RISA 360 mg SC dose group (45.9%) compared to the withdrawal (PBO) group (22%) with p-values <0.001; and was higher in the RISA 180 mg SC dose group (46.5%) compared to the withdrawal (PBO) group (22%) with p-value <0.001.

Sub-Study 2: Randomized, Exploratory Maintenance

To evaluate the efficacy and safety of two different dosing regimens for risankizumab (therapeutic drug monitoring vs clinical assessment for dose escalation) as maintenance therapy in subjects with moderately to severely active CD who responded to induction treatment in Study M16-006 or Study M15-991 (FIG. 8).

Sub-Study 3: Open-Label Long Term Extension

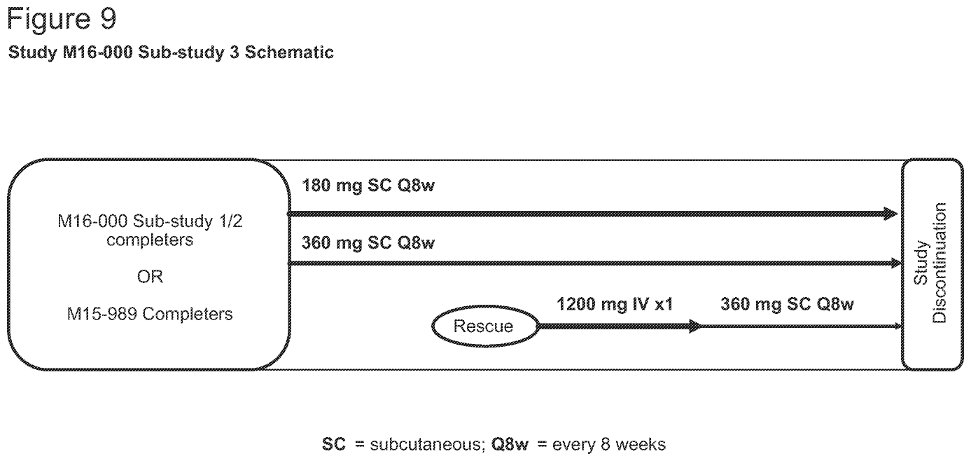

To evaluate long-term safety of risankizumab in subjects who completed Sub-study 1, Sub-study 2, the Phase 2, open-label extension study, Study M15-989 or subjects who responded to induction treatment in Study M16-006 or Study M15-991 with no final endoscopy due to the Covid-19 pandemic (FIG. 9). Additional objectives were to further investigate long-term efficacy and tolerability of risankizumab.

Investigators: Multicenter

Study Sites: Approximately 400 sites worldwide.

Study Population:

The study enrolled subjects who completed Study M16-006 or Study M15-991 and achieved clinical response, defined as [symbol] 30% decrease in average daily stool frequency (SF) and/or [symbol] 30% decrease in average daily abdominal pain (AP) score and both not worse than Baseline of the induction study. When calculating SF, only the number of liquid or very soft stools were recorded. The Sub-study 3 could also enroll subjects who completed Study M15-989 and subjects who responded to induction treatment in Study M16-006 or Study M15-991 with no final endoscopy due to the Covid-19 pandemic.

Main Entry Criteria for Study M16-006 and Study M15-991:
Males and females aged [symbol] 18 to [symbol] 80 years of age, or minimum age of adult consent according to local regulations, at the Baseline Visit. Where locally permissible [symbol] 16 to <18 years of age who meet the definition of Tanner stage 5 development.
Moderately to severely active CD, defined as average daily SF [symbol] 4 and/or average daily AP score [symbol] 2.
Endoscopic evidence of mucosal inflammation (Simple Endoscopic Score for CD [SES-CD], excluding the presence of narrowing component, [symbol] 3 as confirmed by a centrally read endoscopy. During the Covid-19 pandemic the final endoscopy post-induction treatment may not be conducted due to local regulation prohibiting endoscopy and subjects may be allowed to enroll in Sub-study 3 should they meet clinical response.

Main Entry Criteria for Study M15-989:

Patients with Crohn's disease, who successfully completed the preceding Trial 1311.6 (Study M15-993). Successful treatment was defined as:
Completion of Period 2 in Study 1311.6 (M15-993) with a clinical response (drop in CDAI from baseline by [symbol] 100) but no remission (CDAI <150) at Visit E1; or
Completion of Period 3 in Study 1311.6 (M15-993) with a clinical response (drop in CDAI from baseline by [symbol] 100) or remission (CDAI <150) at Visit E5.

Number of Subjects to be Enrolled:

Approximately 1250 (final subject number is determined by the clinical response rate of the induction studies).

Sub-study 1 enrolled first. Subjects who achieved clinical response to study drug after induction continued to enroll until approximately 450 subjects who achieved clinical response to IV risankizumab and a Baseline of induction eligibility SES-CD of [symbol] 6 (or [symbol] 4 for isolated ileal disease) were randomized. The sponsor could enroll up to 15% more subjects over the current planned sample size to accommodate the potential missed visits due to COVID-19 pandemic impact.

Sub-study 2 enrolled the remaining subjects who achieved clinical response to study drug. Subjects who completed Sub-studies 1, 2 or Study M15-989 were eligible for Sub-study 3. In addition, subjects who had final endoscopy for Studies M16-006 or M15-991 missed due to local regulation prohibiting endoscopy during the Covid-19 pandemic were allowed to enroll directly in Sub-study 3 if they met clinical response.

Methodology:

This was a Phase 3, multicenter study that consisted of three sub-studies: Sub-study 1 was a 52 week randomized, double-blind, placebo-controlled maintenance study. Sub-study 2 was a 52 week randomized, exploratory maintenance study. Sub-study 3 was an open-label (OL) long-term extension for subjects who completed Sub-study 1, 2, Study M15-989, or subjects who responded to induction treatment in Study M16-006 or Study M15-991 with no final endoscopy due to the Covid-19 pandemic.

For subjects who enrolled in Sub-study 1 or Sub-study 2, Baseline was defined as the Baseline Visit of the induction Study M16-006 or Study M15-991, and Week 0 was defined as the first study visit in Sub-study 1 or Sub-study 2. The final visit of Study M16-006 or Study M15-991 (Week 12 or Week 24) was considered as the Week 0 visit of Study M16-000 Sub-studies 1 and 2. The duration of Sub-studies 1 and 2 was up to 68 weeks, including a 52-week maintenance period and a 140-day follow-up period (except for subjects who continue in Sub-study 3) from last dose of study drug.

For subjects who enrolled in Sub-study 3 directly from Study M15-989, Baseline was defined as the Baseline Visit in Study M15-993 (the preceding Phase 2 study). These subjects began Sub-study 3 at Week 56 as their first visit.

Sub-study 3 lasted up to 220 weeks, or until the study was discontinued, whichever was earlier. Details of missing visits, partial visits, or virtual visits due to COVID-19 were collected.

Sub-Study 1 (Randomized, Double-Blind, Placebo-Controlled Maintenance) Results

Study M16-00 sub-study 1 (SS1) was a Phase 3 multicenter and double-blind study that was a re-randomized responder withdrawal design. The objective was to evaluate the efficacy and safety of RISA versus withdrawal from RISA as maintenance therapy in subjects with moderately to severely active CD who responded to 12-weeks of IV risankizumab induction treatment in Study M16-006 or Study M15-991. Sub-study 1 had two portions: randomized portion and non-randomized portion, as shown in FIG. 8 (top).

Demographic, Baseline Characteristics, and Subject Disposition:

A total of 712 subjects were enrolled in sub-study 1, where 542 were in the randomized portion and 170 were in the non-randomized portion. Among the 542 randomized subjects, 489 had baseline eligible SES-CD of [symbol] 6 ( [symbol] 4 for isolated ileal disease), and 53 subjects had a lower baseline eligible SES-CD who were not included in the primary analyses. All randomized subjects received at least one dose of study drug.

Of the 489 subjects with a baseline eligible SES-CD [symbol] 6 ( [symbol] 4 for isolated ileal disease):
462 received 12 weeks of IV risankizumab induction, and represent the primary intent-to-treat population for efficacy analysis (ITT1A)(141 in the RISA 360 mg SC group, 157 in the RISA 180 mg SC group and 164 in the PBO group)
23 subjects received 24-weeks of IV risankizumab induction,
4 subjects were excluded from efficacy from a site with significant non-compliance.

Key demographics and baseline characteristics were generally balanced between three treatment groups. Discontinuation rates were similar among treatment groups. The full study schematic for all sub-studies in M16-000 is presented in FIGS. 8 and 9.

The Randomized Portion:

Approximately 450 subjects who achieved clinical response to IV risankizumab at the end of Study M16-006 or Study M15-991 and a Baseline of induction eligibility SES-CD of [symbol] 6 (or [symbol] 4 for isolated ileal disease) were re-randomized in a 1:1:1 ratio to one of the following three treatment groups for 52 weeks (the sponsor could enroll up to 15% more subjects over the current planned sample size to accommodate the potential missed visits due to COVID-19 pandemic impact):

Group 1: Risankizumab 180 mg subcutaneous (SC) Q8w (n=150)

Group 2: Risankizumab 360 mg SC Q8w (n=150)

Group 3: withdrawal (PBO) (n=150)

Subjects who achieved clinical response to study drug after induction and had a Baseline eligibility SES-CD of <6 (<4 for isolated ileal disease) were re-randomized in the same way but are analyzed separately for exploratory purposes.

Only subjects who achieved clinical response after IV risankizumab at Week 12 of the induction study or Week 24 of Induction Period 2 in the induction study were randomized to Group 1, 2, or 3 in the Substudy 1 of the Study M16-000. Subjects achieving clinical response after SC risankizumab at Week 24 of Induction Period 2 in the induction study were eligible to enroll in Sub-study 1, but were assigned by Interactive Response Technology (IRT) to receive blinded risankizumab 180 mg or 360 mg SC at the same dose they received during Induction Period 2 of the induction study Q8w and were excluded from the primary analysis.

The primary efficacy population consisted of randomized subjects who had baseline eligible SES-CD of [symbol] 6 ( [symbol] 4 for isolated ileal disease) and received 12 weeks of IV risankizumab as induction therapy in either M16-006 or M15-991. Subjects with a lower baseline SES-CD and subjects who received 24 weeks of IV risankizumab during induction were randomized but excluded from the primary population for efficacy analysis per FDA recommendation. The randomization stratification factors were endoscopic response (yes or no, per local read) and clinical remission status (yes or no) at the last visit of the induction study, as well as by last IV dose during risankizumab induction periods (1200 mg or 600 mg).

Subjects with clinical response to placebo in Study M16-006 or Study M15-991 were assigned by IRT to continue to receive blinded placebo and are excluded from the primary efficacy analysis.

Subjects were stratified based on endoscopic response (per local read) and clinical remission status from the last visit of Study M16-006 or Study M15-991, as well as by risankizumab induction dose. During the Covid-19 pandemic the final endoscopy could be missing for Studies M16-006 or M15-991 due to local regulation prohibiting endoscopy and subjects could be allowed to enroll in Sub-study 3 if they meet clinical response.

Subjects who demonstrated inadequate response (IR) during Sub-study 1 could receive OL risankizumab rescue therapy starting at the Week 16 Visit based upon increased symptom activity and confirmation with objective markers of inflammation.

The Non-Randomized Portion:

Subjects achieving clinical response after either RISA 180 mg SC or RISA 360 mg SC at Week 24 (Induction Period 2) in the induction studies continued their Induction Period 2 dose. Subjects with clinical response to IV placebo at Week 12 in the induction study continued to receive placebo.

All subjects in both randomized portion and non-randomized portion were included in the safety analysis.

Sub-Study 2 (Randomized, Exploratory Maintenance):

Enrollment for Sub-study 2 initiated after completion of enrollment for Sub-study 1. Subjects who achieved clinical response at the end of Study M16-006 or Study M15-991, regardless of induction or Induction Period 2 treatment, were randomized 1:1 into an exploratory 52 week maintenance study for comparison between two treatment regimens: clinical assessment (CA) for dose escalation and therapeutic drug monitoring (TDM) for dose escalation (treating to target levels of isankizumab).

In order to ensure subjects approached steady state for risankizumab by Week 16 and to maintain the double blind from induction, treatment at the Week 0 Visit was blinded:
Subjects with clinical response to risankizumab received isankizumab 180 mg SC and placebo intravenous (IV).
Subjects with clinical response to placebo received placebo SC and risankizumab 1200 mg IV.

All subjects received OL isankizumab 180 mg SC starting at Week 8. Risankizumab rescue therapy could be administered at any time on or after the Week 16 visit and was allowed as per IR criteria described in the “Risankizumab Rescue Therapy” protocol. Subjects were evaluated at each scheduled visit for risankizumab rescue therapy.

Subjects in the CA and TDM arms received risankizumab rescue therapy based on the criteria in Table 9.

Results of serum risankizumab analysis were provided to the site in approximately 2 weeks. All risankizumab samples were shipped the same day they were collected at Weeks 16, 24, 32, 40, and 48 to ensure adequate processing time to determine serum concentration levels needed for dosing adjustments.

Sub-Study 1 and 2

Subjects used the same subject diary that was dispensed for Studies M16-006 or M15-991. Subjects were also dispensed a patient information card for Study M16-000. Visits occurred at Weeks 0, 8, 16, 24, 32, 40, 48, and 52/Premature Discontinuation (PD) to collect clinical and laboratory assessments of disease activity. Additionally, subjects completed symptom, quality of life (QoL) and work productivity questionnaires throughout the study. Clinical labs including, but not limited to, urinalysis, chemistry and hematology, high-sensitivity C-reactive protein (hs-CRP), serum isankizumab concentrations, and serum anti-drug antibody (ADA) levels were collected at the visits indicated. In addition, stool samples for calprotectin (FCP) analysis were collected and be taken before starting bowel preparations for endoscopy. An endoscopy occurred at the Week 52/PD visit and was evaluated using SES-CD, confirmed by a central reader. Biopsy to rule out dysplasia/malignancy was performed at the same time as the endoscopy. Exploratory research samples were taken during Sub-study 1. Subjects who discontinued from the study early or completed Sub-study 1 or 2 and did not continue into Sub-study 3 had an additional 140 days of safety follow-up from the last dose administration of study drug.

Sub-Study 3: OL Extension

Subjects who completed Sub-study 1, 2 or Study M15-989 could enter Sub-study 3, an OL extension study. All study activities, including endoscopy must have been completed for a subject to enter Sub-study 3. Subjects received OL risankizumab based on their assignment during Sub-study 1, 2 or Study M15-989:
Subjects who completed Sub-study 1 or 2 without receiving risankizumab rescue therapy: risankizumab 180 mg SC Q8w.
Subjects who completed Sub-study 1 or 2 and received risankizumab rescue therapy: risankizumab 360 mg SC Q8w.
Subjects who completed Study M15-989 continued to receive risankizumab 180 mg SC Q8w treatment and were eligible to receive rescue therapy as described below.
Subjects who completed Studies M16-006 or M15-991 without endoscopy performed and enrolled directly into Sub-study 3 due to Covid-19 pandemic received risankizumab open label 180 mg SC Q8w.

All subjects who continued in Sub-study 3 had OL risankizumab syringes dispensed at Week 56 at an onsite visit. At the Week 56 Visit, subjects who completed Sub-study 1, Sub-study 2 or subjects enrolled directly from Study M16-006 or Study M15-991 were monitored for 1 hour after SC administration of drug. This ensured that subjects who received placebo during induction and Substudy 1 and received risankizumab SC for the first time were monitored for any injection related events. Subjects who completed Study M15-989 were monitored by site until judged clinically stable. Additional Visits occurred every 24 weeks (where locally permitted) starting at Week 56 and enough risankizumab syringes were dispensed for at home injections (if allowed per local requirements) in between all visits. Endoscopies occurred every 96 weeks starting at Week 152. The same subject diary could be used as during Sub-study 1, Sub-study 2 or the induction Studies M16-006 and M15-991. Subjects enrolled from Study M15-989 were dispensed a subject diary.

Sub-Study 3: OL Extension (Continued)

Subjects who demonstrated IR during Sub-study 3 could receive OL risankizumab rescue therapy as described in the “Risankizumab Rescue Therapy” section. Doses of CD-related antibiotics, aminosalicylates, corticosteroids or immunomodulators could be changed at the discretion of the Investigator and needed to be documented on the appropriate eCRF.

Open-Label Risankizumab Rescue Therapy:

Definition of Inadequate Response (IR)

Subjects in Sub-study 1, the CA arm of Sub-study 2, and Sub-study 3 received risankizumab rescue therapy based upon:

1. clinical symptoms: average daily SF [symbol] 3.3 and/or average daily AP score [symbol] 1.5 confirmed by

2. an objective marker of inflammation
1. hs-CRP [symbol] 5 mg/L and/or FCP [symbol] 250 μg/g, or if hs-CRP and/or FCP are not elevated,
2. SES-CD, excluding the narrowing component, [symbol] 6 ( [symbol] 4 for isolated ileal disease), as scored by the site Investigator* *Note: At any scheduled Study Visit with endoscopy (e.g., Week 52 for Sub-studies 1 or 2, and Week 152 of Sub-study 3), IR could be defined by clinical symptoms and SES-CD criteria above. If subject did not meet SES-CD criteria for IR, hs-CRP and fecal calprotectin could be used. An Investigator could also perform an endoscopy at their own discretion and IR could be defined by the SES-CD criteria above. Subjects in the TDM arm of Sub-study 2 received risankizumab rescue therapy based upon the criteria listed above for clinical symptoms and objective markers for inflammation, as well as serum risankizumab levels as indicated in Table 9.

Subjects in Sub-study 2 were evaluated at each Study Visit for IR. For subjects in the TDM arm, the serum risankizumab level from the prior study visit was used for evaluation, except at the Week 16 Study Visit where the dose escalation algorithm used the serum risankizumab level from the same visit*. *Note: Once Week 16 serum risankizumab levels were available, sites were notified if the subject needed to return to clinic to receive rescue therapy.

TABLE 9
IR Criteria to Receive Risankizumab Rescue Therapy

| | | | |
|---|---|---|---|
| | | | Risankizumab |
| [RZB] Serum | Symptom | Objective Markers of | Rescue |
| (μg/mL) | Activity¹ | Inflammation^{2, 3} | Therapy? |

Sub-study 1, CA Arm Sub-study 2, Sub-study 3

| | | | |
|---|---|---|---|
| N/a | Low | N/a | No |
| N/a | High | Low | No |
| N/a | High | High | Yes |

TDM Arm Sub-study 2

| | | | |
|---|---|---|---|
| <4 | Any | Any | Yes |
| ≥4 and <8 | Low | N/a | No |
| ≥4 and <8 | High | Low | No |
| ≥4 and <8 | High | High | Yes |
| ≥8 | Any | Any | No⁴ |

¹Average daily SF ≥3.3 and/or average daily AP score ≥1.5.
²High levels: hs-CRP ≥5 mg/L and/or FCP ≥250 μg/g.
³If hs-CRP nor FCP are elevated, an endoscopy confirming active disease could also be confirmatory. An SES-CD, excluding the narrowing component, of ≥6 (or ≥4 for isolated ileal disease) is high activity.
⁴Subjects who have high symptom activity, high objective markers of inflammation, and high serum levels of risankizumab could be withdrawn from the study if they had been on a stable dose of risankizumab for >8 weeks.

Risankizumab Rescue Therapy Dose and Timing of Visits

If a subject had IR as defined above, they could receive risankizumab rescue therapy, which is one dose of 1200 mg IV followed by 360 mg SC through the end of the trial. Rescue Visit was defined as the date on which the subject was administered 1200 mg IV. All attempts were made to keep subjects on their original SC dosing regimen of Q8w intervals. This aligned with scheduled Study Visits in Sub-study 1 and 2. In Sub-study 3, every third dose (Q8w) corresponded with an on-site visit (every 24 weeks). The study medication schedule when the subjects moved to risankizumab rescue depended on when the Rescue Visit occurred:
If the Rescue Visit occurred within the 7 days prior to a scheduled study visit or on the same day of the scheduled visit, both visits (rescue and scheduled) were combined and the subject only received 1200 mg risankizumab IV at the site.
If the Rescue Visit occurred after the planned scheduled visit, within the 7 day window, and the subject did not received the SC dose yet, both visits (rescue and scheduled) were also combined and the subject only received 1200 mg risankizumab IV at the site.
If the Rescue Visit occurred after the scheduled study visit and the subject already received the SC dose at the scheduled visit, the subject received 1200 mg risankizumab IV at the Rescue Visit.

Risankizumab Rescue Therapy Dose and Timing of Visits (Continued)

After the IV dose, the subject received 360 mg risankizumab SC at the next scheduled dose (may be less than 8 weeks) through the end of the study.

During Sub-study 1 and 2, the evaluation for risankizumab rescue therapy could begin at the Week 16 Visit. Subjects could have up to two Rescue Visits during Sub-study 1 or 2, but must meet the definition of IR as described above for each Rescue Visit and the Rescue Visits must be [symbol] 16 weeks apart.

During Sub-study 3, subjects could receive up to 2 Rescue Visits per year and the Rescue Visits must be [symbol] 16 weeks apart. Subjects could receive up to 3 Rescue Visits in total during Sub-study 3.

Diagnosis and Main Criteria for Inclusion/Exclusion:

Main Inclusion:

1. Entry and completion of Study M16-006, Study M15-991 or Study M15-989. Completion included the final endoscopy of Study M16-006, Study M15-991, or Study M15-989. The final endoscopy for Studies M16-006 or M15-991 could be missing during the Covid-19 pandemic due to local regulation prohibiting endoscopy and subjects could be allowed to enroll in Sub-study 3 should they meet clinical response.

2. Achieved clinical response, defined as [symbol] 30% decrease in average daily SF and/or [symbol] 30% decrease in average daily AP score, and both not worse than Baseline of the induction study, at t ie last visit of Study M16-006 or Study M15-991. This was not applicable for subjects enrolling from Study M15-989.

3. If female, subject must have been either postmenopausal or permanently surgically sterile, or for women of childbearing potential, continue practicing birth control through at least 140 days after the last dose of study drug.

4. Subject must have been able and willing to give written informed consent and comply with the requirements of this study protocol, including self-administration or care-giver administration of SC injections. In Japan, if the subject is <20 years old, a subject's parent or legal guardian must have been willing to give written informed consent.

Main Exclusion:

1. Subject was considered by the Investigator, for any reason, to be an unsuitable candidate for the study. Subjects should not be enrolled in Study M16-000 with high grade colonic dysplasia or colon cancer identified during Study M15-991, Study M16-006 or during Study M15-989 if the final endoscopy was performed prior to enter Study M16-000.

2. Subject who has a known hypersensitivity to risankizumab or the excipients of any of the study drugs or the ingredients of CHO, or had an AE during Studies M16-006, M15-991 or M15-989 that in the Investigator's judgment makes the subject unsuitable for this study.

3. Confirmed positive urine pregnancy test at the Final Visit of Study M16-006, Study M15-991 or Study M15-989.

4. Subject was not in compliance with prior and concomitant medication requirements throughout Studies M16-006, M15-991 or M15-989.

5. Subject with any active or chronic recurring infections based on the Investigator's assessment made the subject an unsuitable candidate for the study.

6. Have a known history of lymphoproliferative disease, including lymphoma, or signs and symptoms suggestive of possible lymphoproliferative disease, such as lymphadenopathy and/or splenomegaly.

Investigational Product: Risankizumab

Doses:
Risankizumab 1200 mg IV*
Risankizumab 360 mg SC Q8w
Risankizumab 180 mg SC Q8w *If administration of the 1200 mg IV dose was halted for any reason, it was replaced with a 600 mg IV.

Mode of Administration:
Risankizumab solution for infusion (IV)
Risankizumab solution for injection (SC)

Reference Therapy: Placebo for risankizumab

Doses: Placebo: N/A

Mode of Administration:
Placebo solution for infusion (IV)
Placebo solution for injection (SC)

Duration of Treatment: Sub-study 1 and 2 had a duration of 52 weeks and Sub-study 3 lasted for up to 220 weeks, or until the study was discontinued, whichever was earlier.

Subjects who prematurely discontinued the study have a 140-day follow-up phone call for safety purposes from the last administration of study drug.

Endpoint Definitions: The following definitions applied to the efficacy variables described below:
Clinical remission (SF/APS): average daily stool frequency (SF) [symbol] 2.8 and not worse than Baseline of the induction study AND average daily AP score [symbol] 1 and not worse than Baseline of the induction study.
Enhanced clinical response: [symbol] 60% decrease in average daily SF and/or [symbol] 35% decrease in average daily AP score and both not worse than Baseline of the induction study, and/or clinical remission
Clinical response: [symbol] 130% decrease in average daily SF and/or [symbol] 30% decrease in average daily AP score and both not worse than Baseline of the induction study
Endoscopic response: decrease in SES-CD >50% from Baseline of the induction study (or for subjects with isolated ileal disease and an SES-CD of 4 at Baseline of the induction study, at least a 2 point reduction from Baseline of the induction study), as scored by central reviewer
Maintenance of clinical remission (CDAI or SF/APS): Clinical remission at Week 52 in subjects who achieved clinical remission at Week 0
Steroid-Free for 90 days clinical remission (CDAI or SF/APS): Discontinuation of corticosteroid use for 90 days and clinical remission at Week 52 in subjects taking steroids at baseline of the induction study
Ulcer-free endoscopy: SES-CD ulcerated surface subscore of 0 in subjects with SES-CD ulcerated surface subscore [symbol] 1 at Baseline of the induction study, as scored by a central reviewer
Endoscopic remission: SES-CD [symbol] 4 and at least a 2 point reduction versus baseline and no subscore greater than 1 in any individual variable, as scored by a central reviewer
Deep remission (CDAI or SF/APS): clinical remission (CDAI or SF/APS) and endoscopic remission
CDAI clinical response: reduction of CDAI [symbol] 100 points from baseline of the induction study
SF remission: Average daily SF [symbol] 2.8 and not worse than baseline
AP remission: Average daily AP score [symbol] 1 and not worse than baseline
Clinical remission (CDAI): CDAI <150

[symbol]

Criteria for Evaluation:

Sub-Study 1 (Risankizumab Vs Placebo)

For US-Specific Protocol:

Co-Primary Endpoint: The achievement of clinical remission (per CDAI) at Week 52 and the achievement of endoscopic response at Week 52.

Secondary Endpoints:

1. The achievement of clinical remission (per SF/APS) at Week 52

2. The achievement of clinical remission (per CDAI) at Week 52 among the subjects with CDAI clinical remission at Week 0

3. The achievement of ulcer-free endoscopy at Week 52

4. The achievement of endoscopic remission at Week 52

5. Change from baseline of the induction study in Functional Assessment of Chronic Illness Therapy-Fatigue (FACIT-Fatigue) at Week 52

6. The achievement of discontinuation of corticosteroid use for 90 days and clinical remission (per CDAI) at Week 52 in subjects taking steroids at baseline of the induction study

7. The achievement of CDAI clinical response at Week 52

8. The achievement of SF remission at Week 52

9. The achievement of AP remission at Week 52

10. The achievement of clinical remission (per CDAI) and endoscopic response at Week 52

11. The achievement of deep remission (per CDAI) at Week 52

12. Exposure adjusted occurrence of CD-related hospitalizations from Week 0 through Week 52

For the Global Protocol Outside of the US:

Co-Primary Endpoint: The achievement of clinical remission (per SF/APS) at Week 52 and the achievement of endoscopic response at Week 52

Secondary Endpoints:

1. The achievement of clinical remission (per CDAI) at Week 52

2. The achievement of clinical remission (per SF/APS) at Week 52 among the subjects with clinical remission at Week 0

3. The achievement of ulcer-free endoscopy at Week 52

4. The achievement of endoscopic remission at Week 52

5. Change from baseline of the induction study in Inflammatory Bowel Disease Questionnaire (IBDQ) total score at Week 52

6. Change from baseline of the induction study in FACIT-Fatigue at Week 52

7. The achievement of discontinuation of corticosteroid use for 90 days and clinical remission (per SF/APS) at Week 52 in subjects taking steroids at baseline of the induction study

8. The achievement of CDAI clinical response at Week 52

9. The achievement of clinical remission (per SF/APS) and endoscopic response at Week 52

10. The achievement of enhanced clinical response at Week 52

11. The achievement of deep remission (per SF/APS) at Week 52

12. Exposure adjusted occurrence of CD-related hospitalizations from Week 0 through Week 52

13. Change from baseline of the induction study in Short Form-36 (SF-36) Physical Component Summary score at Week 52

12. Exposure adjusted occurrence of CD-related hospitalizations from Week 0 through Week 52

The overall type I error rate for the co-primary and multiplicity-controlled secondary endpoints for the two dose groups was strongly controlled at the a level of 0.05 (2-sided) using a graphical approach. Statistical testing was performed for the two co-primary endpoints in RISA 360 mg group vs. the placebo group, followed by the two co-primary endpoints in RISA 180 mg group vs. the placebo group before proceeding to test secondary endpoints.

Pharmacokinetics:

Serum risankizumab concentrations were determined from samples collected just prior to dosing at Week 0, Week 16, Week 32, Week 48, Week 52 and/or PD during Sub-studies 1 and 2 and every 24 weeks/PD during Sub-study 3. Samples were also collected at the time of initiation of risankizumab rescue therapy and unscheduled visits. Additionally, for subjects in Sub-study 2 samples were also collected at Week 8, Week 24 and Week 40.

Immunogenicity:

Serum ADA was determined from samples collected just prior to dosing at Week 0, Week 16, Week 32, Week 48, Week 52 and/or PD during Sub-studies 1 and 2, Week 56 and every 24 weeks/PD during Sub-study 3. Samples were also collected at the time of initiation of risankizumab rescue therapy and unscheduled visits. Additionally, for subjects in Sub-study 2 samples were also collected at Week 8, Week 24 and Week 40.

Safety:

Safety analyses were performed on safety set which included all subjects who received at least one dose of study drug. Incidence of adverse events (AEs), changes in vital signs, physical examination results, and clinical laboratory data were assessed throughout the study.

Safety data was prepared for all subjects who received at least one dose of study drug in M16-000 sub-study 1. The RISA 52-Week maintenance treatment with both 180 mg and 360 mg SC doses was generally safe and well-tolerated. No new safety risks were identified, and the overall safety profile was consistent with the known safety profile of RISA. The RISA 52-Week maintenance treatment with 180 mg SC and 360 mg SC was generally safe and well-tolerated. The overall safety profile was consistent with the known safety profile of RISA. No new safety risks were identified.

Statistical Methods:

Efficacy:

The co-primary endpoints were the proportion of subjects with clinical remission at Week 52 and the proportion of subjects who achieved endoscopic response at Week 52. A total of approximately 450 subjects were expected to be randomized into 2 risankizumab treatment groups and the placebo group in a 1:1:1 ratio (150 subjects for risankizumab 180 mg SC Q8w dose group, 150 subjects for risankizumab 360 mg SC Q8w dose group, and 150 subjects for placebo group). The sponsor could enroll up to 15% more subjects over the current planned sample size to accommodate the potential missed visits due to COVID-19 pandemic impact.

Sample size calculation was based on the larger sample size needed to detect treatment difference for each of the co-primary endpoints. Since historical data showed slightly lower event rate and similar treatment difference versus placebo for the endoscopic response rate than the clinical remission rate at Week 52, clinical remission rates at Week 52 were used for power calculation. Assuming the Week 52 clinical remission rate is 38.7% for one of the risankizumab dose groups and 20% for the placebo group, a sample size of 150 subjects for each of the risankizumab dose group and 150 for the placebo group has approximately 93% power to detect the treatment difference between one of the risankizumab dose groups and placebo in clinical remission rates at Week 52 using a Fisher's exact test at alpha level of 0.05 (two-sided).

The comparisons between each of the risankizumab dose group versus placebo for the primary efficacy variable was performed using the Cochran-Mantel-Haenszel (CMH) test adjusted by Study M16-000 Week 0 clinical remission status and Study M16-000 Week 0 endoscopic response (Week 12 or 24 of Study M16-006 or Study M15-991) status (per central read), and risankizumab induction dose. A multiple testing procedure was used to provide strong control of the type I error rate at alpha=0.05 (2-sided) across analyses comparing each risankizumab dose group to placebo with respect to the coprimary endpoints, and ranked secondary endpoints. A CMH based two-sided 95% confidence interval for the difference between treatment groups was calculated.

The intent-to-treat (ITT) analysis set included all subjects who were randomized and received at least one dose of study drug.
Sub-study 1: The intent-to-treat analysis set for Sub-study 1 (denoted as ITT1) was the randomized subjects who received at least one dose of study drug in the Sub-study 1. In addition, the intent-to-treat analysis set ITT1A included the subjects in ITT1 set who had Baseline eligibility SES-CD of [symbol] 6 ( [symbol] 4 for isolated ileal disease). ITT1A was the primary analysis set for efficacy analysis of Sub-study 1.
Sub-study 2: The intent-to-treat analysis set for Sub-study 2 (denoted as ITT2) was the randomized subjects who received at least one dose of study drug in Sub-study 2 of the study.
Sub-study 3: The intent-to-treat analysis set for Sub-study 3 (denoted as ITT3) included all subjects who received at least one dose of study drug in the Sub-study 3 of the study.

If average daily SF or AP score at Week 52 was missing, the non-responder imputation (NRI) approach was applied. Subjects who discontinued prior to Week 52 for any reason or switch to OL risankizumab were considered as “not-achieved” for clinical remission and endoscopic response endpoints.

In general, continuous secondary efficacy variables were analyzed using a Mixed-Effect Model Repeated Measure (MMRM) model including factors for treatment group, visit, visit by treatment interaction, and stratification variables, for the longitudinal continuous endpoints. The MMRM analysis was considered primary for inferential purposes.

For the US-specific protocol, sub-study 1 met the co-primary endpoints for both RISA 360 mg and 180 mg SC dose groups compared to the withdrawal (PBO) group (p-values=0.009 and 0.005 for 360 mg and 180 mg, respectively for clinical remission (CDAI) and all p-values <0.001 for endoscopic response). The results for multiplicity-controlled secondary endpoints were presented in Table 10(A1) and Table 10(A2).

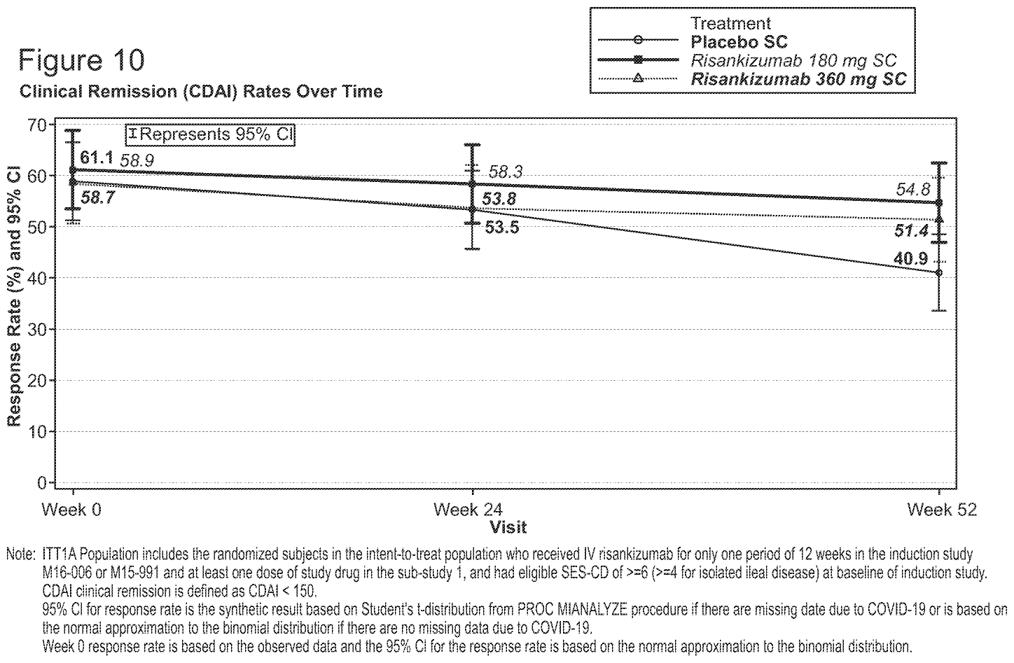

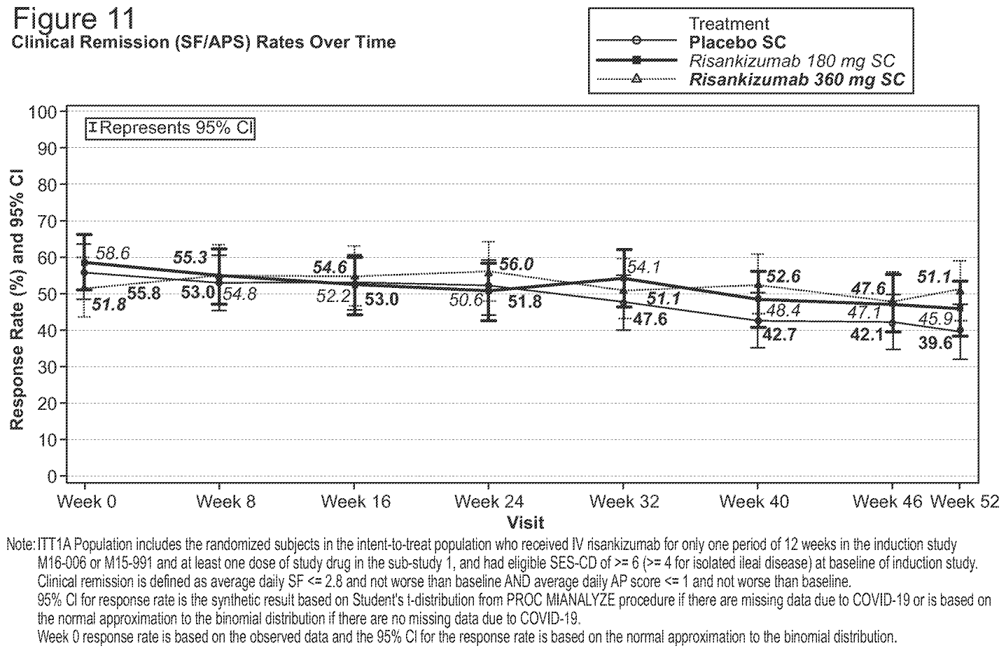

For the global protocol outside US, sub-study 1 met the co-primary endpoints for RISA 360 mg group compared to the withdrawal (PBO)group (p-value=0.006 for clinical remission (SF/APS) and p-value <0.001 for endoscopic response). However, the study did not meet the co-primary endpoints for 180 mg SC dose group compared to the withdrawal (PBO)group (p-value=0.157 for clinical remission (SF/APS) and p-value <0.001 for endoscopic response). The results for multiplicity-controlled secondary endpoints were presented in Table 10(B1) and Table 10(B2). Clinical remission rates over time are shown in FIGS. 10 and 11. Efficacy results are also shown for co-primary endpoints by Bio-IR and non-Bio-IR Sub-groups in Table 11(A) and (B).

TABLE 10
Efficacy Results for Co-primary and Secondary Efficacy Endpoints (ITT1A1) for US-specific Protocol
A1) Efficacy results for US-specific protocol following first interim database lock

| Withdrawal | RISA | RISA | Adjusted treatment Difference | |
|---|---|---|---|---|
| (PBO) | 180 mg | 360 mg | 95% CI³ | p-Value³ |

| | | (N = 164) | (N = 157) | | (N = 141) | RISA | | RISA | | RISA | RISA |
|---|---|---|---|---|---|---|---|---|---|---|---|
| | Endpoints² | % (n)⁴ or | % (n)⁴ or | | % (n)⁴ or | 180 mg - | | 360 mg - | | 180 mg vs. | 360 mg vs. |
| | All endpoints | LSMEAN | LSMEAN | | LSMEAN | Withdrawal | | Withdrawal | | Withdrawal | Withdrawal |
| | at Week 52 | (SE) | (SE) | | (SE) | (PBO) | | (PBO) | | (PBO) | (PBO) |
| Co- | Clinical remission | 40.9 (67) | 54.8 | (86) | 51.4 (73) | 14.1 | [4.3, 23.9] | 13.9 | [3.5, 24.2] | 0.005^{S} | 0.009^{S} |
| Primary | (per CDAI) | | | | | | | | | | |
| | Endoscopic response | 22.0 (36) | 46.5 | (73) | 45.9 (65) | 25.3 | [16.7, 34.0] | 27.1 | [18.0, 36.3] | <0.001^{S} | <0.001^{S} |
| Secondary | 1. Clinical remission | 39.6 (65) | 45.9 | (72) | 51.1 (72) | 7.1 | [−2.7, 16.9] | 14.4 | [4.2, 24.6] | 0.157^{NS} | 0.006^{S} |
| Endpoints: | (perSF/APS) | | | | | | | | | | |
| Sequential | 2. Maintenance of | N = 96 | N = 96 | | N = 81 | 14.5 | [1.5, 27.5] | 12.8 | [−1.1, 26.8] | 0.029^{NS} | 0.07^{NS} |
| | clinical remission (per | 56.3 (54) | 69.8 (67) | | 67.3 (55) | | | | | | |
| | CDAI) | | | | | | | | | | |
| | 3. Ulcer-free endoscopy | N = 162 | N = 157 | | N = 141 | 13.3 | [6.1, 20.5] | 21.4 | [13.7, 29.2] | <0.001^{NS} | <0.001^{NS} |
| | | 10.5 (17) | 23.6 (37) | | 30.0 (42) | | | | | | |
| | 4. Endoscopic | 12.8 (21) | 29.3 | (46) | 38.4 (54) | 16.6 | [8.8, 24.3] | 27.7 | [19.2, 36.3] | <0.001^{NS} | <0.001^{NS} |
| | remission | | | | | | | | | | |
| | 5. Change from baseline | N = 93 | N = 116 | | N = 102 | 0.4 | [−1.8, 2.7] | 0.5 | [−1.8, 2.8] | 0.700^{NS} | 0.677^{NS} |
| | of induction in FACIT- | 14.9 (0.86) | 15.4 (0.77) | | 15.4 (0.81) | | | | | | |
| | Fatigue | | | | | | | | | | |
| | 6. Steroid-free clinical | N = 51 | N = 51 | | N = 42 | 28.0 | [11.7, 44.3] | 3.2 | [−10.2, 16.5] | 0.001^{NS} | 0.644^{NS} |
| | remission (per CDAI) | 23.5 (12) | 51.0 (26) | | 25.7 (11) | | | | | | |
| | 7. CDAI clinical | 48.2 (79) | 66.2 | (104) | 60.9 (86) | 18.2 | [8.2, 28.2] | 15.5 | [5.0, 26.0] | <0.001^{NS} | 0.004^{NS} |
| | response | | | | | | | | | | |
| | 8. SF remission | 44.5 (73) | 51.0 | (80) | 56.2 (79) | 7.5 | [−2.5, 17.4] | 14.2 | [4.0, 24.5] | 0.143^{NS} | 0.007^{NS} |
| | 9. AP remission | 46.3 (76) | 56.1 | (88) | 55.8 (79) | 10.3 | [−0.0, 20.6] | 12.4 | [1.8, 23.0] | 0.050^{NS} | 0.021^{NS} |
| Secondary | CDAI clinical remission | 15.9 (26) | 37.6 | (59) | 35.1 (49) | 22.1 | [14.1, 30.1] | 22.3 | [13.5, 31.1] | <0.001^{NS} | <0.001^{NS} |
| Endpoints: | and endoscopic | | | | | | | | | | |
| Holm | response | | | | | | | | | | |
| Procedure | Deep remission (per | 10.4 (17) | 24.8 | (39) | 28.4 (40) | 14.3 | [7.1, 21.6] | 20.4 | [12.3, 28.6] | <0.001^{NS} | <0.001^{NS} |
| | CDAI) | | | | | | | | | | |
| | Exposure adjusted | 0.0580 (8) | 0.0346 | (5) | 0.0721 (9) | -2.34 | [−7.3789, 2.6982] | 1.41 | [−4.7875, 7.5989] | 0.363^{NS} | 0.656^{NS} |
| | occurrence of CD- | | | | | | | | | | |
| | related hospitalizations | | | | | | | | | | |
| | from Week 0 through | | | | | | | | | | |
| | Week 52 | | | | | | | | | | |

A2) Efficacy results for US-specific protocol following second interim database lock

| RISA | RISA | Adjusted treatment Difference | |
|---|---|---|---|
| 180 mg SC | 360 mg SC | 95% CI² | p-Value² |

| | | | | | | | |
|---|---|---|---|---|---|---|---|
| | Withdrawal/PBO | (N = 157) | (N = 141) | RISA | RISA | RISA | RISA |
| | (N = 164) | % (n)³ or | %(n)³ or | 180 mg SC- | 360 mg SC- | 180 mg SC vs. | 360 mg SC vs. |
| | % (n)³ or | LSMEAN | LSMEAN | Withdrawal/ | Withdrawal/ | Withdrawal/ | Withdrawal/ |
| Endpoints | LSMEAN (SE) | (SE) | (SE) | PBO | PBO | PBO | PBO |

| Co-Prim | Clinical remission | 40.9 (67) | 55.4 | (87) | 52.2 (74) | 14.7 | [5.0, 24.5] | 14.6 | [4.3,25.0] | 0.003^{S} | 0.005^{S} |
|---|---|---|---|---|---|---|---|---|---|---|---|
| | (per CDAI) at | | | | | | | | | | |
| | Week 52 | | | | | | | | | | |
| | Endoscopic response | 22.0 (36) | 47.1 | (74) | 46.5 (66) | 26.0 | [17.2, 34.7] | 27.8 | [18.7, 37.0] | <0.001^{S} | <0.001^{S} |
| | at Week 52 | | | | | | | | | | |
| 1 | Clinical remission | 39.6 (65) | 46.5 | (73) | 51.8 (73) | 7.7 | [−2.1, 17.5] | 15.2 | [4.9, 25.4] | 0.124^{NS} | 0.004^{S} |
| | (SF/APS) at Week 52 | | | | | | | | | | |
| 2 | Maintenance of | N = 96 | N = 96 | | N = 81 | 15.5 | [2.5, 28.5] | 14.3 | [0.5, 28.1] | 0.020^{NS} | 0.043^{NS} |
| | clinical remission | 56.3 (54) | 70.8 (68) | | 68.8 (56) | | | | | | |
| | (CDAI) at Week 52 | | | | | | | | | | |
| 3 | Ulcer-free | N = 162 | N = 157 | | N = 141 | 13.9 | [6.6, 21.2] | 22 | [14.3, 29.7] | <0.001^{NS} | <0.001^{NS} |
| | endoscopy | 10.5 (17) | 24.2 (38) | | 30.5 (43) | | | | | | |
| | at Week 52 | | | | | | | | | | |
| 4 | Endoscopic remission | 12.8 (21) | 29.9 | (47) | 39.1 (55) | 17.2 | [9.3, 25.0] | 28.5 | [19.9, 37.0] | <0.001^{NS} | <0.001^{NS} |
| | at Week 52 | | | | | | | | | | |
| 5 | Change from | N = 93 | N = 117 | | N = 104 | 0.5 | [−1.7, 2.7] | 0.4 | [−1.9, 2.7] | 0.663^{NS} | 0.703^{NS} |
| | baseline of the | 15.0 (0.86) | 15.5 (0.77) | | 15.4 (0.81) | | | | | | |
| | induction study in | | | | | | | | | | |
| | FACIT-Fatigue at | | | | | | | | | | |
| | Week 52 | | | | | | | | | | |
| 6 | Steroid-free clinical | N = 51 | N = 51 | | N = 42 | 29.6 | [13.5, 45.8] | 3.0 | [−10.3,16.4] | <0.001^{NS} | 0.655^{NS} |
| | remission (CDAI) | 23.5 (12) | 52.9 (27) | | 25.6 (11) | | | | | | |
| | at Week 52 | | | | | | | | | | |
| 7 | CDAI clinical | 48.2 (79) | 66.9 | (105) | 61.6 (87) | 18.8 | [8.8, 28.8] | 16.2 | [5.7, 26.6] | <0.001^{NS} | 0.002^{NS} |
| | response | | | | | | | | | | |
| | at Week 52 | | | | | | | | | | |
| 8 | SF remission | 44.5 (73) | 51.6 | (81) | 57.0 (80) | 8.1 | [−1.9, 18.0] | 15.0 | [4.8, 25.3] | 0.113^{NS} | 0.004^{NS} |
| | at Week 52 | | | | | | | | | | |
| 9 | AP remission | 46.3 (76) | 56.7 | (89) | 56.5 (80) | 10.9 | [0.6, 21.2] | 13.2 | [2.6, 23.8] | 0.037^{NS} | 0.014^{NS} |
| | at Week 52 | | | | | | | | | | |
| Holm | CDAI clinical | 15.9 (26) | 38.2 | (60) | 35.8 (50) | 22.7 | [14.6, 30.8] | 23.1 | [14.3, 31.9] | <0.001^{NS} | <0.001^{NS} |
| Procedure | remission and | | | | | | | | | | |
| | endoscopic response | | | | | | | | | | |
| | at Week 52 | | | | | | | | | | |
| | Deep remission | 10.4 (17) | 25.5 | (40) | 29.1 (41) | 14.9 | [7.6, 22.3] | 21.2 | [13.1, 29.4] | <0.001^{NS} | <0.001^{NS} |
| | (CDAI) at Week 52 | | | | | | | | | | |
| | Exposure adjusted | 0.058 (8) | 0.034 | (5) | 0.071 (9) | −2.4 | [−7.4, 2.7] | 1.3 | [−4.8, 7.5] | 0.355^{NS} | 0.676^{NS} |
| | occurrence of CD- | | | | | | | | | | |
| | related | | | | | | | | | | |
| | hospitalizations | | | | | | | | | | |
| | from Week 0 | | | | | | | | | | |
| | through Week 52 | | | | | | | | | | |

For Table A2:
1) ITT1A: randomized subjects who received at least one dose of study drug in M16-000 sub-study 1 who had baseline eligible SES-CD of ≥6 (≥4 for isolated ileal disease) and received IV risankizumab for only one period of 12 weeks in the induction study (M16-006 or M15-991).
2Adjusted treatment difference, 95% CI and p-values for comparison of binary endpoints between RISA and PBO were calculated using Cochran-Mantel-Haenszel (CMH) test adjusted for randomization stratification factors (endoscopic response (yes or no, per local read), clinical remission status (yes or no) from the last visit of the induction study, as well as by last IV dose during risankizumab induction periods (1200 mg or 600 mg)); 95% CI and p-values for comparison of continuous endpoints between RISA and PBO were calculated using ANCOVA with baseline, treatment, visit, treatment by visit interaction and stratification factors in the model.
³The % (n) represents the synthesized results from multiple imputation.
^{NS}The endpoint achieved or did not achieve, statistical significance based on the pre-specified graphical testing procedure for the US-specific protocol

TABLE 11(A)
Co-primary Endpoints Efficacy Results for Bio-IR Sub-group

| | | | | Treatment Difference | |
|---|---|---|---|---|---|
| | | | | 95% CI | |
| | Withdrawal | RISA | RISA | RISA | RISA |
| | (PBO) | 180 mg | 360 mg | 180 mg - | 360 mg - |
| | (N = 123) | (N = 113) | (N = 102) | Withdrawal | Withdrawal |
| Endpoints | n (%) | n (%) | n (%) | (PBO) | (PBO) |
| The achievement | 43 (35.0) | 55 (48.7) | 48 (46.6) | 13.7[1.2, 26.2] | 11.6[−1.3, 24.5] |
| of clinical | | | | | |
| remission (CDAI) | | | | | |
| at Week 52 | | | | | |
| The achievement | 25 (20.3) | 46 (40.7) | 44 (42.8) | 20.4[8.9, 31.9] | 22.5[10.5, 34.5] |
| of endoscopic | | | | | |
| response at Week | | | | | |
| 52 | | | | | |
| The achievement | 42 (34.1) | 46 (40.7) | 48 (47.1) | 6.6[−5.8, 18.9] | 12.9[0.1, 25.8] |
| of clinical | | | | | |
| remission | | | | | |
| (SF/APS) at Week | | | | | |
| 52 | | | | | |

TABLE 11(B)
Co-primary Endpoints Efficacy Results for non-Bio-IR Sub-group

| | | | | Treatment Difference | |
|---|---|---|---|---|---|
| | | | | 95% CI | |
| | Withdrawal | RISA | RISA | RISA | RISA |
| | (PBO) | 180 mg | 360 mg | 180 mg - | 360 mg - |
| | (N = 41) | (N = 44) | (N = 39) | Withdrawal | Withdrawal |
| Endpoints | n (%) | n (%) | n (%) | (PBO) | (PBO) |
| The achievement | 24 (58.5) | 31 (70.5) | 25 (64.1) | 11.9[−8.3, 32.1] | 5.6[−15.7, 26.9] |
| of clinical | | | | | |
| remission (CDAI) | | | | | |
| at Week 52 | | | | | |
| The achievement | 11 (26.8) | 27 (61.4) | 21 (53.8) | 34.5[14.8, 54.3] | 27.0[6.3, 47.7] |
| of endoscopic | | | | | |
| response at | | | | | |
| Week 52 | | | | | |
| The achievement | 23 (56.1) | 26 (59.1) | 24 (61.5) | 3.0[−18.0, 24.0] | 5.4[−16.1, 27.0] |
| of clinical | | | | | |
| remission | | | | | |
| (SF/APS) at Week | | | | | |
| 52 | | | | | |

Pharmacokinetics and Immunogenicity:

Serum risankizumab concentrations were summarized at each time point for each dosing regimen using descriptive statistics.

ADA incidence was summarized by cohorts and study visits. ADA titers were tabulated for each subject at the respective study visits.

Adverse Events

Adverse events (AE's) and serious adverse events (SAEs), laboratory data, and vital signs were the primary safety parameters in this study. All safety comparisons were performed between treatment groups using the safety set. Treatment emergent AEs were defined as events that begin or worsen either on or after the first dose of the study drug and within 140 days after the last dose of the study drug for subjects who discontinued the study prematurely or completed Sub-study 1 or 2 and did not enter Sub-study 3.

An overview of treatment-emergent AEs, including AEs of special interest such as serious infection, malignancies, major adverse cardiovascular events, systemic hypersensitivity reactions/infusion reactions, AEs leading to death and adverse events leading to premature discontinuation (see details in the SAP), AEs by Medical Dictionary for Drug Regulatory Activities (MedDRA version 18.1 or later) preferred term and system organ class, AEs by maximum relationship to study drug, and AEs by maximum severity were summarized by number and percentage.

Changes in laboratory data were described using statistical characteristics and comparison between treatment groups was performed using a one-way Analysis of Variance (ANOVA). In addition, shift tables and listings were provided for abnormal values, whereby the normal range of the analyzing laboratory was used. Vital signs were analyzed similarly.

In sub-study 1 the overall incidences of treatment-emergent AEs and SAEs were similar among RISA and withdrawal (PBO)treatment groups in the 52-Week maintenance Period. Similarly, the incidences of AEs in areas of safety interest (ASI) were generally similar across treatment groups.
The incidences of serious infection events were 2.8% and 4.5%, in RISA 180 mg SC group and 360 mg SC group compared to 3.8% in the withdrawal (PBO)group.
The rates of hepatic events were 2.8% and 3.9% in RISA 180 mg SC group and 360 mg SC group compared to 2.2% in placebo group, with non-serious, most being liver enzyme increase and majority having no change in study drug treatment.
The rates of injection site reaction, including infusion reaction, were generally comparable across treatment groups with all events mild or moderate.
No events of adjudicated anaphylactic reaction and active TB were reported in any treatment group. One event of active TB was reported in sub-study 2.
Two events of malignant tumors were reported in the randomized treatment groups, one in the withdrawal (PBO) group, the other one in RISA 360 mg SC group.
Two events of adjudicated MACE were reported in the randomized treatment groups, one in the withdrawal (PBO) group, the other one in RISA 360 mg SC group. One event of adjudicated MACE was reported in the non-randomized RISA 180 mg SC group.

The most common TEAEs in any treatment groups were Crohn's disease, nasopharyngitis, arthralgia, headache, abdominal pain, anaemia, nausea, and diarrhea; no deaths were reported.

Study Impact

The pivotal portion of this sub-study 1 was a re-randomized responder withdrawal design. The efficacy objective was to compare the efficacy of subjects who responded to IV RISA and continued to receive RISA SC in maintenance with subjects who went through a withdrawal period. High rates of clinical remission were observed for subjects who continued to receive RISA SC treatment. A clear treatment effect was observed with the continuation of RISA treatment versus withdrawal from RISA for objective endpoints (e.g., endoscopic endpoints). Durability of RISA's efficacy was demonstrated with a high proportion of subjects (63-70%) maintaining clinical remission over the course of the 52-week study. Finally, a high proportion of subjects who continued with RISA treatment were able to achieve endoscopic remission, ulcer-free endoscopy, and the stringent endpoint of deep remission (i.e., clinical remission and endoscopic remission in the same subject), which are all important treatment goals and have been associated with better long-term outcomes. RISA as maintenance treatment for 1-year was generally safe and well-tolerated. In subjects who achieved clinical response to 12 weeks of IV RISA induction and received 52 weeks of RISA SC maintenance treatment, both RISA 180 mg SC and 360 mg achieved significantly higher rates of clinical remission (per CDAI and SF/APS) and endoscopic response compared to PBO. Durability of efficacy was demonstrated with a high proportion of subjects maintaining clinical remission over the course of the 52-week study. In addition, a high proportion of subjects were able to taper off corticosteroids and achieve clinical remission. Finally, a high proportion of subjects were able to achieve endoscopic remission, ulcer-free endoscopy, and the stringent endpoint of deep remission (clinical and endoscopic remission in the same subject). RISA as maintenance treatment for 1-year was generally safe and well tolerated.

Taken together, the results from the RISA induction and maintenance studies align with recent STRIDE-Il (Selecting Therapeutic Targets in IBD) IOIBD guidelines, which recommend short term treatment goals of symptomatic response, intermediate treatment goals of symptomatic remission and normalization of hs-CRP and fecal markers, and long-term treatment goals of clinical remission and endoscopic healing (Turner D, et al. Gastro 2021).

### Example 6: Results of the 12-Week Induction Period of Study M16-006

Study M16-006 was a Phase 3 study to evaluate the efficacy and safety of risankizumab (RISA) as the induction therapy in subjects with moderately to severely active Crohn's disease (CD). It consisted of two periods: a 12-Week, randomized, double-blind, placebo-controlled induction period (12-Week Induction Period) and the exploratory Induction Period 2 for subjects who did not achieve clinical response at Week 12. The results from the 12-Week Induction Period are shown as follows.

Demographic, Baseline Characteristics, and Subject Disposition

A total of 931 subjects were randomized in the study, where 855 subjects had baseline eligible SES-CD of [symbol] 6 ( [symbol] 4 for isolated ileal disease), and an additional 76 subjects had a lower baseline eligible SES-CD. All randomized subjects received at least one dose of study drug in the 12-Week Induction Period. The intent-to-treat population for efficacy analysis in this period (ITT1A) comprised of a total of 850 subjects (336* subjects in the RISA 600 mg IV group, 339* subjects in the RISA 1200 mg IV group and 175* subjects in the PBO group). There were 488 subjects from Bio-IR population and 362 subjects from non-Bio-IR population. Key demographics and baseline characteristics were generally balanced between three treatment groups. Discontinuation rates were similar among three treatment groups. *5 subjects (2 subjects in the RISA 1200 mg IV group, 2 subjects in the RISA 600 mg IV group and 1 subject in the PBO group) were excluded from the efficacy analysis from a significant non-compliance site. These subjects were included in the safety analysis.

Efficacy:

The study met the co-primary endpoints and majority of the multiplicity-controlled secondary endpoints demonstrating superiority of RISA 600 mg and RISA 1200 mg intravenous (IV) dose comparing to placebo (PBO) for both US-specific protocol and global protocol outside US.

For US-specific protocol co-primary endpoints:

For the US-specific protocol, the study met the co-primary endpoints for both RISA 600 mg and 1200 mg IV dose groups comparing to the PBO group (all p-values <0.001 for clinical remission (CDAI) and all p-values <0.001 for endoscopic response). The multiplicity-controlled secondary endpoints that met or did not meet statistical significance per statistical testing procedure were presented in Table 12(A1) and Table 12(A2).

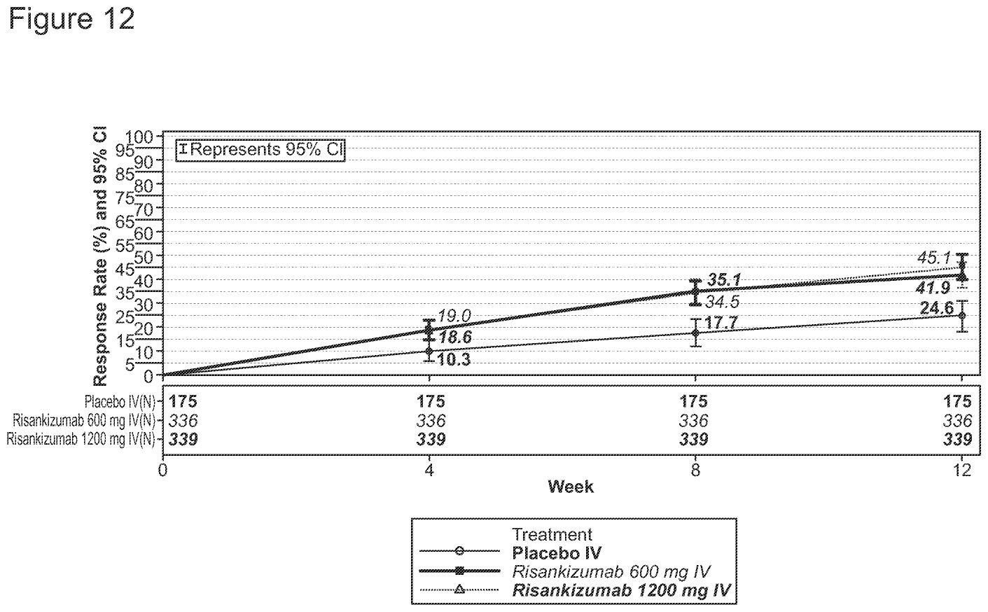

The clinical remission evaluated by Crohn's disease activity index (CDAI) rate at Week 12 was significantly higher in the RISA 600 mg and RISA 1200 mg IV induction doses (45.1% and 41.9%, respectively) compared to the PBO (24.6%) with p-value <0.001 for both dose groups (see FIG. 12).

The endoscopic response rate at Week 12 was significantly higher in the RISA 600 mg and RISA 1200 mg IV induction doses (40.3% and 32.2%, respectively) compared to the PBO (12.0%) with p-value <0.001 for both dose groups.

For global protocol outside US co-primary endpoints:

For the global protocol outside US, the study met the co-primary endpoints for both RISA 600 mg and 1200 mg IV dose groups comparing to the PBO group (all p-values <0.001 for clinical remission (SF/APS) and all p-values <0.001 for endoscopic response). The multiplicity-controlled secondary endpoints that met or did not meet statistical significance per statistical testing procedure were presented in Table 12(B1) and Table (B2).

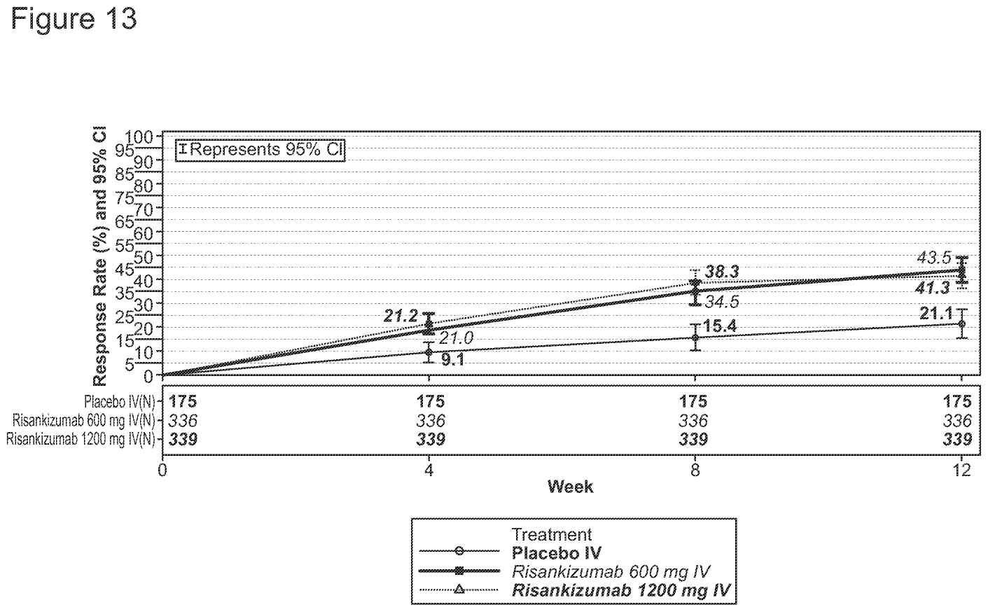

The clinical remission (SF/APS*) rate at Week 12 was significantly higher in the RISA 600 mg and RISA 1200 mg IV induction doses (43.5% and 41.3%, respectively) compared to the PBO (21.1%) with p-value <0.001 for both dose groups (see FIG. 13). *SF/APS: stool frequency and abdominal pain which are components of CDAI as patient reported outcome (i.e., PRO2).

The endoscopic response rate at Week 12 was significantly higher in the RISA 600 mg and RISA 1200 mg IV induction dose (40.3% and 32.2%, respectively) compared to the PBO (12.0%) with p-value <0.001 for both dose groups.

Clinical response and remission were seen as early as Week 4. Clinical remission rates over time are shown in FIG. 12 and FIG. 13. Efficacy results by Bio-IR and non-Bio-IR populations were included in Tables 13 and 14.

TABLE 12
Efficacy Results for Co-primary and Secondary Efficacy Endpoints (ITT1A¹)

| A1) Efficacy results for US-specific protocol following first interim database lock | | | | | |
|---|---|---|---|---|---|
| | | RISA | RISA | | |
| | PBO | 600 mg IV | 1200 mg IV | Adjusted treatment Difference | |
| | (N = 175) | (N = 336) | (N = 339) | 95% CI³ | p-Value³ |

| | | % (n)⁴ or | % (n)⁴ or | % (n)⁴ or | | □□(RISA | | □□(RISA | | RISA | RISA |
|---|---|---|---|---|---|---|---|---|---|---|---|
| | | LSMEAN | LSMEAN | LSMEAN | | 600 mg | | 1200 mg | | 600 mg | 1200 mg |
| | Endpoints² | (SE) | (SE) | (SE) | | IV-PBO) | | IV-PBO) | | IV vs. PBO | IV vs. PBO |
| Primary | The achievement of CDAI clinical | 24.6% (43) | 45.1% (152) | 41.9% | (142) | 20.8% | [12.5%, 29.1%] | 17.1% | [8.9%, 25.3%] | <0.001^{S} | <0.001^{S} |
| | remission at Week 12 | | | | | | | | | | |
| | The achievement of endoscopic | 12.0% (21) | 40.3% (135) | 32.2% | (109) | 28.3% | [21.2%, 35.3%] | 20.1% | [13.3%, 26.8%] | <0.001^{S} | <0.001^{S} |
| | response at Week 12 | | | | | | | | | | |
| Secondary | 1. The achievement of clinical | 21.1% (37) | 43.5% (146) | 41.3% | (140) | 22.5% | [14.5%, 30.6%] | 19.7% | [11.8%, 27.7%] | <0.001^{S} | <0.001^{S} |
| Endpoints: | remission at Week 12 | | | | | | | | | | |
| Sequential | 2. The achievement of CDAI | 25.2% (44) | 40.8% (137) | 36.9% | (125) | 15.3% | [7.0%, 23.5%] | 10.9% | [2.8%, 19.0%] | <0.001^{S} | 0.008^{S} |
| | clinical response at Week 4 | | | | | | | | | | |
| | 3. The achievement of CDAI | 36.2% (63) | 61.7% (207) | 65.2% | (221) | 25.7% | [17.0%, 34.5%] | 28.6% | [19.9%, 37.3%] | <0.001^{S} | <0.001^{S} |
| | clinical response at Week 12 | | | | | | | | | | |
| | 4. Change from baseline in | (N = 134) | (N = 302) | (N = 309) | | 5.2 | [3.2, 7.2] | 4.1 | [2.1, 6.1] | <0.001^{S} | <0.001^{S} |
| | FACIT- Fatigue at Week 12 | 6.0 (0.86) | 11.2 (0.59) | 10.1 (0.59) | | | | | | | |
| | 5. The achievement of clinical | 10.3% (18) | 19.0% (64) | 18.6% | (63) | 8.1% | [1.9%, 14.2%] | 8.2% | [2.1%, 14.3%] | 0.010^{S} | 0.008^{S} |
| | remission (CDAI) at Week 4 | | | | | | | | | | |
| | 6. The achievement of CDAI | 5.7% (10) | 30.6% (103) | 23.0% | (78) | 25.2% | [19.1%, 31.2%] | 17.2% | [11.7%, 22.8%] | <0.001^{S} | <0.001^{S} |
| | clinical response and | | | | | | | | | | |
| | endoscopic response at Week 12 | | | | | | | | | | |
| | 7. The achievement of SF | 28.6% (50) | 54.2% (182) | 54.3% | (184) | 25.5% | [17.1%, 34.0%] | 25.1% | [16.7%, 33.5%] | <0.001^{S} | <0.001^{S} |
| | remission at Week 12 | | | | | | | | | | |
| | 8. The achievement of AP | 37.8% (66) | 59.3% (199) | 58.1% | (197) | 21.7% | [12.9%, 30.5%] | 19.8% | [11.0%, 28.6%] | <0.001^{S} | <0.001^{S} |
| | remission at Week 12 | | | | | | | | | | |
| Secondary | The achievement of endoscopic | 9.1% (16) | 24.2% (81) | 23.9% | (81) | 15.0% | [8.9%, 21.2%] | 15.0% | [9.0%, 21.0%] | <0.001^{S} | <0.001^{S} |
| Endpoints: | remission at Week 12 | | | | | | | | | | |
| Holm | The achievement of enhanced | 31.5% (55) | 46.9% (158) | 43.4% | (147) | 15.5% | [6.8%, 24.1%] | 11.4% | [2.8%, 20.0%] | <0.001^{S} | 0.009^{S} |
| Procedure | clinical response at Week 4 | | | | | | | | | | |
| | The achievement of ulcer-free | (N = 173) | (N = 336) | (N = 338) | | 13.7% | [7.9%, 19.5%] | 8.9% | [3.5%, 14.3%] | <0.001^{S} | 0.001^{S} |
| | endoscopy at Week 12 | 7.5% (13) | 21.0% (71) | 16.3% (55) | | | | | | | |
| | The achievement of enhanced | 40.6% (71) | 63.1% (212) | 64.3% | (218) | 22.7% | [13.9%, 31.5%] | 23.0% | [14.2%, 31.7%] | <0.001^{S} | <0.001^{S} |
| | clinical response at Week 12 | | | | | | | | | | |
| | The achievement of resolution of | (N = 64) | (N = 140) | (N = 158) 43.7% | | 15.3% | [3.0%, 27.6%] | 24.4% | [12.0%, 36.7%] | 0.015^{NS} | <0.001^{S} |
| | EIMs at Week 12, in subjects with | 20.5% (13) | 38.1% (53) | (69) | | | | | | | |
| | any EIMs at baseline | | | | | | | | | | |
| | Occurrence of CD-related | 12.0% (21) | 3.3% (11) | 1.8% | (6) | −8.7% | [−13.9%, −3.5%] | −10.2% | [−15.2%, −5.2%] | <0.001^{S} | <0.001^{S} |
| | hospitalization through Week 12 | | | | | | | | | | |

| | | | | | | | | | |
|---|---|---|---|---|---|---|---|---|---|
| The achievement of no draining | (N = 9) | (N = 18) | (N = 24) | 5.6% | [−28.6%, 39.7%] | 6.9% | [−25.7%, 39.6%] | 1.00^{NS} | 1.00^{NS} |
| fistulas at Week 12 in subjects | 22.2% (2) | 27.8% (5) | 29.2% (7) | | | | | | |
| with draining fistulas at baseline | | | | | | | | | |

¹1TT1A: randomized subjects who received at least one dose of study drug in the 12-Week Induction Period who had baseline eligible SES-CD of ≥6 (≥4 for isolated ileal disease).
²Definition of the endpoints are included in Example 4.
³Adjusted treatment difference, 95% CI and p-values for comparison of binary endpoints between RISA and PBO were calculated using Cochran-Mantel-Haenszel (CMH) test adjusted for randomization stratification
factors (the number of prior biologies failed (0, 1, >1) and baseline corticosteroid use (yes or no)); 95% CI and p-values for comparison of continuous endpoints between RISA and PBO were calculated using MMRM
(mixed effect model repeat measurement) with baseline, treatment, visit, treatment by visit interaction and stratification factors in the model.
⁴The % (n) represents the synthesized results from multiple imputation.
^{S, NS}The endpoint achieved or did not achieve, statistical significance based on the pre-specified graphical testing procedure for the US-specific protocol.

A2) Efficacy results for US-specific protocol following second interim database lock

| | | | | RISA | | RISA | | | |
|---|---|---|---|---|---|---|---|---|---|
| | | PBO | | 600 mg IV | | 1200 mg IV | | | |
| | | (N = 175) | | (N = 336) | | (N = 339) | | p-Value² | |
| | | % (n)¹ or | | % (n)¹ or | | % (n)¹ or | | RISA | RISA |
| | | LSMEAN | | LSMEAN | | LSMEAN | | 600 mg | 1200 mg |
| Endpoints | | (SE) | | (SE) | | (SE) | | IV vs. PBO | IV vs. PBO |
| 1 | Clinical remission (PRO2) at Week 12 | 21.7% | (38) | 43.5% | (146) | 41.0% | (139) | <0.001 | <0.001 |
| 2 | CDAI clinical response at Week 4 | 25.2% | (44) | 40.8% | (137) | 37.2% | (126) | <0.001 | <0.001 |
| 3 | CDAI clinical response at Week 12 | 37.3% | (65) | 60.0% | (202) | 64.9% | (220) | <0.001 | <0.001 |
| 4 | Change from baseline in FACIT-Fatigue at Week 12 | 6.0 | (0.86) | 11.2 | (0.59) | 10.1 | (0.59) | <0.001 | <0.001 |
| 5 | Clinical remission (CDAI) at Week 4 | 10.3% | (18) | 18.4% | (62) | 18.9% | (64) | 0.015 | 0.007 |
| 6 | CDAI clinical response and endoscopic response at | 5.7% | (10) | 30.3% | (102) | 23% | (78) | <0.001 | <0.001 |
| | Week12 | | | | | | | | |
| 7 | SF remission at Week 12 | 29.8% | (52) | 54.2% | (182) | 54.0% | (183) | <0.001 | <0.001 |
| 8 | AP remission at Week 12 | 38.5% | (67) | 59.6% | (200) | 58.1% | (197) | <0.001 | <0.001 |
| Holm | Endoscopic remission at Week 12 | 9.1% | (16) | 24.2% | (81) | 23.9% | (81) | <0.001 | <0.001 |
| Procedure | Enhanced clinical response (PRO2) at Week 4 | 31.0% | (54) | 46.0% | (155) | 43.4% | (147) | <0.001 | 0.007 |
| | Ulcer-free endoscopy at Week 12 | N = 173 | | N = 336 | | N = 338 | | <0.001 | 0.001 |
| | | 7.5% (13) | | 21% (71) | | 16.3% (55) | | | |
| | Enhanced clinical response (PRO2) at Week 12 | 42.0% | (73) | 62.8% | (211) | 64.3% | (218) | <0.001 | <0.001 |
| | Resolution of EIMs at Week 12, in subjects with any | N = 64 | | N = 140 | | N = 158 | | 0.015^{NS} | <0.001 |
| | EIMs at baseline | 20.5% (13) | | 38.1% (53) | | 43.7% (69) | | | |
| | CD-related hospitalization through Week 12 | 12.0% | (21) | 3.3% | (11) | 1.8% | (6) | <0.001 | <0.001 |

| | | | | | |
|---|---|---|---|---|---|
| No draining fistulas at Week 12 in subjects with | N = 9 | N = 18 | N = 24 | 1.00^{NS} | 1.00^{NS} |
| draining fistulas at baseline | 22.2% (2) | 27.8% (5) | 29.2% (7) | | |

¹The % (n) represents the synthesized results from multiple imputation.
²Adjusted treatment difference, 95% CI and p-values for comparison of binary endpoints between RISA and placebo were calculated using Cochran-Mantel-Haenszel (CMH) test adjusted for randomization
stratification factors (the number of prior biologies failed and baseline corticosteroid use); 95% CI and p-values for comparison of continuous endpoints between RISA and placebo were calculated using MMRM
(mixed effect model repeat measurement) with baseline, treatment, visit, treatment by visit interaction and stratification factors in the model.
^{NS}Not statistically significant based on adjusted p-value from the graphical testing procedure
ITT1A: includes randomized subjects who received at least one dose of study drug who had baseline eligible SES-CD of ≥6 (≥4 for isolated ileal disease), excluding subjects from the non-compliant site
(Investigator ID 527969).
See Backup slide for graphical testing procedure

| B1) Efficacy results for global protocol outside US following first interim database lock | | | | | |
|---|---|---|---|---|---|
| | | RISA | RISA | | |
| | | 600 mg IV | 1200 mg IV | Adjusted treatment Difference | |
| | PBO (N = 175) | (N = 336) | (N = 339) | 95% CI³ | p-Value³ |

| | | % (n)⁴ or | | % (n)⁴ or | | % (n)⁴ or | | (RISA | | (RISA | | RISA | RISA |
|---|---|---|---|---|---|---|---|---|---|---|---|---|---|
| | | LSMEAN | | LSMEAN | | LSMEAN | | 600 mg | | 1200 mg | | 600 mg | 1200 mg |
| | End points² | (SE) | | (SE) | | (SE) | | IV-PBO) | | IV-PBO) | | IV vs. PBO | IV vs. PBO |
| Primary | The achievement of clinical | 21.1% | (37) | 43.5% | (146) | 41.3% | (140) | 22.5% | [14.5%, 30.6%] | 19.7% | [11.8%, 27.7%] | <0.001^{S} | <0.001^{S} |
| | remission (PR02) at Week 12 | | | | | | | | | | | | |
| | The achievement of endoscopic | 12.0% | (21) | 40.3% | (135) | 32.2% | (109) | 28.3% | [21.2%, 35.3%] | 20.1% | [13.3%, 26.8%] | <0.001^{S} | <0.001^{S} |
| | response at Week 12 | | | | | | | | | | | | |
| Secondary | 1. The achievement of clinical remission | 24.6% | (43) | 45.1% | (152) | 41.9% | (142) | 20.8% | [12.5%, 39.1%] | 17.1% | [8.9%, 25.3%] | <0.001^{S} | <0.001^{S} |
| | (CDAI) at Week 12 | | | | | | | | | | | | |
| | 2. The achievement of CDAI clinical | 25.2% | (44) | 40.8% | (137) | 36.9% | (125) | 15.3% | [7.0%, 23.5%] | 10.9% | [2.8%, 19.0%] | <0.001^{S} | 0.008^{S} |
| | response at Week 4 | | | | | | | | | | | | |
| | 3. The achievement of clinical remission | 9.1% | (16) | 21.0% | (71) | 21.2% | (72) | 11.5% | [5.5%, 17.6%] | 11.8% | [5.8%, 17.9%] | <0.001^{S} | <0.001^{S} |
| | at Week 4 | | | | | | | | | | | | |
| | 4. The achievement of CDAI clinical | 36.2% | (63) | 61.7% | (207) | 65.2% | (221) | 25.7% | [17.0%, 34.5%] | 28.6% | [19.9%, 37.3%] | <0.001^{S} | <0.001^{S} |
| | response at Week 12 | | | | | | | | | | | | |
| | 5. Change from baseline in FACIT- | (N = 134) | | (N = 302) | | (N = 309) | | 5.2 | [3.2, 7.2] | 4.1 | [2.1, 6.1] | <0.001^{S} | <0.001^{S} |
| | Fatigue at Week 12 | 6.0 (0.86) | | 11.2 (0.59) | | 10.1 (0.59) | | | | | | | |
| | 6. Change from baseline in IBDQ total | (N = 134) | | (N = 302) | | (N = 309) | | 20.7 | [14.4, 27.1] | 19.5 | [13.1, 25.8] | <0.001^{S} | <0.001^{S} |
| | score at Week 12 | 23.6 (2.73) | | 44.3 (1.87) | | 43.1 (1.85) | | | | | | | |
| Secondary | The achievement of enhanced clinical | 8% | (14) | 30.9% | (104) | 23.3% | (79) | 23.2% | [16.8%, 29.6%] | 15.1% | [9.2%, 21.1%] | <0.001^{S} | <0.001^{S} |
| Endpoints: | response and endoscopic response at | | | | | | | | | | | | |
| Holm | Week 12 | | | | | | | | | | | | |
| Procedure | The achievement of endoscopic remission | 9.1% | (16) | 24.2% | (81) | 23.9% | (81) | 15.0% | [8.9%, 21.2%] | 15.0% | [9.0%, 21.0%] | <0.001^{S} | <0.001^{S} |
| | at Week 12 | | | | | | | | | | | | |
| | The achievement of enhanced clinical | 31.5% | (55) | 46.9% | (158) | 43.4% | (147) | 15.5% | [6.8%, 24.1%] | 11.4% | [2.8%, 20.0%] | <0.001^{S} | 0.009^{S} |
| | response at Week 4 | | | | | | | | | | | | |
| | The achievement of ulcer-free | (N = 173) | | (N = 336) | | (N = 338) | | 13.7% | [7.9%, 19.5%] | 8.9% | [3.5%, 14.3%] | <0.001^{S} | 0.001^{S} |
| | endoscopy at Week 12 | 7.5% (13) | | 21.0% (71) | | 16.3% (55) | | | | | | | |
| | The achievement of enhanced clinical | 40.6% | (71) | 63.1% | (212) | 64.3% | (218) | 22.7% | [13.9%, 31.5%] | 23.0% | [14.2%, 31.7%] | <0.001^{S} | <0.001^{S} |
| | response at Week 12 | | | | | | | | | | | | |
| | The achievement of resolution of EIMs at | (N = 64) | | (N = 140) | | (N = 158) | | 15.3% | [3.0%, 27.6%] | 24.4% | [12.0%, 36.7%] | 0.015^{NS} | <0.001^{S} |
| | Week 12, in subjects with any EIMs at | 20.5% (13) | | 38.1% (53) | | 43.7% (69) | | | | | | | |
| | baseline | | | | | | | | | | | | |
| | Occurrence of CD-related hospitalization | 12.0% | (21) | 3.3% | (11) | 1.8% | (6) | −8.7% | [−13.9%, −3.5%] | −10.2% | [−15.2%, −5.2%] | <0.001^{S} | <0.001^{S} |
| | through Week 12 | | | | | | | | | | | | |

| | | | | | | | | | |
|---|---|---|---|---|---|---|---|---|---|
| The achievement of no draining fistulas at | (N = 9) | (N = 18) | (N = 24) | 5.6% | [−28.6%, 39.7%] | 6.9% | [−25.7%, 39.6%] | 1.00^{NS} | 1.00^{NS} |
| Week 12 in subjects with draining fistulas | 22.2% (2) | 27.8% (5) | 29.2% (7) | | | | | | |
| at baseline | | | | | | | | | |
| Change from baseline in WPAI- CD Overall | (N = 65) | (N = 156) | (N = 162) | −9.6 | [−17.9, −1.3] | −12.2 | [−20.4, −3.9] | 0.024^{NS} | 0.004^{S} |
| Work Impairment at Week 12 | −8.4 (3.56) | −17.9 (2.35) | −20.5 (2.31) | | | | | | |
| Change from baseline in SF-36 Physical | (N = 134) | (N = 302) | (N = 308) | 2.9 | [1.5, 4.3] | 3.3 | [1.9, 4.7] | <0.001^{S} | <0.001^{S} |
| Component Summary score at Week 12 | 5.5 (0.60) | 8.4 (0.41) | 8.8 (0.41) | | | | | | |

¹1TT1A: randomized subjects who received at least one dose of study drug in the 12-Week Induction Period who had baseline eligible SES-CD of ≥6 (≥4 for isolated ileal disease).
²Definition of the endpoints are included in Example 4.
³Adjusted treatment difference, 95% CI and p-values for comparison of binary endpoints between RISA and PBO were calculated using Cochran-Mantel-Haenszel (CMH) test adjusted for randomization stratification
factors (the number of prior biologies failed (0, 1, >1) and baseline corticosteroid use (yes or no)); 95% CI and p-values for comparison of continuous endpoints between RISA and PBO were calculated using MMRM
(mixed effect model repeat measurement) with baseline, treatment, visit, treatment by visit interaction and stratification factors in the model.
⁴The % (n) represents the synthesized results from multiple imputation.
^{S, NS}The endpoint achieved or did not achieve, statistical significance based on the pre-specified graphical testing procedure for the global protocol outside US.

B2) Efficacy results for global protocol outside US following second interim database lock

| | | | | RISA | | RISA | | | |
|---|---|---|---|---|---|---|---|---|---|
| | | PBO | | 600 mg IV | | 1200 mg IV | | | |
| | | (N = 175) | | (N = 336) | | (N = 339) | | p-Value² | |
| | | % (n)¹ or | | % (n)¹ or | | % (n)¹ or | | RISA | RISA |
| | | LSMEAN | | LSMEAN | | LSMEAN | | 600 mg | 1200 mg |
| Endpoints | | (SE) | | (SE) | | (SE) | | IV vs. PBO | IV vs. PBO |
| 1 | Clinical remission (CDAI) at Week 12 | 25.2% | (44) | 45.2% | (152) | 41.6% | (141) | <0.001 | <0.001 |
| 2 | CDAI clinical response at Week 4 | 25.2% | (44) | 40.8% | (137) | 37.2% | (126) | <0.001 | 0.007 |
| 3 | Clinical remission (PRO2) at Week 4 | 9.1% | (16) | 21% | (71) | 21.2% | (72) | <0.001 | <0.001 |
| 4 | CDAI clinical response at Week 12 | 37.3% | (65) | 60.0% | (202) | 64.9% | (220) | <0.001 | <0.001 |
| 5 | Change from baseline in FACIT-Fatigue at Week 12 | 6.0 | (0.86) | 11.2 | (0.59) | 10.1 | (0.59) | <0.001 | <0.001 |
| 6 | Change from baseline in IBDQ total score at Week 12 | 23.6 | (2.73) | 44.3 | (1.87) | 43.1 | (1.85) | <0.001 | <0.001 |
| Holm | Enhanced clinical response (PRO2) and endoscopic | 8% | (14) | 30.9% | (104) | 23.3% | (79) | <0.001 | <0.001 |
| Procedure | response at Week 12 | | | | | | | | |
| | Endoscopic remission at Week 12 | 9.1% | (16) | 24.2% | (81) | 23.9% | (81) | <0.001 | <0.001 |
| | Enhanced clinical response (PRO2) at Week 4 | 31.0% | (54) | 46.0% | (155) | 43.4% | (147) | <0.001 | 0.007 |
| | Ulcer-free endoscopy at Week 12 | (N = 173) | | (N = 336) | | (N = 338) | | <0.001 | 0.001 |
| | | 7.5% (13) | | 21% (71) | | 16.3% (55) | | | |
| | Enhanced clinical response (PRO2) at Week 12 | 42.0% | (73) | 62.8% | (211) | 64.3% | (218) | <0.001 | <0.001 |
| | Resolution of EIMs at Week 12, in subjects with any | N = 64 | | N = 140 | | N = 158 | | 0.020^{NS} | <0.001 |
| | EIMs at baseline | 20.5% (13) | | 38.1% (53) | | 43.7% (69) | | | |
| | CD-related hospitalization through Week 12 | 12.0% | (21) | 3.3% | (11) | 1.8% | (6) | <0.001 | <0.001 |
| | No draining fistulas at Week 12 in subjects with draining | (N = 9) | | (N = 18) | | (N = 24) | | 1.00^{NS} | 1.00^{NS} |
| | fistulas at baseline | 22.2% (2) | | 27.8% (5) | | 29.2% (7) | | | |
| | Change from baseline in WPAI-CD Overall Work | −8.4 | (3.56) | −17.9 | (2.35) | −20.5 | (2.31) | 0.024^{NS} | 0.004 |
| | Impairment at Week 12 | | | | | | | | |
| | Change from baseline in SF-36 Physical Component | 5.5 | (0.60) | 8.4 | (0.41) | 8.8 | (0.41) | <0.001 | <0.001 |
| | Summary score at Week 12 | | | | | | | | |

¹The % (n) represents the synthesized results from multiple imputation.
²Adjusted treatment difference, 95% CI and p-values for comparison of binary endpoints between RISA and placebo were calculated using Cochran-Mantel-Haenszel (CMH) test adjusted for randomization stratification
factors (the number of prior biologies failed and baseline corticosteroid use); 95% CI and p-values for comparison of continuous endpoints between RISA and placebo were calculated using MMRM (mixed effect model
repeat measurement) with baseline, treatment, visit, treatment by visit interaction and stratification factors in the model.
ITT1A: includes randomized subjects who received at least one dose of study drug who had baseline eligible SES-CD of ≥6 (≥4 for isolated ileal disease), excluding subjects from the non-compliant site
(Investigator ID 527969).
^{NS}Not statistically significant based on adjusted p-value from the graphical testing procedure
See Backup slide for graphical testing procedure

### Efficacy Results for Co-Primary Endpoints by Bio-IR and Non-Bio-IR Populations

TABLE 13
Co-primary Endpoints for Bio-IR Population

| | | RISA | RISA | Treatment Difference | |
|---|---|---|---|---|---|
| | PBO | 600 mg IV | 1200 mg IV | 95% CI | |
| | (N = 97) | (N = 194) | (N = 197) | RISA 600 mg | RISA 1200 mg |
| Endpoints | n (%) | n (%) | n(%) | IV - PBO | IV - PBO |
| The achievement of | 25 (25.8%) | 83 (42.6%) | 73 (37.1%) | 16.8% | 11.2% |
| clinical remission | | | | [5.6%, 27.9%] | [0.2%, 22.3%] |
| (CDAI) at Week 12 | | | | | |
| The achievement of | 11 (11.4%) | 64 (33.0%) | 47 (23.7%) | 21.6% | 12.3% |
| endoscopic response | | | | [12.5%, 30.8%] | [3.6%, 21%] |
| at Week 12 | | | | | |
| The achievement of | 22 (22.7%) | 78 (40.2%) | 76 (38.6%) | 17.5% | 15.9% |
| clinical remission | | | | [6.7%, 28.3%] | [5.1%, 26.7%] |
| (PRO2) at Week 12 | | | | | |

TABLE 14
Co-primary Endpoints for non-Bio-IR Population

| | | RISA | RISA | Treatment Difference | |
|---|---|---|---|---|---|
| | PBO | 600 mg IV | 1200 mg IV | 95% CI | |
| | (N = 78) | (N = 142) | (N = 142) | RISA 600 mg | RISA 1200 mg |
| Endpoints | n (%) | n (%) | n (%) | IV - PBO | IV - PBO |
| The achievement | 18 (23.1%) | 69 (48.6%) | 69 (48.6%) | 25.5% | 25.5% |
| of clinical | | | | [13.0%, 37.9%] | [13.0%, 37.9%] |
| remission (CDAI) | | | | | |
| The achievement | 10 (12.8%) | 71 (50.2%) | 62 (44.0%) | 37.3% | 31.2% |
| of endoscopic | | | | [26.2%, 48.4%] | [20.1%, 42.2%] |
| response at Week | | | | | |
| The achievement | 15 (19.2%) | 68 (47.9%) | 64 (45.1%) | 28.7% | 25.8% |
| of clinical | | | | [16.7%, 40.7%] | [13.9%, 37.8%] |
| remission (PRO2) | | | | | |

Safety:

Safety data were presented for all subjects who received at least one dose of study drug. The RISA 12-Week induction treatment with both IV doses was generally safe and well tolerated. The overall safety profile was consistent with the known safety profile of RISA. No new safety risks were identified.

The overall rates of treatment emergent adverse events (AEs) were similar among treatment groups. The rates of serious AEs and AEs leading to discontinuation were numerically higher in the PBO group and related to underlying disease. The rates of AEs in Areas of Safety Interest (ASI) were generally similar across the two RISA treatment groups and PBO group with exception of serious infection rate which was numerically higher in the PBO group. No events of adjudicated anaphylactic reaction, adjudicated MACE or malignancy were reported. One active tuberculosis (TB) was reported during follow-up period in a subject with latent TB in RISA 600 mg treatment group. Two deaths occurred, both in the PBO group.

Potential Impact of the Study

Both RISA 600 mg IV and 1200 mg IV for 12 weeks induction treatment achieved significantly higher clinical remission rates and endoscopic response rates compared to the PBO. Rapid response and remission in symptom relief as early as Week 4 were observed with both doses (see FIGS. 10 and 11). The RISA induction treatment for 12 weeks was generally safe and well tolerated.

### Example 7: Results of the 12-Week Induction Period of Study M15-991 (Phase 3)

Study M15-991 was a Phase 3 study to evaluate the efficacy and safety of risankizumab (RISA) during induction therapy in subjects with moderately to severely active Crohn's disease (CD) who failed prior biologic treatment. It consisted of two periods: a 12-week, randomized, double-blind, placebo-controlled induction period (12-Week Induction Period) and the exploratory Induction Period 2 for subjects who do not achieve clinical response at Week 12. The results from the 12-Week Induction Period are shown as follows.

Demographic, Baseline Characteristics, and Subject Disposition

A total of 618 subjects were randomized in the study, where 581 subjects had baseline eligible SES-CD of [symbol] 6 ( [symbol] 4 for isolated ileal disease), and an additional 37 subjects had a lower baseline eligible SES-CD. All randomized subjects received at least one dose of study drug in the 12-Week Induction Period. The intent-to-treat population for efficacy analysis in this period (ITT1A) comprised of a total of 569 subject (191* subjects in the RISA 600 mg IV group, 191* subjects in the RISA 1200 mg IV group and 187* subjects in the PBO group). Discontinuation rates were similar among three treatment groups. *12 subjects (4 subjects in the RISA 600 mg IV group, 3 subjects in the RISA 1200 mg IV group and 5 subject in the PBO group) were excluded from the efficacy analysis from a significant non-compliance site. These subjects were included in the safety analysis.

Efficacy:

The study met the co-primary endpoints and majority of the multiplicity-controlled secondary endpoints demonstrating superiority of RISA 600 mg and RISA 1200 mg intravenous (IV) dose comparing to placebo (PBO) for both US-specific protocol and global protocol outside US.

For US-specific protocol co-primary endpoints:

For the US-specific protocol, the study met the co-primary endpoints for both RISA 600 mg and 1200 mg IV dose groups comparing to the PBO group (all p-values <0.001 for clinical remission (CDAI) and all p-values <0.001 for endoscopic response). The multiplicity-controlled secondary endpoints that met or did not meet statistical significance per statistical testing procedure were presented in Table 15(A1) and Table 15 (A2).

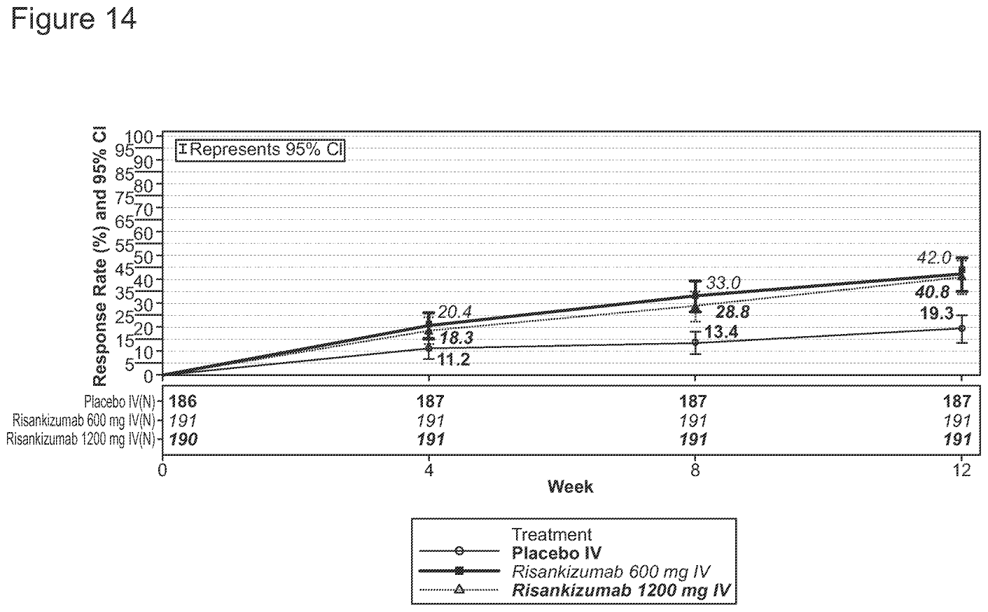

The clinical remission evaluated by Crohn's disease activity index (CDAI) rate at Week 12 was significantly higher in the RISA 600 mg and RISA 1200 mg IV induction doses (42.0% and 40.8%, respectively) compared to the PBO (19.3%) with p-value <0.001 for both dose groups (see FIG. 14).

The endoscopic response rate at Week 12 was significantly higher in the RISA 600 mg and RISA 1200 mg IV induction doses (28.8% and 34.1%, respectively) compared to the PBO (11.2%) with p-value <0.001 for both dose groups.

For global protocol outside US co-primary endpoints:

For the global protocol outside US, the study met the co-primary endpoints for both RISA 600 mg and 1200 mg IV dose groups comparing to the PBO group (all p-values <=0.001 for clinical remission (PRO2) and all p-values <0.001 for endoscopic response). The multiplicity-controlled secondary endpoints that met or did not meet statistical significance per statistical testing procedure were presented in Table 15(B1) and Table 15(B2).

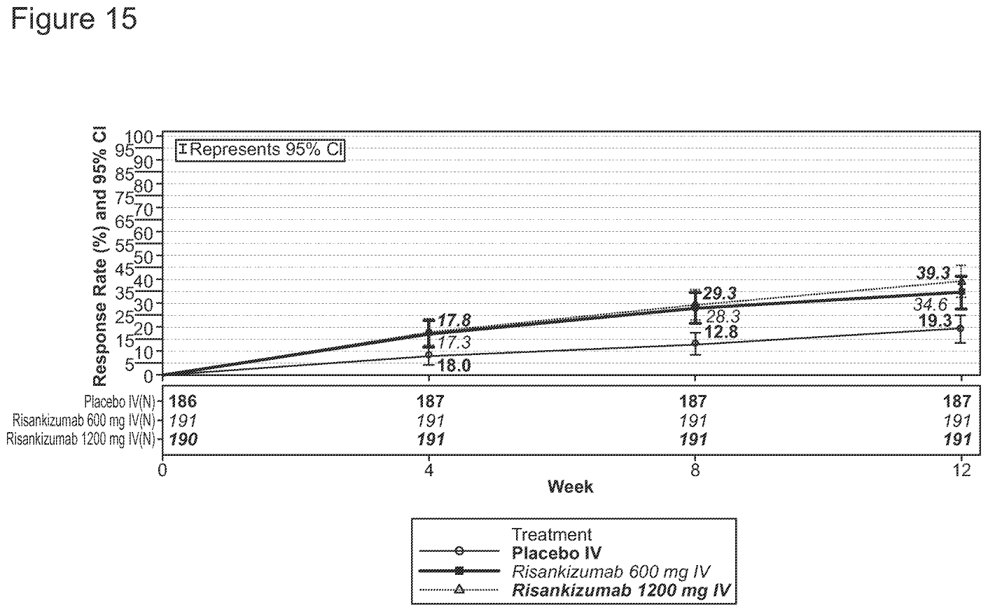

The clinical remission (PRO2*) rate at Week 12 was significantly higher in the RISA 600 mg and RISA 1200 mg IV induction doses (34.6% and 39.3%, respectively) compared to the PBO (19.3%) with p-value <=0.001 for both dose groups (see FIG. 15). *PRO2: Patient Reported Outcome related to stool frequency and abdominal pain which are components of CDAI.

The endoscopic response rate at Week 12 was significantly higher in the RISA 600 mg and RISA 1200 mg IV induction dose (28.8% and 34.1%, respectively) compared to the PBO (11.2%) with p-value <0.001 for both dose groups.

Clinical response and remission were seen as early as Week 4. Clinical remission rates over time were shown in FIG. 14 and FIG. 15.

TABLE 15
Efficacy Results for Co-primary and Secondary Efficacy Endpoints (ITT1A1)
A1) Efficacy results for US-specific protocol following first interim database lock

| | | | | RISA | | RISA | | | | | | | |
|---|---|---|---|---|---|---|---|---|---|---|---|---|---|
| | | PBO | | 600 mg IV | | 1200 mg IV | | Adjusted treatment Difference | | | | | |
| | | (N = 187) | | (N = 191) | | (N = 191) | | 95% CI³ | | | | p-Value³ | |
| | | % (n)⁴ or | | % (n)⁴ or | | % (n)⁴ or | | Δ (RISA | | Δ (RISA | | RISA | RISA |
| | | LSMEAN | | LSMEAN | | LSMEAN | | 600 mg | | 1200 mg | | 600 mg | 1200 mg |
| | Endpoints² | (SE) | | (SE) | | (SE) | | IV-PBO) | | IV-PBO) | | IV vs. PBO | IV vs. PBO |
| Primary | The achievement of CDAI | 19.3% | (36) | 42.0% | (80) | 40.8% | (78) | 22.5% | [13.6%, 31.4%] | 21.6% | [12.7%, 30.6%] | <0.001^{S} | <0.001^{S} |
| | clinical remission at Week 12 | | | | | | | | | | | | |
| | The achievement of | 11.2% | (21) | 28.8% | (55) | 34.1% | (65) | 17.8% | [10.0%, 25.5%] | 23.2% | [15.2%, 31.1%] | <0.001^{S} | <0.001^{S} |
| | endoscopic response at | | | | | | | | | | | | |
| | Week 12 | | | | | | | | | | | | |
| Secondary | 1. The achievement of clinical | 19.3% | (36) | 34.6% | (66) | 39.3% | (75) | 15.2% | [6.4%, 23.9%] | 19.9% | [11.0%, 28.8%] | 0.001^{S} | <0.001^{S} |
| Endpoints: | remission at Week 12 | | | | | | | | | | | | |
| Sequential | 2. The achievement of CDAI | 21.4% | (40) | 35.6% | (68) | 32.5% | (62) | 14.1% | [5.2%, 23.0%] | 11.3% | [2.5%, 20.0%] | 0.002^{S} | 0.012^{S} |
| | clinical response at Week 4 | | | | | | | | | | | | |
| | 3. The achievement of CDAI | 31.6% | (59) | 60.6% | (116) | 61.3% | (117) | 28.9% | [19.3%, 38.5%] | 29.6% | [20.0%, 39.1%] | <0.001^{S} | <0.001^{S} |
| | clinical response at Week 12 | | | | | | | | | | | | |
| | 4. Change from baseline in | N = 143 | | N = 166 | | N = 172 | | 2.8 | [0.4, 5.1] | 3.1 | [0.8, 5.4] | 0.020^{S} | 0.009^{S} |
| | FACIT-Fatigue at Week 12 | 7.7 (0.87) | | 10.5 (0.82) | | 10.8 (0.82) | | | | | | | |
| | 5. The achievement of clinical | 11.2% | (21) | 20.4% | (39) | 18.3% | (35) | 9.0% | [1.7%, 16.3%] | 7.2% | [0.1%, 14.2%] | 0.015^{S} | 0.046^{NS} |
| | remission (CDAI) at Week 4 | | | | | | | | | | | | |
| | 6. The achievement of CDAI | 5.3% | (10) | 21.0% | (40) | 22.5% | (43) | 15.5% | [8.9%, 22.1%] | 17.3% | [10.7%, 23.9%] | <0.001^{S} | <0.001^{NS} |
| | clinical response and endoscopic | | | | | | | | | | | | |
| | response at Week 12 | | | | | | | | | | | | |
| | 7. The achievement of SF | 28.3% | (53) | 46.1% | (88) | 48.2% | (92) | 17.4% | [7.9%, 26.9%] | 19.8% | [10.3%, 29.3%] | <0.001^{S} | <0.001^{NS} |
| | remission at Week 12 | | | | | | | | | | | | |
| | 8. The achievement of AP | 36.4% | (68) | 58.1% | (111) | 58.6% | (112) | 21.9% | [12.1%, 31.6%] | 22.3% | [12.5%, 32.0%] | <0.001^{S} | <0.001^{NS} |
| | remission at Week 12 | | | | | | | | | | | | |
| Secondary | The achievement of endoscopic | 4.3% | (8) | 19.4% | (37) | 20.4% | (39) | 15.1% | [8.9%, 21.3%] | 16.2% | [10.0%, 22.4%] | <0.001^{S} | <0.001^{NS} |
| Endpoints: | remission at Week 12 | | | | | | | | | | | | |
| Holm | The achievement of enhanced | 31.6% | (59) | 44.0% | (84) | 38.7% | (74) | 12.4% | [2.7%, 22.1%] | 7.2% | [−2.4%, 16.7%] | 0.012^{NS} | 0.141^{nS} |
| Procedure | clinical response at Week 4 | | | | | | | | | | | | |
| | The achievement of ulcer-free | N = 186 | | N = 190 | | N = 189 | | 9.6% | [3.9%, 15.3%] | 11.2% | [5.4%, 17.1%] | 0.001^{S} | <0.001^{NS} |
| | endoscopy at Week 12 | 4.3% (8) | | 13.9% (26) | | 15.3% (29) | | | | | | | |
| | The achievement of enhanced | 39.1% | (73) | 61.8% | (118) | 58.6% | (112) | 22.9% | [13.1%, 32.6%] | 19.6% | [9.8%, 29.4%] | <0.001^{S} | <0.001^{NS} |
| | clinical response at Week 12 | | | | | | | | | | | | |
| | The achievement of resolution | N = 97 | | N = 100 | | N = 97 | | 5.4% | [−7.0%, 17.8%] | 14.4% | [1.6%, 27.2%] | 0.392^{NS} | 0.027^{NS} |
| | of EIMs at Week 12, in subjects | 23.7% (23) | | 29.4% (29) | | 38.1% (37) | | | | | | | |
| | with any EIMs at baseline | | | | | | | | | | | | |
| | Occurrence of CD-related | 11.2% | (21) | 3.1% | (6) | 2.1% | (4) | −8.1% | [−13.2%, −2.9%] | −9.1% | [−14.1%, −4.2%] | 0.002^{S} | <0.001^{NS} |
| | hospitalization through Week 12 | | | | | | | | | | | | |

| | | | | | | | | | |
|---|---|---|---|---|---|---|---|---|---|
| The achievement of no draining | N = 14 | N = 14 | N = 16 | −7.1% | [−29.9%, 15.6%] | 35.7% | [5.1%, 66.3%] | 1.000^{NS} | 0.058^{NS} |
| fistulas at Week 12 in subjects | 14.3% (2) | 7.1% (1) | 50.0% (8) | | | | | | |
| with draining fistulas at baseline | | | | | | | | | |

¹1TT1A: randomized subjects who received at least one dose of study drug in the 12-Week Induction Period who had baseline eligible SES-CD of ≥6 (≥4 for isolated ileal disease).
²Definition of the endpoints are included in Example 4.
³Adjusted treatment difference, 95% CI and p-values for comparison of binary endpoints between RISA and PBO were calculated using Cochran-Mantel-Haenszel (CMH) test adjusted for randomization stratification
factors (the number of prior biologies failed (<=1, >1) and baseline corticosteroid use (yes or no)); 95% CI and p-values for comparison of continuous endpoints between RISA and PBO were calculated using
MMRM (mixed effect model repeat measurement) with baseline, treatment, visit, treatment by visit interaction and stratification factors in the model.
⁴The % (n) represents the synthesized results from multiple imputation.
^{S, NS}The endpoint achieved or did not achieve, statistical significance based on the pre-specified graphical testing procedure for the US-specific protocol.
A2) Efficacy results for US-specific protocol following second interim database lock

| | | | | RISA | | RISA | | | |
|---|---|---|---|---|---|---|---|---|---|
| | | PBO | | 600 mg IV | | 1200 mg IV | | | |
| | | (N = 187) | | (N = 191) | | (N = 191) | | p-Value² | |
| | | % (n)¹ or | | % (n)¹ or | | % (n)¹ or | | RISA | RISA |
| | | LSMEAN | | LSMEAN | | LSMEAN | | 600 mg | 1200 mg |
| Endpoints | | (SE) | | (SE) | | (SE) | | IV vs. PBO | IV vs. PBO |
| 1 | Clinical remission (PRO2) at Week 12 | 19.3% | (36) | 34.6% | (66) | 39.3% | (75) | 0.001 | <0.001 |
| 2 | CDAI clinical response at Week 4 | 20.9% | (39) | 36.6% | (70) | 32.5% | (62) | 0.001 | 0.008 |
| 3 | CDAI clinical response at Week 12 | 30.0% | (56) | 60.0% | (115) | 61.3% | (117) | <0.001 | <0.001 |
| 4 | Change from baseline in FACIT-Fatigue at Week 12 | 7.7 | (0.87) | 10.5 | (0.82) | 10.8 | (0.82) | 0.020 | 0.009 |
| 5 | Clinical remission (CDAI) at Week 4 | 11.2% | (21) | 20.9% | (40) | 19.4% | (37) | 0.010 | 0.023 |
| 6 | CDAI clinical response and endoscopic response at | 5.3% | (10) | 21.0% | (40) | 23.0% | (44) | <0.001 | <0.001 |
| | Week 12 | | | | | | | | |
| 7 | SF remission at Week 12 | 28.3% | (53) | 46.1% | (88) | 48.7% | (93) | <0.001 | <0.001 |
| 8 | AP remission at Week 12 | 36.4% | (68) | 58.1% | (111) | 58.6% | (112) | <0.001 | <0.001 |
| Holm | Endoscopic remission at Week 12 | 4.3% | (8) | 19.4% | (37) | 20.4% | (39) | <0.001 | <0.001 |
| Procedure | Enhanced clinical response (PRO2) at Week 4 | 31.6% | (59) | 44.0% | (84) | 38.7% | (74) | 0.006 | 0.142^{NS} |
| | Ulcer-free endoscopy at Week 12 | N = 186 | | N = 190 | | N = 189 | | 0.001 | <0.001 |
| | | 4.3% (8) | | 13.9% (26) | | 15.3% (29) | | | |
| | Enhanced clinical response (PRO2) at Week 12 | 39.1% | (73) | 61.8% | (118) | 58.6% | (112) | <0.001 | <0.001 |
| | Resolution of EIMs at Week 12, in subjects with any | N = 97 | | N = 100 | | N = 97 | | 0.377^{NS} | 0.027^{NS} |
| | EIMs at baseline | 23.7% (23) | | 29.4% (29) | | 38.1% (37) | | | |
| | CD-related hospitalization through Week 12 | 11.2% | (21) | 3.1% | (6) | 2.1% | (4) | 0.002 | <0.001 |

| | | | | | |
|---|---|---|---|---|---|
| No draining fistulas at Week 12 in subjects with | N = 14 | N = 14 | N = 16 | 1.000^{NS} | 0.058^{NS} |
| draining fistulas at baseline | 14.3% (2) | 7.1% (1) | 50.0% (8) | | |

¹The % (n) represents the synthesized results from multiple imputation.
²Adjusted treatment difference, 95% CI and p-values for comparison of binary endpoints between RISA and placebo were calculated using Cochran-Mantel-Haenszel (CMH) test adjusted for randomization stratification
factors (the number of prior biologies failed and baseline corticosteroid use); 95% CI and p-values for comparison of continuous endpoints between RISA and placebo were calculated using MMRM (mixed effect model
repeat measurement) with baseline, treatment, visit, treatment by visit interaction and stratification factors in the model.
^{NS}Not statistically significant based on adjusted p-value from the graphical testing procedure
ITT1A: includes randomized subjects who received at least one dose of study drug who had baseline eligible SES-CD of ≥6 (≥4 for isolated ileal disease), excluding subjects from the non-compliant site
(Investigator ID 527969).
B1) Efficacy results for global protocol outside US following first interim database lock

| | | | | RISA | | RISA | | | | | | | |
|---|---|---|---|---|---|---|---|---|---|---|---|---|---|
| | | PBO | | 600 mg IV | | 1200 mg IV | | Adjusted treatment Difference | | | | | |
| | | (N = 187) | | (N = 191) | | (N = 191) | | 95% CI³ | | | | p-Value³ | |
| | | % (n)⁴ or | | % (n)⁴ or | | % (n)⁴ or | | Δ (RISA | | Δ (RISA | | RISA | RISA |
| | | LSMEAN | | LSMEAN | | LSMEAN | | 600 mg | | 1200 mg | | 600 mg | 1200 mg |
| | Endpoints² | (SE) | | (SE) | | (SE) | | IV-PBO) | | IV-PBO) | | IV vs. PBO | IV vs. PBO |
| | The achievement of | 19.3% | (36) | 34.6% | (66) | 39.3% | (75) | 15.2% | [6.4%, 23.9%] | 19.9% | [11.0%, 28.8%] | 0.001^{S} | <0.001^{S} |
| | clinical remission (PRO2) | | | | | | | | | | | | |
| | The achievement of | 11.2% | (21) | 28.8% | (55) | 34.1% | (65) | 17.8% | [10.0%, 25.5%] | 23.2% | [15.2%, 31.1%] | <0.001^{S} | <0.001^{S} |
| | endoscopic response | | | | | | | | | | | | |
| | at Week 12 | | | | | | | | | | | | |
| Secondary | 1. The achievement of clinical | 19.3% | (36) | 42.0% | (80) | 40.8% | (78) | 22.5% | [13.6%, 31.4%] | 21.6% | [12.7%, 30.6%] | <0.001^{S} | <0.001^{S} |
| Endpoints: | remission (CDAI) at Week 12 | | | | | | | | | | | | |
| Sequential | 2. The achievement of CDAI | 21.4% | (40) | 35.6% | (68) | 32.5% | (62) | 14.1% | [5.2%, 23.0%] | 11.3% | [2.5%, 20.0%] | 0.002^{S} | 0.012^{S} |
| | clinical response at Week 4 | | | | | | | | | | | | |
| | 3. The achievement of clinical | 8.0% | (15) | 17.3% | (33) | 17.8% | (34) | 9.2% | [2.6%, 15.7%] | 9.8% | [3.3%, 16.3%] | 0.006^{S} | 0.003^{S} |
| | remission at Week 4 | | | | | | | | | | | | |
| | 4. The achievement of CDAI | 31.6% | (59) | 60.6% | (116) | 61.3% | (117) | 28.9% | [19.3%, 38.5%] | 29.6% | [20.0%, 39.1%] | <0.001^{S} | <0.001^{S} |
| | clinical response at Week 12 | | | | | | | | | | | | |
| | 5. Change from baseline in | N = 143 | | N = 166 | | N = 172 | | 2.8 | [0.4, 5.1] | 3.1 | [0.8, 5.4] | 0.020^{S} | 0.009^{S} |
| | FACIT-Fatigue at Week 12 | 7.7 (0.87) | | 10.5 (0.82) | | 10.8 (0.82) | | | | | | | |
| | 6. Change from baseline in IBDQ | N = 143 | | N = 166 | | N = 172 | | 12.1 | [4.6, 19.6] | 15.3 | [7.8, 22.7] | 0.002^{S} | <0.001^{S} |
| | total score at Week 12 | 27.0 (2.79) | | 39.0 (2.63) | | 42.3 (2.62) | | | | | | | |
| Secondary | The achievement of enhanced | 7.0% | (13) | 21.0% | (40) | 24.1% | (46) | 14.0% | [7.2%, 20.8%] | 17.4% | [10.4%, 24.3%] | <0.001^{S} | <0.001^{S} |
| Endpoints: | clinical response and endoscopic | | | | | | | | | | | | |
| Holm | response at Week 12 | | | | | | | | | | | | |
| Procedure | The achievement of endoscopic | 4.3% | (8) | 19.4% | (37) | 20.4% | (39) | 15.1% | [8.9%, 21.3%] | 16.2% | [10.0%, 22.4%] | <0.001^{S} | <0.001^{S} |
| | remission at Week 12 | | | | | | | | | | | | |
| | The achievement of enhanced | 31.6% | (59) | 44.0% | (84) | 38.7% | (74) | 12.4% | [2.7%, 22.1%] | 7.2% | [−2.4%, 16.7%] | 0.012^{NS} | 0.141^{NS} |
| | clinical response at Week 4 | | | | | | | | | | | | |
| | The achievement of ulcer-free | N = 186 | | N = 190 | | N = 189 | | 9.6% | [3.9%, 15.3%] | 11.2% | [5.4%, 17.1%] | 0.001^{S} | <0.001^{S} |
| | endoscopy at Week 12 | 4.3% (8) | | 13.9% (26) | | 15.3% (29) | | | | | | | |
| | The achievement of enhanced | 39.1% | (73) | 61.8% | (118) | 58.6% | (112) | 22.9% | [13.1%, 32.6%] | 19.6% | [9.8%, 29.4%] | <0.001^{S} | <0.001^{S} |
| | clinical response at Week 12 | | | | | | | | | | | | |
| | The achievement of resolution | N = 97 | | N = 100 | | N = 97 | | 5.4% | [−7.0%, 17.8%] | 14.4% | [1.6%, 27.2%] | 0.392^{NS} | 0.027^{NS} |
| | of EIMs at Week 12, in subjects | 23.7% (23) | | 29.4% (29) | | 38.1% (37) | | | | | | | |
| | with any EIMs at baseline | | | | | | | | | | | | |
| | Occurrence of CD-related | 11.2% | (21) | 3.1% | (6) | 2.1% | (4) | −8.1% | [−13.2%, −2.9%] | −9.1% | [−14.1%, −4.2%] | 0.002^{S} | <0.001^{S} |
| | hospitalization through Week 12 | | | | | | | | | | | | |

| | | | | | | | | | |
|---|---|---|---|---|---|---|---|---|---|
| The achievement of no draining | N = 14 | N = 14 | N = 16 | −7.1% | [−29.9%, 15.6%] | 35.7% | [5.1%, 66.3%] | 1.000^{NS} | 0.058^{NS} |
| fistulas at Week 12 in subjects | 14.3% (2) | 7.1% (1) | 50.0% (8) | | | | | | |
| with draining fistulas at baseline | | | | | | | | | |
| Change from baseline in WPAI- | N = 68 | N = 73 | N = 86 | −7.3 | [−16.4, 1.7] | −8.8 | [−17.6, −0.1] | 0.113^{NS} | 0.048^{NS} |
| CD Overall Work Impairment at | −12.2 (3.37) | −19.5 (3.27) | −21.0 (3.01) | | | | | | |
| Week 12 | | | | | | | | | |
| Change from baseline in SF-36 | N = 141 | N = 165 | N = 172 | 2.2 | [0.5, 3.9] | 2.8 | [1.1, 4.4] | 0.009^{NS} | 0.001^{S} |
| Physical Component Summary | 5.2 (0.62) | 7.4 (0.58) | 8.0 (0.58) | | | | | | |
| score at Week 12 | | | | | | | | | |

¹ITT1A: randomized subjects who received at least one dose of study drug in the 12-Week Induction Period who had baseline eligible SES-CD of ≥6 (≥4 for isolated ileal disease).
²Definition of the endpoints are included in Example 4.
³Adjusted treatment difference, 95% CI and p-values for comparison of binary endpoints between RISA and PBO were calculated using Cochran-Mantel-Haenszel (CMH) test adjusted for randomization stratification
factors (the number of prior biologies failed (<=1, >1) and baseline corticosteroid use (yes or no)); 95% CI and p-values for comparison of continuous endpoints between RISA and PBO were calculated using
MMRM (mixed effect model repeat measurement) with baseline, treatment, visit, treatment by visit interaction and stratification factors in the model.
⁴The % (n) represents the synthesized results from multiple imputation.
^{S, NS}The endpoint achieved, did not achieve, statistical significance based on the pre-specified graphical testing procedure for the global protocol outside US.

| B2) Efficacy results for global protocol outside US following second interim database lock | | | | |
|---|---|---|---|---|
| | | RISA | RISA | |
| | PBO | 600 mg IV | 1200 mg IV | |
| | (N = 187) | (N = 191) | (N = 191) | p-Value² |

| | | % (n)¹ or | | % (n)¹ or | | % (n)¹ or | | RISA | RISA |
|---|---|---|---|---|---|---|---|---|---|
| | | LSMEAN | | LSMEAN | | LSMEAN | | 600 mg | 1200 mg |
| Endpoints | | (SE) | | (SE) | | (SE) | | IV vs. PBO | IV vs. PBO |
| 1 | Clinical remission | 19.8% | (37) | 42.5% | (81) | 40.3% | (77) | <0.001 | <0.001 |
| | (CDAI) at Week 12 | | | | | | | | |
| 2 | CDAI clinical response | 20.9% | (39) | 36.6% | (70) | 32.5% | (62) | 0.001 | 0.008 |
| | at Week 4 | | | | | | | | |
| 3 | Clinical remission (PRO2) | 8.0% | (15) | 17.3% | (33) | 18.3% | (35) | 0.006 | 0.002 |
| | at Week 4 | | | | | | | | |
| 4 | CDAI clinical response | 30.0% | (56) | 60.0% | (115) | 61.3% | (117) | <0.001 | <0.001 |
| | at Week 12 | | | | | | | | |
| 5 | Change from baseline in | 7.7 | (0.87) | 10.5 | (0.82) | 10.8 | (0.82) | 0.020 | 0.009 |
| | FACIT-Fatigue at Week 12 | | | | | | | | |
| 6 | Change from baseline in | 27.0 | (2.79) | 39.0 | (2.63) | 42.3 | (2.62) | 0.002 | <0.001 |
| | IBDQ total score at Week 12 | | | | | | | | |
| Holm | Enhanced clinical response | 7.0% | (13) | 21.0% | (40) | 24.1% | (46) | <0.001 | <0.001 |
| Procedure | (PRO2) and endoscopic | | | | | | | | |
| | response at Week 12 | | | | | | | | |
| | Endoscopic remission | 4.3% | (8) | 19.4% | (37) | 20.4% | (39) | <0.001 | <0.001 |
| | at Week 12 | | | | | | | | |
| | Enhanced clinical | 31.6% | (59) | 45.0% | (86) | 38.7% | (74) | 0.006^{NS} | 0.141^{NS} |
| | response (PRO2) at Week 4 | | | | | | | | |
| | Ulcer-free endoscopy | N = 186 | | N = 190 | | N = 189 | | 0.001 | <0.001 |
| | at Week 12 | 4.3% (8) | | 13.9% (26) | | 15.3% (29) | | | |
| | Enhanced clinical response | 39.1% | (73) | 61.8% | (118) | 58.6% | (112) | <0.001 | <0.001 |
| | (PRO2) at Week 12 | | | | | | | | |
| | Resolution of EIMs at Week | N = 97 | | N = 100 | | N = 97 | | 0.377^{NS} | 0.027^{NS} |
| | 12, in subjects with | 23.7% (23) | | 29.4% (29) | | 38.1% (37) | | | |
| | any EIMs at baseline | | | | | | | | |
| | CD-related hospitalization | 11.2% | (21) | 3.1% | (6) | 2.1% | (4) | 0.002 | <0.001 |
| | through Week 12 | | | | | | | | |
| | No draining fistulas at | N = 14 | | N = 14 | | N = 16 | | 1.000^{NS} | 0.058^{NS} |
| | Week 12 in subjects with | 14.3% (2) | | 7.1% (1) | | 50.0% (8) | | | |
| | draining fistulas at baseline | | | | | | | | |
| | Change from baseline in | −12.217 | (3.3654) | −19.542 | (3.2721) | −21.034 | (3.0052) | 0.113^{NS} | 0.050^{NS} |
| | WPAI-CD Overall Work | | | | | | | | |
| | Impairment at Week 12 | | | | | | | | |
| | Change from baseline in | 5.193 | (0.6195) | 7.389 | (0.5806) | 7.951 | (0.5767) | 0.009^{NS} | 0.001 |
| | SF-36 Physical Component | | | | | | | | |
| | Summary score at Week 12 | | | | | | | | |

¹The % (n) represents the synthesized results from multiple imputation.
Adjusted treatment difference, 95% CI and p-values for comparison of binary endpoints between RISA and placebo were calculated using Cochran-Mantel-Haenszel (CMH) test adjusted for randomization stratification
factors (the number of prior biologies failed and baseline corticosteroid use); 95% CI and p-values for comparison of continuous endpoints between RISA and placebo were calculated using MMRM(mixed effect model
repeat measurement) with baseline, treatment, visit, treatment by visit interaction and stratification factors in the model.
ITT1A: includes randomized subjects who received at least one dose of study drug who had baseline eligible SES-CD of ≥6 (≥4 for isolated ileal disease), excluding subjects from the non-compliant site
(Investigator ID 527969).
^{NS}Not statistically significant based on adjusted p-value from the graphical testing procedure

Safety:

Safety data were presented for all subjects who received at least one dose of study drug. The RISA 12-Week induction treatment with both 600 mg and 1200 mg IV doses was generally safe and well tolerated. No new safety risks were identified and the overall safety profile was consistent with the known safety profile of RISA.

The overall incidence of treatment emergent adverse events (AEs) was numerically higher in the PBO group in the 12-Week Induction Period. The rates of serious AEs, severe AEs, and AEs leading to discontinuation were numerically higher in the PBO group and related to underlying disease in the 12-Week Induction Period. The incidences of AEs in areas of safety interest (ASI) were similar across treatment groups with exception of serious infections, which were numerically higher in the PBO group (2.4%), compared to 0.5% in RISA 600 mg and 1.0% in RISA 1200 mg. No events of tuberculosis (TB), adjudicated anaphylactic reaction, adjudicated MACE, or malignancy were reported. One death was reported in a subject with malignancy in the RISA 1200 mg group who had a lower baseline SES-CD.

Potential Impact of the Study

Both RISA 600 mg IV and 1200 mg IV for 12 weeks induction treatment achieved significantly higher clinical remission rates and endoscopic response rates compared to the PBO. Rapid response and remission in symptom relief as early as Week 4 were observed with both doses. The RISA induction treatment for 12 weeks was generally safe and well tolerated.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

### Example 8: Achievement of Steroid-Free Remission in Patients with Moderately to Severely Active Crohn's Disease During Treatment with Risankizumab

Background

Steroid-free clinical remission is an important treatment goal in CD. The efficacy of RZB at induction by baseline steroid use and steroid-free outcomes during maintenance was examined.

Methods:

In ADVANCE (NCT03105128) and MOTIVATE (NCT03104413), patients with moderately to severely active CD received intravenous (IV) RZB induction therapy or placebo (PBO) for 12 weeks. Patients with clinical response to RZB were re-randomised in a 52-week maintenance study (FORTIFY; NCT03105102) to subcutaneous (SC) RZB or PBO (withdrawal). Patients receiving steroids at baseline of the induction studies must have maintained stable doses for the 12-week study duration. A forced steroid taper was initiated at week 0 of maintenance for patients receiving steroids at induction baseline. Patients losing clinical response (per investigator assessment) after initiation of taper could have their steroid dose increased up to that used at induction baseline. This analysis included patients who received RZB 600 mg IV in ADVANCE or MOTIVATE and patients who received RZB 360 mg SC in FORTIFY. Endpoints reported included clinical remission (CD Activity Index [CDAI] or stool frequency/abdominal pain score [SF/APS] criteria) at week 12 of induction by baseline steroid use, steroid-free clinical remission (CDAI or SF/APS), steroid-free endoscopic response, and steroid-free endoscopic remission at week 52 of maintenance. Steroid discontinuation rates over 52 weeks of maintenance were also assessed.

Results:

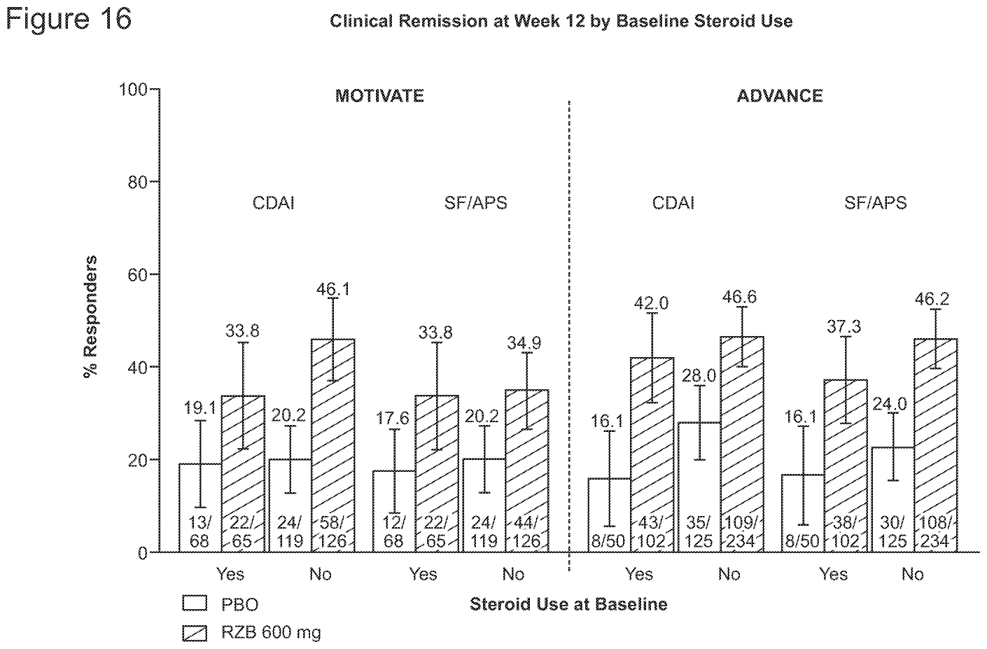

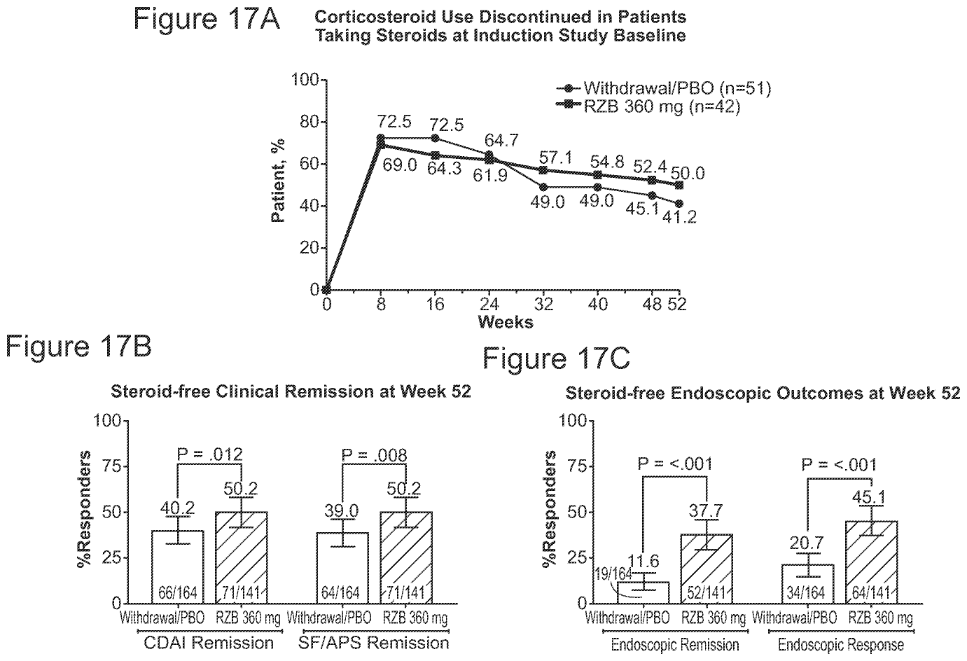

At week 12 of induction, numerically greater proportions of patients receiving RZB 600 mg IV in ADVANCE or MOTIVATE achieved clinical remission vs PBO, regardless of baseline steroid use (FIG. 16). Clinical remission rates (CDAI or SF/APS) at week 12 were similar for patients using steroids vs those who were not (33.8%-42.0% vs 34.9%-46.6%). Of patients using steroids during the induction studies, rates of steroid use decreased over time in FORTIFY, with a greater proportion of patients receiving RZB 360 mg SC discontinuing steroids at week 52 vs withdrawal/PBO (FIG. 17A). Rates of steroid-free clinical remission (P≤0.012), steroid-free endoscopic response (P<0.001), and steroid-free endoscopic remission (P<0.001) were significantly higher with RZB 360 mg SC vs PBO withdrawal at week 52 (FIGS. 17B and 17C).

Conclusion:

Steroid use at study baseline did not affect clinical remission rates at induction with IV RZB therapy. RZB SC maintenance therapy led to steroid-free clinical and endoscopic outcomes, demonstrating RZB treatment benefit in CD.

Of note, the “Steroid-free Clinical Remission at Week 52” criteria used in the data analysis in FIG. 17B is different than the “Steroid-free clinical remission” criteria on Table 10 (secondary endpoint #6). In particular, FIG. 17B's end point criteria included all study participants, regardless of whether they required steroids at the study onset (baseline), whereas Table 10's endpoint included only those subjects requiring steroid treatment at the study onset (baseline). Also, FIG. 17B's end point criteria did not require a minimum 90 days off steroids—only that the subject achieved clinical remission and was not receiving steroids at wk 52, whereas Table 10's endpoint criteria required both a minimum 90 days steroid-free and remission at wk 52.

### Example 9: UC Induction Study M16-067

A multicenter, randomized, double-blind, placebo-controlled induction study (M16-067) was carried out to evaluate the efficacy and safety of risankizumab in subjects with moderately to severely active ulcerative colitis.

Objective:

The study M16-067 comprised two sub-studies: the objective of Sub-Study 4 (Phase 2b induction) was to characterize the efficacy, safety, and pharmacokinetics of risankizumab as induction treatment in subjects with moderately to severely active UC and to identify the appropriate induction dose of risankizumab for further evaluation in Sub-Study 5 (Phase 3 induction). Sub-Study 4 had closed enrollment and all subjects in the double-blind, placebo-controlled portion of the study had completed induction.

The objective of Sub-Study 5 (Phase 3 induction) was to evaluate the efficacy and safety of risankizumab compared to placebo in inducing clinical remission in subjects with moderately to severely active UC.

Study Sites:

There were approximately 400 sites worldwide.

Study Population:

Males and females [symbol] 18 and [symbol] 80 years of age, or minimum age of adult consent according to local regulations, or aged 16 to <18 year of age who met the definition of Tanner stage 5 development where locally permitted with a diagnosis of moderately to severely active UC, defined as Adapted Mayo score of 5-9 points (using the Mayo scoring system, excluding Physician's Global Assessment) with an endoscopic subscore of 2 or 3 on screening endoscopy, confirmed by central review.

Sub-Study 4 enrolled subjects who have had an inadequate response (IR) to prior biologic therapy (bio-IR). Sub-Study 5 enrolled subjects who have had an inadequate response (IR) to prior biologic therapy (bio-IR) and subjects who have not had an inadequate response to prior biologic therapy (non-bio-IR). The bio-IR enrollment was approximately 541 subjects and the non-bio-IR enrollment was approximately 425 subjects.

The bio-IR population was defined as subjects with documented intolerance or inadequate response to one or more of the approved biologics for UC (infliximab, adalimumab, golimumab, and/or vedolizumab) or tofacitinib.

The non-bio-IR population included subjects who had an inadequate response or intolerance to conventional therapy. Conventional therapy was defined as one or more of the following: aminosalicylates, oral locally acting steroids (e.g., budesonide, beclomethosone), systemic corticosteroids (prednisone or equivalent), or immunomodulators. This population also included subjects who have received biologic therapy or tofacitinib in the past but stopped therapy based on reasons other than inadequate response or intolerance (e.g., change in reimbursement coverage, well-controlled disease).

Number of Subjects Enrolled:

Approximately 1578 subjects in total: 240 subjects in Sub-Study 4, Phase 2b double-blind, placebo-controlled induction; approximately 372 subjects in Sub-Study 4 enrolled during open-label (OL) dose selection period, and approximately 966 subjects in Sub-Study 5 Phase 3 induction.

Methodology:

This Phase 2b/3 study had an operationally seamless design and comprised two sub-studies. The purpose of this design was to seamlessly transition from the Phase 2b induction study to the Phase 3 induction study without enrollment pause. Sub-Study 4 was designed as a Phase 2b dose finding study to evaluate the efficacy, safety, and PK of risankizumab as induction treatment to identify the appropriate induction dose of risankizumab for further evaluation in Sub-Study 5. Sub-Study 5 was a Phase 3 induction study to evaluate the efficacy and safety of risankizumab versus placebo.

Sub-Study 4 (Phase 2b Induction):

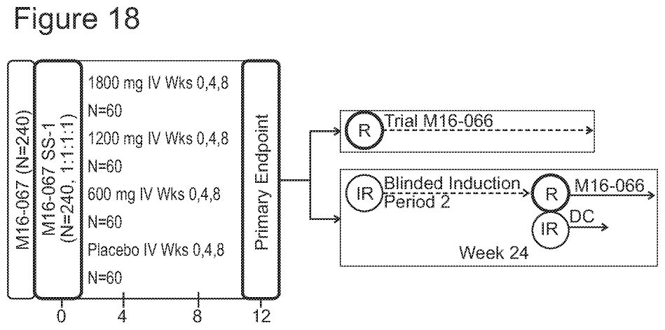

Induction Period 1 (FIG. 18):

Subjects (n=240) who met all of the inclusion criteria and none of the exclusion criteria were randomized into the study in a 1:1:1:1 ratio to one of the following treatment groups:

Group 1: Risankizumab 1800 mg IV Weeks 0, 4, 8 (n=60);

Group 2: Risankizumab 1200 mg IV Weeks, 0, 4, 8 (n=60);

Group 3: Risankizumab 600 mg IV Weeks 0, 4, 8 (n=60); and

Group 4: Placebo IV Weeks 0, 4, 8 (n=60).

The randomization at baseline was stratified by baseline steroid use (yes vs no) and baseline Adapted Mayo score (≤7 vs >7). Endoscopy and evaluation of clinical response and remission occurred at Week 12.

Subjects in Sub-Study 4 who achieved clinical response per Adapted Mayo score (locally read Mayo endoscopic subscore) after completion of the 12-week Induction Period 1 were eligible to be enrolled into maintenance Study M16-066. Clinical response was defined as a decrease from baseline in the Adapted Mayo score [symbol] 2 points and [symbol] 30% from baseline, PLUS a decrease in rectal bleeding subscore (RBS) [symbol] 1 or an absolute RBS [symbol] 1.

Subjects who did not achieve clinical response at Week 12 were eligible to receive blinded risankizumab treatment in Induction Period 2 as specified below. Subjects were not eligible to enter Induction Period 2 until the Week 12 endoscopy has been completed.

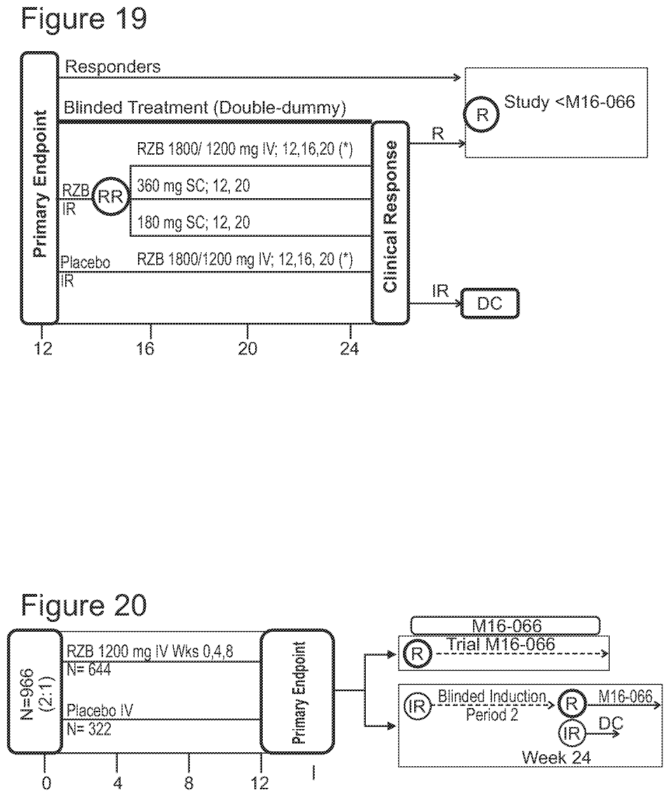

Induction Period 2 (FIG. 19):

At Week 12, subjects who did not achieve clinical response were randomized by Interactive Response Technologies (IRT) to Induction Period 2, a double-blind, double-dummy 12-week treatment period to evaluate reinduction with risankizumab versus starting maintenance dosing on clinical response status.

Subjects who received IV risankizumab induction were randomized 1:1:1 to:

Group 1: 1800 mg IV risankizumab Weeks 12, 16, 20;

Group 2: 360 mg SC risankizumab Weeks 12, 20; and

Group 3: 180 mg SC risankizumab Weeks 12, 20.

Subjects who received IV placebo induction treatment received:

Group 4: 1800 mg IV risankizumab Weeks 12, 16, 20.

Subjects randomized in groups 1 and 4 received placebo SC and subjects randomized in Groups 2 and 3 received placebo IV, in order to keep the blind. The IV risankizumab dose or matching IV placebo was given at Weeks 12, 16, and 20. The SC risankizumab dose or matching SC placebo was given at Weeks 12, and 20. At Week 24, subjects who had received blinded risankizumab treatment during Induction Period 2 were reassessed and underwent a third endoscopy for evaluation of mucosal inflammation. Subjects who achieved clinical response per Adapted Mayo score (locally read Mayo endoscopic subscore) at Week 24 were eligible to enter into the maintenance Study M16-066. Subjects without clinical response at Week 24, as well as all subjects who terminated the study early (including subjects who were eligible for but did not receive blinded risankizumab therapy during Induction Period 2), were discontinued and had a follow-up call 140 days from the last dose of study drug to obtain information on any new or ongoing AEs.

Sub-Study 5 (Phase 3 Induction):

Induction Period 1 (FIG. 20):

Approximately 966 subjects who met all of the inclusion criteria and none of the exclusion criteria were randomized into the double-blind 12-week study in a 2:1 ratio to one of the following treatment groups:

Group 1: Risankizumab 1200 mg IV Weeks 0, 4, 8 (n=644); and

Group 2: Placebo IV Weeks 0, 4, 8 (n=322).

The randomization at baseline was stratified by number of prior failed biologic treatments (0, 1 vs >1), baseline steroid use (yes vs no), and baseline Adapted Mayo score ( [symbol] 7 vs >7). Endoscopy and the primary analyses occurred at Week 12. Subjects in Sub-Study 5 who achieved clinical response after completion of the 12-week Induction Period 1 were enrolled into maintenance Study M16-066. Subjects who did not achieve clinical response per Adapted Mayo score (locally read Mayo endoscopic subscore) at Week 12 may have received blinded risankizumab treatment in Induction Period 2 as specified below. Subjects were not eligible to enter Induction Period 2 until the Week 12 endoscopy was completed.

Induction Period 2 (FIG. 19):

At Week 12, subjects who did not achieve clinical response were randomized by IRT to Induction Period 2, a double-blind, double-dummy 12-week treatment period to evaluate reinduction with risankizumab versus starting maintenance dosing on clinical response status.

Subjects who received IV risankizumab were randomized 1:1:1 to:

Group 1: 1200 mg IV risankizumab Weeks 12, 16, and 20;

Group 2: 360 mg SC risankizumab Weeks 12, and 20; and

Group 3: 180 mg SC risankizumab Weeks 12, and 20.

Subjects who received placebo induction treatment received:

Group 4: 1200 mg IV risankizumab Weeks 12, 16, and 20.

Subjects randomized in groups 1 and 4 received placebo SC and subjects randomized in Groups 2 and 3 received placebo IV, in order to keep the blind. The IV risankizumab dose or matching IV placebo was given at Weeks 12, 16, and 20. The SC risankizumab dose or matching SC placebo was given at Weeks 12, and 20. At Week 24, subjects who receive blinded risankizumab during the Induction Period 2 were reassessed and underwent a third endoscopy for evaluation of mucosal inflammation. Subjects who achieved clinical response per Adapted Mayo score (locally read Mayo endoscopic subscore) at Week 24 may have been entered into the maintenance Study M16-066. Subjects without clinical response at Week 24, as well as all subjects who terminate the study early (including subjects who were eligible for but did not receive blinded risankizumab therapy during Induction Period 2), were discontinued and had a follow-up call 140 days from the last dose of study drug to obtain information on any new or ongoing AEs.

Study Visits:

Study visits for clinical and safety assessments were performed at Baseline, Weeks 4, 8, and 12/Premature Discontinuation (PD). For subjects entering Induction Period 2, additional study visits occurred at Weeks 16, 20, and 24/PD. All subjects were provided with a subject diary where they recorded UC related symptoms, use of anti-diarrheals, and use of medications for endoscopy preparation. Additionally, subjects completed symptom, quality of life (QoL) and work productivity questionnaires throughout the study. Clinical labs including, but not limited to, urinalysis, chemistry and hematology, high-sensitivity C-reactive protein (hs-CRP), serum risankizumab concentrations, and serum anti-drug antibody (ADA) levels were collected throughout the study. In addition, stool samples for calprotectin analysis were collected before starting bowel preparations for endoscopy.

Subjects underwent endoscopies during screening and Week 12. Subjects who enter Induction Period 2 underwent an additional endoscopy at Week 24. Endoscopies were evaluated using the Mayo endoscopic subscore and the presence or absence of friability was documented. All endoscopies were video recorded. Videos from subjects with eligible Mayo endoscopic sub-scores during Screening and all videos from subjects at Week 12 and Week 24 were sent to a central review vendor and scored as described in the central review charter. In addition, the central reader assessed the endoscopy findings using the Ulcerative Colitis Endoscopic Index of Severity (UCEIS) for additional exploratory analyses. Biopsy samples for histologic assessment were collected at each endoscopy visit. Additional biopsies to confirm diagnosis (during Screening) or to rule out dysplasia/malignancy may have been performed during the same time points as the endoscopy. For subjects who consented, optional exploratory research samples may have been taken during the study.

Subjects were discontinued from the study if they withdrew consent or if they were deemed unsuitable to continue for any reason by the Investigator.

Diagnosis and Main Criteria for Inclusion/Exclusion:

The following Inclusion/Exclusion Criteria were for subjects enrolled in either Sub-Study 4 or 2, except where specified.

Main Inclusion:

1. Males or females ≥18 and ≤80 years of age, or minimum age of adult consented according to local regulations at the Baseline Visit. In addition for Sub-Study 5 only: locally permissible, subjects 16 to <18 years of age who met the definition of Tanner Stage 5 for development at the Baseline Visit.

2. Confirmed diagnosis of UC for at least 3 months prior to Baseline. Appropriate documentation of biopsy results consistent with the diagnosis of UC or in the assessment of the Investigator, must be available.

3. Active UC with an Adapted Mayo score of 5 to 9 points and endoscopic subscore of 2 to 3 (confirmed by central review).

4. Demonstrated intolerance or inadequate response to one or more of the following categories of drugs: aminosalicylates, oral locally acting steroids, systemic steroids (prednisone or equivalent), immunomodulators, and/or biologic therapies or tofacitinib.

Demonstration of intolerance requires no minimum dose or duration of use.

Inadequate response was defined as outlined below:
Oral aminosalicylates (e.g., mesalamine, sulfasalazine, olsalazine, balsalazide):
   Signs and symptoms of persistently active disease, in the opinion of the Investigator, during a current or prior course of at least 4 weeks of treatment with 2.4 g/day mesalamine (2 g/day if controlled release), 4 g/day sulfasalazine, 1 g/day olsalazine, or 6.75 g/day balsalazide,
   Oral locally acting steroids (e.g., budesonide, beclomethasone):
      Signs and symptoms of persistently active disease, in the opinion of the Investigator, during or after a course of at least 4 weeks of treatment with 9 mg/day budesonide or 5 mg/day beclomethasone,
      OR
      Inability to taper oral budesonide to at or below 6 mg/day without recurrent active disease,
   IV or Oral systemic steroids (prednisone or equivalent):
      Signs and symptoms of persistently active disease, in the opinion of the Investigator, during or after tapering of at least one regimen consisting of a dose equivalent to prednisone ≥40 mg/day orally for 3 weeks or intravenously for 1 week,
      OR
      Inability to taper oral systemic steroids to at or below a dose equivalent to prednisone 10 mg/day without recurrent active disease,
   Immunomodulators:
      Signs and symptoms of persistently active disease, in the opinion of the Investigator, during a current or prior course of at least 90 days of treatment with one or more of the following:
         AZA: ≥2.0 mg/kg/day rounded to the nearest available tablet or half tablet formulation (≥1 mg/kg/day for subjects in Japan, Korea, Taiwan, Singapore, or China) (or a documented 6-TGN level of ≥230 pmol/8×10⁸ RBC)
         6-MP: ≥1 mg/kg/day rounded to the nearest available tablet or half tablet formulation (≥0.6 mg/kg/day for subjects in Japan, Korea, Taiwan, Singapore, or China) (or a 6-TGN level of ≥230 pmol/8×10⁸ RBC)
         MTX: ≥15 mg/week subcutaneous (SC) or intramuscular (IM)
         Note: Oral MTX use was allowed during the study, however prior or current use of oral MTX was not sufficient for inclusion into the study
         Tacrolimus: (for Japan, Taiwan and other countries in Asia with local treatment guidelines that include tacrolimus) documented trough level 5-10 ng/mL
      Biologic Therapies and tofacitinib for UC: Signs and symptoms of persistently active disease despite a history of one or more of the following:
         At least one 6-week induction regimen of infliximab (≥5 mg/kg intravenous [IV] at Weeks 0, 2, and 6),
         At least one 4-week induction regimen of adalimumab (one 160 mg SC dose at Week 0, followed by one 80 mg SC dose at Week 2 [or one 80 mg SC dose at Week 0, followed by one 40 mg SC dose at Week 2, in countries where this dosing regimen is approved]),
         At least one 4-week induction regimen of golimumab (200 mg SC at Week 0 and 100 mg SC at Week 2),
         At least one 6-week induction regimen of vedolizumab (300 mg IV at Weeks 0, 2, and 6),
         At least one 8-week induction regimen of tofacitinib (10 mg PO twice daily)
      Recurrence of symptoms during scheduled maintenance dosing following prior clinical benefit of the above biologics
      Note: Subjects who discontinued biologics or tofacitinib for reasons other than inadequate response as defined above or intolerance (e.g., change of insurance) must meet the criteria for intolerance or inadequate response to aminosalicylates, oral locally acting steroids, systemic steroids (prednisone or equivalent), and/or immunomodulators as defined above

5. If female, subject must meet either postmenopausal or permanently surgically sterile, or for women of childbearing potential, practicing birth control, prior to Baseline through at least 140 days after the last dose of study drug. Females of childbearing potential must have a negative serum pregnancy test result during Screening, and a negative urine pregnancy at Baseline. Females of non-childbearing potential (either postmenopausal or permanently surgically sterile) during Screening do not require pregnancy testing at Baseline.

6. Subject must be able and willing to give written informed consent and to comply with the requirements of this study protocol. In Japan, if the subject was <20 years old, a subject's parent or legal guardian must be willing to give written informed consent.

Main Exclusion:

1. Subject with a current diagnosis of Crohn's disease (CD), IBD-unclassified (IBD-U) or a history of radiation colitis or ischemic colitis.

Concomitant Medications and Treatments

2. Subject on oral UC-related antibiotics who has not been on stable doses for greater than, or discontinued within, 14 days prior to Baseline.

3. Subject on oral aminosalicylates who has not been on stable doses for greater than, or discontinued within, at least 14 days prior to Baseline.

4. Subject taking oral corticosteroids: Budesonide >9 mg/day; Beclomethasone >5 mg/day; Prednisone or equivalent >20 mg/day; or has not been on the current course for ≥14 days prior to Baseline and on a stable dose for ≥7 days prior to Baseline.

5. Subject on immunomodulators (AZA, 6-MP, MTX) who has not been on the course for ≥42 days prior to Baseline, and has not been on a stable dose for ≥35 days prior to Baseline.

Medications and Treatments During the Screening Period

6. Subject who received IV anti-infectives within 35 days prior to Baseline visit or oral anti-infectives (non-UC-related) within 14 days prior to the Baseline visit.

7. Subject who received any parenteral nutrition within 35 days prior to Baseline.

8. Subject who received any live bacterial or viral vaccination within 35 days (8 weeks for Japan) prior to Baseline.

9. Subject who received cyclosporine, tacrolimus, or mycophenolate mofetil within 35 days prior to Baseline.

10. Subject who received fecal microbial transplantation within 35 days prior to Baseline.

Prior Medications and Treatments

11. Subject who received any approved biologic agent (e.g., infliximab, adalimumab, golimumab, vedolizumab) within 8 weeks prior to Baseline, or tofacitinib within 35 days prior to the Baseline.

12. Subject with prior exposure to p40 inhibitors (e.g., ustekinumab [Stelara]) or p19 inhibitors (e.g., risankizumab).

13. Subject has been taking combination of two or more of the following oral budesonide, oral beclomethasone, and/or oral prednisone (or equivalent) simultaneously, with the exception of inhalers, within 14 days prior to Screening or during the Screening period.

14. Subject who received IV corticosteroids within 14 days prior to Screening or during the Screening period.

15. Subject who received therapeutic enema or suppository (i.e., rectal aminosalicylates/corticosteroids), other than required for endoscopy, within 14 days prior to endoscopy used for Screening or during the Screening period.

16. Subject who received apheresis (e.g., Adacolumn apheresis) s 60 days prior to Screening or during the Screening period.

17. Subject who has concomitant cannabis use either recreational or for medical reasons within 14 days prior to Baseline or any history of clinically significant drug, or alcohol abuse in the last 12 months.

UC Related

18. Extent of inflammatory disease limited to the rectum as assessed by screening endoscopy

19. Subject with currently known complications of UC such as: fulminant colitis, toxic megacolon, previous colectomy (total or subtotal), or any other manifestation that may require surgery while enrolled in the study.

20. Subject with ostomy or ileoanal pouch.

Safety

21. Subject who has a known hypersensitivity to risankizumab or the excipients of any of the study drugs or the ingredients of Chinese hamster ovary (CHO).

22. Subjects with the following chronic or active infections:

active, chronic, or recurrent infection that based on the Investigator's clinical assessment makes the subject unsuitable candidate for the study;

infection with C. difficile toxin as identified during Screening;

known infection with an intestinal pathogen;

infection with human immunodeficiency virus (HIV);

QuantiFERON®-TB test or Purified Protein Derivative (PPD) skin test, or both, according to local guidelines, were performed during Screening. QuantiFERON®-TB test was preferred for subjects who had received BCG vaccination or had been exposed to other Mycobacteria species. Subjects with a positive test result may participate in the study if further work up (according to local practice/guidelines) established conclusively that the subject had no evidence of active tuberculosis (subjects with active TB or history of active TB who had documented completion of a full course of anti-TB therapy may be allowed to enter the study after consultation with the AbbVie TA MD). If latent TB was established, TB prophylaxis/treatment was initiated and maintained according to local country guidelines;

Having active hepatitis B or hepatitis C defined as: HBV: hepatitis B surface antigen (HBs Ag) positive (+), or detected sensitivity on the HBV deoxyribonucleic acid (DNA) polymerase chain reaction (PCR) qualitative test for hepatitis B core antibody (HBc Ab) positive subjects; HCV: HCV ribonucleic acid (RNA) detectable in any subject with anti-HCV antibody (HCV Ab).

23. Subject with a previous history of dysplasia of the gastrointestinal tract or found to have dysplasia, other than completely removed low-grade dysplastic lesions, in any biopsy performed during the Screening endoscopy.

24. Subject with a known history of lymphoproliferative disease, including lymphoma, or signs and symptoms suggestive of possible lymphoproliferative disease, such as lymphadenopathy and/or splenomegaly.

25. Subject with or history of malignancy other than a successfully treated non-metastatic cutaneous squamous cell or basal cell carcinoma or localized carcinoma in situ of the cervix.

26. Subject who has severe, progressive, or uncontrolled renal, hepatic, hematological, endocrine, disorder or symptoms thereof.

27. Female subjects who is pregnant, breastfeeding, or is considering becoming pregnant during the study or for approximately 140 days after the last dose of study drug.

28. Subject who has any condition including any physical, psychological, or psychiatric condition, which in the opinion of the Investigator, would compromise the safety of the subject or the quality of the data and renders the subject an unsuitable candidate for the study.

29. Screening laboratory and other analyses show any of the following abnormal results:
aspartate transaminase (AST), alanine transaminase (ALT) >2×upper limit of the reference range;
white blood cell (WBC) count <3.0×10⁹/L;
total bilirubin ≥2 mg/dL; except for subjects with isolated elevation of indirect bilirubin relating to Gilbert syndrome;
estimated glomerular filtration rate by simplified 4-variable Modification of Diet in Renal Disease (MDRD) formula <30 ml/min/1.73 m²;
hemoglobin <8 g/dL;
platelets <100,000/μL; or
positive serum pregnancy test at the Screening visit or positive urine pregnancy test at the Baseline visit.

Investigational Product: Risankizumab

Doses: Risankizumab 1800 mg IV Q4W*, Risankizumab 1200 mg IV Q4W, Risankizumab 360 mg SC Q8W, or Risankizumab 180 mg SC Q8W.

Mode of Administration: Risankizumab solution for infusion (IV) or Risankizumab solution for injection (SC).

Reference Therapy: Placebo for Risankizumab.

Mode of Administration: Risankizumab solution for infusion (IV) or Risankizumab solution for injection (SC).

Duration of Treatment: 12 to 24 weeks

The study included a Screening period of up to 35 days and a double blind induction period of 12 weeks. All subjects who did not achieve clinical response at Week 12 were eligible to receive blinded risankizumab treatment over a subsequent 12 week period. There was a follow up call 140 days from the last dose of study drug to obtain information on any new or ongoing AEs for those subjects who did not enroll into Study M16-066 or discontinue from the study prematurely.

Criteria for Evaluation (Same for Sub-Study 4 and Sub-Study 5):

Clinical Remission per Adapted Mayo: stool frequency subscore (SFS) s 1 and not greater than baseline, rectal bleeding subscore (RBS)=0, and endoscopic subscore s 1;

Clinical Response per Adapted Mayo: decrease from Baseline ≥2 points and ≥30%, PLUS a decrease in RBS ≥1 or an absolute RBS ≤1; Clinical Response per Partial Adapted Mayo (without endoscopy): decrease from Baseline ≥1 points and ≥30%, PLUS a decrease in RBS ≥1 or an absolute RBS ≤1;

Clinical Remission per Full Mayo: Full Mayo score ≤2 with no subscore >1;

Endoscopic Improvement: endoscopy subscore of 0 or 1;

Endoscopic Remission: endoscopic subscore=0;

Histologic Remission: Geboes score of <2.0;

Mucosal Healing: endoscopic and histologic remission.

Note: Evidence of friability during endoscopy in subjects with otherwise “mild” endoscopic activity conferred an endoscopic subscore of 2.

Efficacy (Risankizumab Versus Placebo): The primary endpoint was the same for Sub-Studies 1 and 2.

Primary Endpoint: proportion of subjects with clinical remission per Adapted Mayo score at Week 12.

Sub-Study 4 Ranked Secondary Endpoints:

1. Proportion of subjects with endoscopic improvement at Week 12;

2. Proportion of subjects achieving clinical remission per Full Mayo score (defined as a Full Mayo score s 2 with no subscore >1) at Week 12 in subjects with a Full Mayo score of 6 to 12 at Baseline;

3. Proportion of subjects achieving clinical response per Adapted Mayo score at Week 12;

4. Proportion of subjects achieving clinical response per Partial Adapted Mayo score at Week 4;

5. Proportion of subjects with endoscopic remission at Week 12;

6. Proportion of subjects with hospitalizations through Week 12;

7. Proportion of subjects with mucosal healing at Week 12;

8. Change from Baseline to Week 12 in UC-Symptom Questionnaire (UC-SQ);

9. Change from Baseline to Week 12 in Inflammatory Bowel Disease Questionnaire (IBDQ);

10. Change from Baseline to Week 12 in Short Form-36;

11. Change from Baseline to Week 12 in Functional Assessment of Chronic Illness Therapy-Fatigue (FACIT-Fatigue);

12. Proportion of subjects with UC-related surgeries through Week 12.

Criteria for Evaluation (Same for Sub-Study 4 and Sub-Study 5)

Efficacy (Risankizumab Versus Placebo): The primary endpoint was the same for Sub-Studies 1 and 2.

Primary Endpoint:

Proportion of subjects with clinical remission per Adapted Mayo score at Week 12.

Sub-Study 4 Ranked Secondary Endpoints:

1. Proportion of subjects with endoscopic improvement at Week 12

2. Proportion of subjects achieving clinical remission per Full Mayo score (defined as a Full Mayo score [symbol] 2 with no subscore >1) at Week 12 in subjects with a Full Mayo score of 6 to 12 at Baseline

3. Proportion of subjects achieving clinical response per Adapted Mayo score at Week 12

4. Proportion of subjects achieving clinical response per Partial Adapted Mayo score at Week 4

5. Proportion of subjects with endoscopic remission at Week 12

6. Proportion of subjects with hospitalizations through Week 12

7. Proportion of subjects with mucosal healing at Week 12

8. Change from Baseline to Week 12 in UC-Symptom Questionnaire (UC-SQ)

9. Change from Baseline to Week 12 in Inflammatory Bowel Disease Questionnaire (IBDQ)

10. Change from Baseline to Week 12 in Short Form-36

11. Change from Baseline to Week 12 in Functional Assessment of Chronic Illness Therapy-Fatigue (FACIT-Fatigue)

12. Proportion of subjects with UC-related surgeries through Week 12

Sub-Study 5 Ranked Secondary Endpoints:

1. Proportion of subjects with endoscopic improvement at Week 12

2. Proportion of subjects achieving clinical remission per Full Mayo score (defined as a Full Mayo score [symbol] 2 with no subscore >1) at Week 12 in subjects with a Full Mayo score of 6 to 12 at Baseline

3. Proportion of subjects achieving clinical response per Adapted Mayo score at Week 12

4. Proportion of subjects achieving clinical response per Partial Adapted Mayo score at Week 4

5. Proportion of subjects who reported no abdominal pain at Week 12

6. Proportion of subjects who reported no bowel urgency at Week 12

7. Proportion of subjects with endoscopic remission at Week 12

8. Proportion of subjects with histologic endoscopic improvement of the mucosa at Week 12

9. Change from Baseline to Week 12 in Inflammatory Bowel Disease Questionnaire (IBDQ) total score

10. Change from Baseline to Week 12 in Functional Assessment of Chronic Illness Therapy-Fatigue (FACIT-Fatigue)

11. Proportion of subjects who reported no nocturnal bowel movements at Week 12

12. Proportion of subjects who reported no tenesmus at Week 12

13. Change from Baseline to Week 12 in number of fecal incontinence episodes per week

14. Change from Baseline to Week 12 in number of days per week with sleep interrupted due to UC symptoms

15. Proportion of subjects achieving clinical response per Adapted Mayo score at Week 12 in subjects with pancolitis at Baseline

16. Proportion of subjects with UC-related hospitalizations through Week 12

Pharmacokinetics (PK):

Serum risankizumab concentrations were determined from samples collected just prior to dosing at Weeks 4, 8, and 12/PD, and at Week 24 for subjects who underwent blinded risankizumab treatment during Induction Period 2.

Additionally, intensive pharmacokinetic assessment was performed in 24 subjects in Sub-Study 4 after the 3^{rd} induction dose (Weeks 8 to 12). For subjects who consented to the intensive pharmacokinetic assessment, in addition to the time points above, blood samples were collected at Week 8, immediately after completion of infusion and 2 hours post completion of infusion, and at Weeks 9, 10 and 11.

Immunogenicity:

Serum ADAs were determined from samples collected just prior to dosing at Baseline and Weeks 4, 8, and 12/PD, and at Week 24 for subjects who underwent blinded risankizumab treatment during Induction Period 2.

Safety:

Incidence of adverse events (AEs), changes in vital signs, physical examination results, and clinical laboratory data were assessed throughout the study.

Statistical Methods:

Sample Size Determination:

Sub-Study 4:

For Sub-Study 4 (Phase 2b portion of the study), a total of 240 subjects were equally randomized with 1:1:1:1 ratio to three risankizumab treatment groups (600 mg, 1200 mg and 1800 mg IV Q4W) and the placebo group. Assuming clinical remission rate of 7% in the placebo arm and maximum of 25% in at least one of the risankizumab treatment groups at Week 12, a sample size of 60 subjects per treatment group was sufficient to test for the presence of a dose response signal with an average power of approximately 87% at 5% level of significance (one-sided), via modeling using Multiple comparison procedure and modeling (MCP-Mod) approach (Pinheiro J, Bornkamp B, and Bretz F. (2006) J. Biopharm. Stat. 16:639-656; Bretz F, Pinheiro J C, and Branson M. (2005) Biometrics 61:738-748).

Sub-Study 5:

For Sub-Study 5, a total of 966 subjects were allocated to risankizumab 1200 mg IV dose or placebo in a randomization ratio of 2:1. The sample size has been reassessed after analyzing the combined PK, safety and efficacy results from Sub-Study 4. It was determined to provide adequate powers for the primary endpoint and selected ranked secondary endpoints and adequate responders to meet the sample size requirement for Study M16-066. Assuming clinical remission rate of 6% in the placebo arm and 16% of the risankizumab treatment arms at Week 12, a sample size of 644:322 subjects per arm provides at least 90% power to detect the 10% treatment difference in the primary endpoint using two-sided Miettinen and Nurminen test at a 0.05 significant level.

Analysis Sets:

For both Sub-Studies 1 and 2, efficacy analysis was based on Intent-to-Treat (ITT) analysis set and Safety analysis was based on safety analysis set. Subjects who received treatment in Induction Period 2 after Week 12 in both Sub-Study 4 and Sub-Study 5 were analyzed separately for exploratory purpose.

Sub-Study 4:

The pairwise comparison between each risankizumab treatment group and placebo was performed using the 2-sided Cochran-Mantel-Haenszel (CMH) test and stratified by baseline corticosteroid use (yes vs no) and baseline Adapted Mayo score (≤7 vs >7).

Non-responder imputation method (NRI) was used for all missing categorical endpoints as the primary imputation method.

Continuous secondary efficacy variables were analyzed using a Mixed-Effect Model Repeated Measures (MMRM) method and Analysis of Covariance (ANCOVA) model using LOCF imputation method.

Categorical secondary efficacy variables were analyzed using the MN test with CMH weight controlling for stratification variables. NRI and observed case analyses were used.

Sub-Study 5:

All efficacy comparisons between risankizumab and placebo were based on corresponding ITT analysis set.

The comparisons between each risankizumab treatment group and placebo for the primary efficacy endpoint clinical remission rate at Week 12 were performed using 2-sided the Miettinen and Nurminen (MN) test with Cochran-Mantel-Haenszel (CMH) weight and were stratified by number of prior failed biologics, (0, 1 vs >1), baseline corticosteroid use (yes vs no), and baseline Adapted Mayo score ( [symbol] 7 vs >7). Non-responder imputation while incorporating multiple imputation to handle missing data due to COVID-19 (NRI-C) was used for all missing categorical endpoints as the primary imputation method.

Continuous secondary efficacy variables were analyzed using a Mixed-Effect Model Repeated Measures (MMRM) method.

Categorical secondary efficacy variables were analyzed using the MN test with CMH weight controlling for stratification variables. NRI-C and observed case analyses were used.

Pharmacokinetics and Immunogenicity:

Serum risankizumab concentrations were summarized at each time point for each dosing regimen using descriptive statistics.

ADA incidence was summarized by cohort and study visits. ADA titers were tabulated for each subject at the respective study visits. The effect of ADA on risankizumab pharmacokinetics, efficacy and/or safety variable(s) and/or any additional analyses were explored.

Safety:

Treatment Emergent Adverse events (TEAEs), laboratory data and vital signs were the primary safety parameters in this study. All safety comparisons were performed between treatment groups based on the corresponding safety analysis set.

An overview of TEAEs, including TEAEs of special interest such as serious infection, malignancies, major adverse cardiovascular events, systemic hypersensitivity reactions/infusion reactions, TEAEs leading to death and TEAEs leading to premature discontinuation, TEAEs by Medical Dictionary for Drug Regulatory Activities (MedDRA version 18.1 or later) preferred term and system organ class, TEAEs by maximum relationship to study drug, and TEAEs by maximum severity was summarized by number and percentage. Treatment group differences in the overall incidence of TEAEs were assessed with Fisher's exact test for each preferred term.

Changes in laboratory data were described using statistical characteristics and compared between-treatment groups which was performed using a one-way Analysis of Variance (ANOVA). In addition, shift tables and listings were provided for abnormal values, whereby the normal range of the analyzing laboratory was used. Vital signs were analyzed similarly.

### Example 10: UC Maintenance Study M16-066

A multicenter, randomized, double-blind, placebo-controlled 52-week maintenance study of the efficacy and safety of risankizumab in subjects with ulcerative colitis who responded to induction treatment in M16-067 was carried out.

Objective:

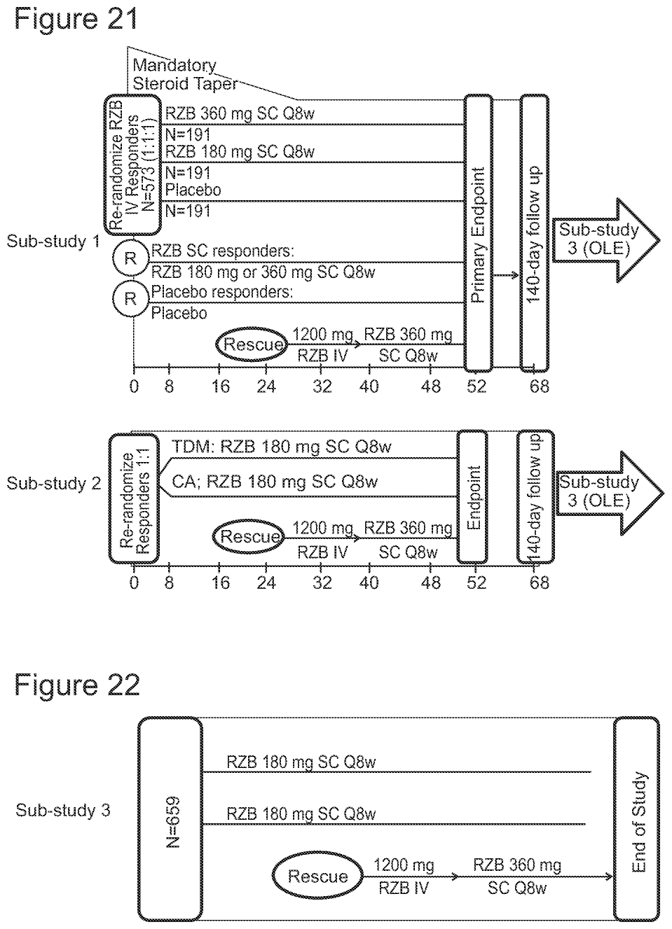

Sub-Study 4 (FIG. 21): Randomized, double-blind, placebo-controlled maintenance

To evaluate the efficacy and safety of risankizumab versus placebo as maintenance therapy in subjects with moderately to severely active ulcerative colitis (UC) who responded to IV risankizumab induction treatment in Study M16-067.

Sub-Study 5 (FIG. 21): Randomized, Exploratory Maintenance

To evaluate the efficacy and safety of two different dosing regimens for risankizumab (therapeutic drug monitoring vs clinical assessment for dose escalation) as maintenance therapy in subjects with moderately to severely active UC who responded to induction treatment in Study M16-067.

Sub-Study 6 (FIG. 22): Open-Label Long Term Extension

To evaluate long-term safety of risankizumab in subjects who completed Sub-Study 4 or 2. Additional objectives were to further investigate long-term efficacy and tolerability of risankizumab.

Study Sites: Approximately 400 sites worldwide.

Study Population:

The study enrolled subjects who have completed Study M16-067 and have achieved clinical response, defined as decrease from Baseline of induction study of Adapted Mayo score [symbol] 2 points and [symbol] 30%, PLUS a decrease in rectal bleeding sub-score (RBS) [symbol] 1 or an absolute RBS [symbol] 1.

Main Entry Criteria for Study M16-067:

Males and females [symbol] 18 to [symbol] 80 years of age, or minimum age of adult consent according to local regulations, at the Baseline Visit. Where locally permissible [symbol] 16 to <18 years of age who met the definition of Tanner stage 5 development.

Moderately to severely active UC, defined as an adapted Mayo Score of 5 to 9 points and endoscopy subscore of 2 to 3, as confirmed by a centrally read endoscopy.

Number of Subjects to be Enrolled:

Approximately 942 in total for both Sub-Study 4 and Sub-Study 5.

Sub-Study 4 enrolled first. Subjects who achieved clinical response to study drug after induction Study M16-067 continued to enroll until approximately 573 subjects who achieved clinical response, confirmed by central review of Mayo endoscopic score, to IV risankizumab were randomized.

Sub-Study 5 enrolled the remaining subjects who achieved clinical response to study drug after induction Study M16-067.

Sub-Study 4 enrolled approximately 458 bio-IR subjects. Once approximately 458 bio-IR subjects have been enrolled, the remaining bio-IR subjects with clinical response at the end of induction entered Sub-Study 5.

Subjects who complete Sub-studies 1 or 2 were eligible for Sub-Study 6.

Methodology:

This was a Phase 3, multicenter study that consists of three sub-studies: Sub-Study 4 was a 52 week randomized, double-blind, placebo-controlled maintenance study. Sub-Study 5 was a 52 week randomized, exploratory maintenance study. Sub-Study 6 was an open-label (OL) long-term extension for subjects who completed Sub-Study 4 or 2.

Hereafter, Baseline was defined as the Baseline Visit of the induction Study M16-067, and Week 0 was defined as the first study visit in Sub-Study 4 or Sub-Study 5 of Study M16-066. The final visit of Study M16-067 (Week 12 or Week 24) was considered as the Week 0 visit of Study M16-066 Sub-studies 1 or 2. The duration of Sub-studies 1 or 2 may be up to 68 weeks, including a 52-week maintenance period and a 140-day follow-up period (except for subjects who continue in Sub-Study 6) from last dose of study drug administered at Week 48. Sub-Study 6 lasted until 300 weeks of individual follow-up or until the study was discontinued, whichever was earlier.

Sub-Study 4 (Randomized, Double-Blind, Placebo-Controlled Maintenance)

The first approximately 573 subjects who achieved clinical response to IV risankizumab at the end of Study M16-067 were re-randomized in a 1:1:1 ratio to one of the following three treatment groups:

Group 1: Risankizumab 180 mg subcutaneous (SC) Q8W (n=150)

Group 2: Risankizumab 360 mg SC Q8W (n=150)

Group 3: Placebo (n=150)

Subjects continued to enroll until a minimum of 573 subjects with clinical response to IV risankizumab confirmed by central reader have been enrolled.

Only subjects achieving clinical response after IV risankizumab at Week 12 of the induction study or Week 24 of Induction Period 2 in the induction study were randomized to Group 1, 2, or 3 in the Sub study 1 of the Study M16-066. Subjects achieving clinical response after SC risankizumab at Week 24 of Induction Period 2 in the induction study were eligible to enroll, but were assigned by Interactive Response Technology (IRT) to receive blinded risankizumab 180 mg or 360 mg SC at the same dose they received during Induction Period 2 of the induction study Q8W and were excluded from the primary analysis.

Subjects with clinical response to placebo in Study M16-067 were assigned by IRT to continue to receive blinded placebo and were excluded from the primary efficacy analysis.

Subject randomization was stratified based on last IV risankizumab induction dose and clinical remission per Adapted Mayo score (per local read) from the last visit of Study M16-067.

Sub-Study 4 enrolled approximately 458 bio-IR subjects. Once approximately 458 bio-IR subjects have been enrolled, the remaining bio-IR subjects with clinical response at the end of induction entered Sub-Study 5.

Subjects who demonstrated inadequate response (IR) during Sub-Study 4 may have received OL risankizumab rescue therapy starting at the Week 16 Visit based upon increased symptom activity and/or endoscopic confirmation of inflammation. Details are described in the “Risankizumab Rescue Therapy” section.

Sub-Study 5 (Randomized, Exploratory Maintenance):

Enrollment for Sub-Study 5 initiated after completion of enrollment for Sub-Study 4. Subjects who achieved clinical response at the end of Study M16-067, regardless of induction or Induction Period 2 treatment, were randomized 1:1 into an exploratory 52 week maintenance study for comparison between two treatment regimens: clinical assessment (CA) for dose escalation and therapeutic drug monitoring (TDM) for dose escalation (treating to target levels of risankizumab).

In order to ensure subjects have approached steady state for risankizumab by Week 16 and to maintain the double blind from induction, treatment at the Week 0 Visit was blinded:
Subjects with clinical response to risankizumab received risankizumab 180 mg SC and placebo intravenous (IV).
Subjects with clinical response to placebo received placebo SC and risankizumab Selected Dose IV.

All subjects received OL risankizumab 180 mg SC starting at Week 8. Risankizumab rescue therapy was administered at any time on or after the Week 16 visit and was allowed as per IR criteria described in the “Risankizumab Rescue Therapy” section. Subjects were evaluated at each scheduled visit for risankizumab rescue therapy.

Subjects in the CA and TDM arms received risankizumab rescue therapy based on the criteria in Table 5.

Results of serum risankizumab analysis were provided to the site in approximately 2 weeks. All risankizumab samples were shipped the same day they were collected at Weeks 16, 24, 32, 40, and 48 to ensure adequate processing time to determine serum concentration levels needed for dosing adjustments.

Sub-Study 4 and 2

Subjects used the same subject diary that was dispensed for Studies M16-067. Subjects were also dispensed a patient information card for Study M16-066. Visits occurred at Weeks 0, 8, 16, 24, 32, 40, 48, and 52/Premature Discontinuation (PD) to collect clinical and laboratory assessments of disease activity. Additionally, subjects completed symptom, quality of life (QoL) and work productivity questionnaires throughout the study. Clinical labs including, but not limited to, urinalysis, chemistry and hematology, high-sensitivity C-reactive protein (hs-CRP), serum risankizumab concentrations, and serum anti-drug antibody (ADA) levels were collected at the visits indicated. In addition, stool samples for calprotectin (FCP) analysis were collected before starting bowel preparations for endoscopy. An endoscopy occurred at the Week 52/PD visit and was evaluated using Mayo endoscopic score, confirmed by a central reader. Biopsy to rule out dysplasia/malignancy may have been performed at the same time as the endoscopy. Optional exploratory research samples may have been taken during Sub-Study 4. Subjects who terminated the study early or did not continue into Sub-Study 6 were discontinued and had an additional 140 days of safety follow-up from the last dose administration of study drug.

Sub-Study 6: OL Extension

Subjects who completed Sub-Study 4 or 2 were eligible to enter Sub-Study 6, an OL extension study. All study activities, including endoscopy at Week 52, were completed for a subject to enter Sub-Study 6. Subjects received OL risankizumab based on their assignment during Sub-Study 4 or 2:
Subjects completing Sub-Study 4 or 2 without receiving risankizumab rescue therapy: risankizumab 180 mg SC Q8W.
Subjects completing Sub-Study 4 or 2 and receiving risankizumab rescue therapy: risankizumab 360 mg SC Q8W.

Subjects who continued in Sub-Study 6 had OL risankizumab syringes dispensed at Week 56 at an onsite visit. At the Week 56 Visit, subjects enrolling from Sub-Study 4 who have not received rescue therapy in Sub-Study 4 and subjects enrolling from the induction study M16-067 directly into Sub-Study 6 were monitored for 1 hour after SC administration of drug. This ensured that subjects who received placebo during induction and Sub-Study 4 and receive risankizumab SC for the first time were monitored for any injection related events. Additional Visits occurred every 24 weeks (where locally permitted) starting at Week 56 and enough risankizumab syringes were dispensed for at home injections (if allowed per local requirements) between visits. Endoscopies occurred every 96 weeks starting at Week 152. The same subject diary was used as during Substudy 1 or 2 or during the induction Study M16-067.

Subjects who demonstrated IR during Sub-Study 6 were eligible to receive OL risankizumab rescue therapy as described in the “Risankizumab Rescue Therapy” section. Doses of UC-related antibiotics, aminosalicylates, corticosteroids or immunomodulators may have been changed at the discretion of the Investigator and documented in the appropriate eCRF.

Subjects may have started UC-related antibiotics, aminosalicylates, corticosteroids, or immunomodulators (AZA, 6-MP, or MTX) during Sub-Study 6 and have the doses changed at the discretion of the Investigator.

Risankizumab Rescue Therapy:

Definition of Inadequate Response (IR)

Subjects in Sub-Study 4, the CA arm of Sub-Study 5, and Sub-Study 6 received risankizumab rescue therapy based upon:
1. clinical symptoms: RBS at least 1 point greater than the Week 0 value, or the value at the final visit in induction study M16-067 for subjects enrolling directly into Sub-Study 6, on two consecutive visits 7-14 days apart, or
2. endoscopic activity: Mayo endoscopic sub-score, as scored by the site Investigator, of 2 or 3.

Subjects in the TDM arm of Sub-Study 5 received risankizumab rescue therapy based upon the criteria listed above for clinical symptoms and endoscopic confirmation of inflammation, as well as serum risankizumab levels as indicated in Table 5. If the investigator had a reasonable suspicion of a gastrointestinal infection they should ensure these subjects were excluded prior to dose escalation.

Subjects in Sub-Study 5 were evaluated at each Study Visit for IR. For subjects in the TDM arm, the serum risankizumab level from the prior study visit was used for evaluation, except at the Week 16 Study Visit where the dose escalation algorithm used the serum risankizumab level from the same visit*. *Note: Once Week 16 serum risankizumab levels were available, sites were notified if the subject needs to return to clinic to receive rescue therapy.

TABLE 5
IR Criteria to Receive Risankizumab Rescue Therapy

| | | |
|---|---|---|
| | | Risankizumab |
| [RZB] Serum | Symptom or Endoscopic | Rescue |
| (μg/mL) | Activity^{1, 2} | Therapy? |

Sub-Study 4, CA Arm Sub-Study 5, Sub-Study 6

| | | |
|---|---|---|
| N/a | Low | No |
| N/a | High | Yes |

TDM Arm Sub-Study 5

| | | |
|---|---|---|
| <4 | Any | Yes |
| ≥4 to <8 | Low | No |
| ≥4 to <8 | High | Yes |
| ≥8 | Any | No³ |

¹Clinical symptoms: RBS at least 1 point greater than the Week 0 value, or the value at the final visit in induction study M16-067 for subjects enrolling directly into Sub-Study 6, on two consecutive visits 7-14 days apart, or;
²Endoscopic activity: Mayo endoscopic sub-score, as scored by the site Investigator, of 2 or 3.
³Subjects who have high symptom activity, high endoscopic activity, and high serum levels of risankizumab should be withdrawn from the study if they have been on a stable dose of risankizumab for >8 weeks.

Risankizumab Rescue Therapy Dose and Timing of Visits

If a subject has IR as defined above, they may have received risankizumab rescue therapy, which was one dose of 1200 mg IV followed by 360 mg SC through the end of the trial. Rescue Visit was defined as the date on which the subject was administered risankizumab IV. All attempts were made to keep subjects on their original SC dosing regimen of Q8W intervals. This aligned with scheduled Study Visits in Sub-Study 4 and 2 and every third Study Visit in Sub-Study 6 (Q24w visits).

The study medication schedule when the subjects move to risankizumab rescue depended on when the Rescue Visit occurred:

If the Rescue Visit occurred within the 7 days prior to a scheduled study visit or on the same day of the scheduled visit, both visits (rescue and scheduled) were combined and the subject only received risankizumab IV at the site.

If the Rescue Visit occurred after the planned scheduled visit, within the 7 day window, and the subject had not received the SC dose yet, both visits (rescue and scheduled) were also combined and the subject only received risankizumab IV at the site.

If the Rescue Visit occurred after the scheduled study visit and the subject had already received the SC dose at the scheduled visit, the subject received risankizumab IV at the Rescue Visit.

After the IV dose, the subject received 360 mg risankizumab SC at the next scheduled dose (may be less than 8 weeks) and Q8W thereafter through the end of the study.

During Sub-Study 4 and 2, the evaluation for risankizumab rescue therapy may have begun at the Week 16 Visit. Subjects may had up to two Rescue Visits during Sub-Study 4 or 2, but must have met the definition of IR as described above for each Rescue Visit and the Rescue Visits must be >16 weeks apart.

During Sub-Study 6, subjects may have received up to 2 Rescue Visits per year and the Rescue Visits were >16 weeks apart. Subjects may have received up to 3 Rescue Visits in total during Sub-Study 6.

Diagnosis and Main Criteria for Inclusion/Exclusion:

Main Inclusion:

1. Entry and completion of Study M16-067. Completion includes the final endoscopy of Study M16-067.

2. Achieved clinical response, defined as decrease in Adapted Mayo Score [symbol] 2 points and [symbol] 30% from Baseline, PLUS a decrease in RBS [symbol] 1 or an absolute RBS [symbol] 1 at the last visit of Study M16-067.

3. If female, subject must be either postmenopausal, permanently surgically sterile, or for Women of Childbearing Potential, continue practicing methods of birth control through at least 140 days after the last dose of study drug.

4. Subject must be able and willing to give written informed consent and to comply with the requirements of this study protocol, including self-administration or care-giver administration of SC injections. In Japan, if the subject was <20 years old, a subject's parent or legal guardian must be willing to give written informed consent.

Main Exclusion:

1. Subject was considered by the Investigator, for any reason, to be an unsuitable candidate for the study. Subjects should not be enrolled in Study M16-066 if high grade colonic dysplasia or colon cancer was discovered at the endoscopy performed at the final visit of Study M16-067.

2. Subject who has a known hypersensitivity to risankizumab or the excipients of any of the study drugs or the ingredients of CHO, or had an AE during Study M16-067 that in the Investigator's judgment makes the subject unsuitable for this study.

3. Confirmed positive urine pregnancy test at the Final Visit of Study M16-067.

4. Subject was not in compliance with prior and concomitant medication requirements throughout Study M16-067.

5. Subject with any active or chronic recurring infections based on the Investigator's assessment makes the subject an unsuitable candidate for the study.

6. Have a known history of lymphoproliferative disease, including lymphoma, or signs and symptoms suggestive of possible lymphoproliferative disease, such as lymphadenopathy and/or splenomegaly.

Investigational Product: Risankizumab

Doses: Risankizumab 360 mg SC Q8W, Risankizumab 180 mg SC Q8W, or Risankizumab 1200 mg IV

Mode of Administration: Risankizumab solution for infusion (IV) or Risankizumab solution for injection (SC).

Reference Therapy: Placebo for risankizumab.

Dose: Placebo: N/A.

Mode of Administration: Risankizumab solution for infusion (IV) or Risankizumab solution for injection (SC).

Duration of Treatment: Sub-Study 4 and 2 had a duration of 52 weeks and Sub-Study 6 continued until discontinuation or approval and reimbursement (if applicable) for risankizumab were available in site's jurisdiction. Subjects who prematurely discontinued the study had a 140-day follow-up phone call for safety purposes from the last administration of study drug.

Endpoint Definitions*:

Clinical Remission per Adapted Mayo: stool frequency subscore (SFS) [symbol] 1 and not greater than baseline, rectal bleeding subscore (RBS)=0, and endoscopic subscore [symbol] 1;

Clinical Response per Adapted Mayo: decrease from Baseline [symbol] 2 points and [symbol] 30% from Baseline, PLUS a decrease in RBS [symbol] 1 or an absolute RBS [symbol] 1;

Clinical Remission per Partial Adapted Mayo: stool frequency subscore (SFS) [symbol] 1 and not greater than baseline, rectal bleeding subscore (RBS)=0;

Clinical Response per Partial Adapted Mayo: decrease from Baseline [symbol] 1; points and [symbol] 30% from Baseline, PLUS a decrease in RBS [symbol] 1 or an absolute RBS [symbol] 1;

Clinical Remission per Full Mayo: Full Mayo score [symbol] 2 with no subscore >1;

Endoscopic Improvement: endoscopic subscore [symbol] 1;

Endoscopic Remission: endoscopic subscore=0;

Histologic Remission: Geboes score of <2.0;

Mucosal Healing: endoscopic and histologic remission.

(* Notes: Baseline refers to the Baseline of induction Study M16-067. Evidence of friability during endoscopy in subjects with otherwise “mild” endoscopic activity confers an endoscopic subscore of 2.)

Criteria for Evaluation:

Efficacy:

Sub-Study 4 (Risankizumab vs Placebo)

Primary Endpoint: Proportion of subjects with clinical remission per Adapted Mayo score at Week 52.

Ranked Secondary Endpoints:

1. Proportion of subjects with endoscopic improvement at Week 52.

2. Proportion of subjects achieving clinical remission per Full Mayo score at Week 52 in subjects with a Full Mayo score of 6 to 12 at Baseline (of induction).

3. Proportion of subjects who discontinued corticosteroid use at Week 52 in subjects taking steroids at Baseline (of induction).

4. Proportion of subjects with clinical remission per Adapted Mayo score at Week 52 in subjects with clinical remission at Week 0.

5. Proportion of subjects who discontinued corticosteroid use, remained corticosteroid free for 90 days and achieved clinical remission at Week 52 in subjects taking steroids at Baseline (of induction).

6. Proportion of subjects with endoscopic improvement at Week 52 in subjects with endoscopic improvement at Week 0.

7. Proportion of subjects with clinical response per Adapted Mayo score at Week 52.

8. Proportion of subjects achieving histologic-endoscopic mucosal improvement at Week 52.

9. Proportion of subjects with endoscopic remission at Week 52.

10. Proportion of subjects with UC-related hospitalizations through Week 52.

11. Proportion of subjects with histologic remission at Week 52.

12. Proportion of subjects who reported no abdominal pain at Week 52.

13. Proportion of subjects who reported no bowel urgency at Week 52.

14. Proportion of subjects with mucosal healing at Week 52.

15. Change from Baseline (of induction) to week 52 in Inflammatory Bowel Disease Questionnaire (IBDQ) total score.

16. Proportion of subjects with UC-related surgeries through Week 52.

17. Change from Baseline (of induction) to week 52 in FACIT-Fatigue.

18. Proportion of subjects with clinical response per Adapted Mayo score at Week 52 in subjects with pancolitis at Baseline.

19. Proportion of subjects who reported no nocturnal bowel movements at Week 52.

20. Proportion of subjects who reported no tenesmus at Week 52.

21. Change from Baseline (of induction) to Week 52 in number of fecal incontinence episodes per week.

22. Change from Baseline (of induction) to week 52 in number of days over a week with sleep interrupted due to UC symptoms.

Pharmacokinetics:

Serum risankizumab concentrations were determined from samples collected just prior to dosing at Week 0, Week 16, Week 32, Week 48, Week 52 during Sub-studies 1 and 2 and every 24 weeks/PD during Sub-Study 6. Samples were also collected at the time of initiation of risankizumab rescue therapy and unscheduled visits. Additionally, for subjects in Sub-Study 5 samples were also collected at Week 8, Week 24 and Week 40.

Immunogenicity:

Serum ADA was determined from samples collected just prior to dosing at Week 0, Week 16, Week 32, Week 48, Week 52 during Sub-studies 1 and 2 and every 24 weeks/PD during Sub-Study 6. Samples were also collected at the time of initiation of risankizumab rescue therapy and unscheduled visits.

Safety:

Incidence of adverse events (AEs), changes in vital signs, physical examination results, and clinical laboratory data were assessed throughout the study.

Statistical Methods:

Efficacy:

For Sub-Study 4, the sample size was based on the expected proportion of subjects who achieve clinical remission per Adapted Mayo score at Week 52. Assuming clinical remission rate of 22% in the placebo arm and 42% in one of the risankizumab treatment arms at Week 52, a sample size of 191 subjects in placebo and 191 subjects in each of the risankizumab groups had more than 90% power to detect the 20% treatment difference in the primary endpoint between a risankizumab dose and placebo using two sided test at a 0.025 significant level with multiplicity adjustment.

No power calculation was conducted for the Sub-Study 5 and Sub-Study 6 samples size. Once Sub-Study 4 has completed enrollment, enrollment for Sub-Study 5 opens. Subjects who complete Sub-Study 4 or 2 may have entered Sub-Study 6, an OL extension study.

Efficacy analysis was based on Intent-to-Treat (ITT) analysis set and safety analysis was based on safety analysis set.

The primary efficacy analysis was performed to compare each risankizumab dose group and placebo group in Sub-Study 4 at Week 52. The analyses for other cohorts in Sub-Study 4, Sub-Study 5 and Sub study 3 were also conducted for exploratory purpose.

The comparison between risankizumab treatment groups and placebo for the primary efficacy endpoint was performed using the 2-sided Miettinen and Nurminen (MN) test with Cochran-Mantel-Haenszel (CMH) weight stratified by induction baseline bio-IR status (bio-IR vs non-bio-IR), last IV risankizumab induction dose and clinical remission per Adapted Mayo score (per central read) from the last visit of Study M16-067. Non-responder imputation method (NRI) was used for all missing primary efficacy endpoints as the primary imputation method.

In general, continuous secondary efficacy variables were analyzed using a Mixed-Effect Model Repeated Measures (MMRM).

Categorical secondary efficacy variables were analyzed using the CMH test controlling for stratification variables. NRI for missing data and observed case were used.

Pharmacokinetics and Immunogenicity:

Serum risankizumab concentrations were summarized at each time point for each dosing regimen using descriptive statistics.

ADA incidence was summarized by cohort and study visits. ADA titers were tabulated for each subject at the respective study visits.

Safety:

Treatment Emergent Adverse events (TEAEs), laboratory data and vital signs were the primary safety parameters in this study. All safety comparisons were performed between treatment groups using the safety analysis set.

An overview of TEAEs, including TEAEs of special interest such as serious infection, malignancies, major adverse cardiovascular events, systemic hypersensitivity reactions/infusion reactions, TEAEs leading to death and TEAEs leading to premature discontinuation, TEAEs by Medical Dictionary for Drug Regulatory Activities (MedDRA version 18.1 or later) preferred term and system organ class, TEAEs by maximum relationship to study drug, and TEAEs by maximum severity were summarized by number and percentage. Treatment group differences in the overall incidence of TEAEs were assessed with Fisher's exact test for each preferred term.

Changes in laboratory data were described using statistical characteristics and compared between treatment groups which was performed using a one-way Analysis of Variance (ANOVA). In addition, shift tables and listings were provided for abnormal values, whereby the normal range of the analyzing laboratory was used. Vital signs were analyzed similarly.

### Example 11: UC Induction Study M16-067 (Phase 2b) Results

1.0 Executive Summary:

M16-067 was an operationally seamless Phase 2b13 study designed to assess the safety and efficacy of risankizumab in subjects with moderately to severely active ulcerative colitis (UC) who have failed prior biologic therapy. The Phase 2b study of M16-067 was designed to identify the optimal induction dose for further study in the Phase 3 portion of the protocol. Doses of 600 mg, 1200 mg, and 1800 mg IV risankizumab at Weeks 0, 4, and 8 were evaluated. The primary endpoint was analyzed at Week 12. All three doses demonstrated higher clinical remission rates compared to placebo. The overall safety profile was consistent with the known safety profile of risankizumab, with no dose dependency.

Efficacy:

The rates of clinical remission per adapted mayo score at week 12 (primary endpoint), were 1.7%, 8.2%, 11.5% and 8.6% in placebo, risankizumab 600 mg, 1200 mg and 1800 mg groups, respectively. No prespecified dose response models were statistically significant based on the Multiple Comparison Procedure-Modeling (MCP-Mod) method. All risankizumab doses achieved higher remission rates compared to placebo; both the 1200 mg and 1800 mg groups achieved nominal p-values <0.1 (two-sided) based on pairwise comparisons.

All three risankizumab dose groups achieved higher response rates compared to placebo for clinical remission per full mayo score, clinical response per adapted mayo score, endoscopic remission and change from baseline in IBDQ at week 12.

Safety:

The overall incidences of AEs, serious AEs, severe AEs and AEs leading to discontinuation were generally balanced across different treatment groups with no exposure-dependent worsening in AEs, SAEs, infections, or serious infections.

No new safety risks were identified, and the overall safety profile was consistent with the known safety profile of risankizumab.

Pharmacokinetics and Immunogenicity:

Risankizumab showed a dose-dependent increase in exposure across the three doses evaluated, indicating linear PK as expected from previous studies.

Immunogenicity to risankizumab was low (Approximately 1% subjects developed treatment-emergent anti-risankizumab antibodies or neutralizing antibodies) with no dose-dependency and no impact on exposure.

2.0 Demographic, Baseline Characteristics, and Subject Disposition

Key demographics and baseline characteristics were generally balanced between treatment arms.

In Sub-Study 4, a total of 240 subjects were randomized, and all received study drug. Discontinuation rates were generally low (<10%) in the risankizumab groups. The highest discontinuation rate was observed in placebo group (11.7%).

3.0 Efficacy

3.1 Pairwise Comparison

In pairwise comparisons, all risankizumab doses achieved higher remission rates compared to placebo; both the 1200 mg and 1800 mg groups achieved nominal p-values <0.1; see Table 6. The results of selected secondary endpoints are presented in Table 7.

TABLE 6
Analysis of Primary Efficacy Endpoint (ITT1A, NRI)

| | | Risankizumab | | |
|---|---|---|---|---|
| | Placebo IV | 600 mg IV | 1200 mg IV | 1800 mg IV |
| | (N = 60) | (N = 61) | (N = 61) | (N = 58) |
| Endpoint | n (%) | n (%) | n (%) | n (%) |
| Clinical Remission per Adapted | 1 (1.7) | 5 (8.2) | 7 (11.5) | 5 (8.6) |
| Mayo Score¹ (Week 12) | | | | |
| Risk Difference² | | 6.5 | 9.8 | 7.0 |
| Two-sided p-value | | 0.109 | 0.022* | 0.074 + |
| 90% Confidence Interval | | (−0.2, 12.7) | (2.9, 17.7) | (0.6, 13.5) |

ITT1A: Intent-to-treat population consists of all randomized subjects who received at least on dose of study drug in Sub-Study 4 period 1.
*p < 0.05;
+ p < 0.1;
NRI: Non-responder imputation
¹Clinical remission per Adapted Mayo score: stool frequency subscore (SFS) ≤1 and not worse than BL, rectal bleeding subscore (RBS) of 0, and endoscopic subscore ≤1.
²difference = risankizumab group − placebo group

TABLE 7
Analysis of Selected Secondary Efficacy Endpoints (ITT1A, NRI)

| | | | Risankizumab | | | | | |
|---|---|---|---|---|---|---|---|---|
| | Placebo IV | | 600 mg IV | | 1200 mg IV | | 1800 mg IV | |
| | (N = 60) | | (N = 61) | | (N = 61) | | (N = 58) | |
| Endpoints | n (%) | | n (%) | | n (%) | | n (%) | |
| Endoscopic Improvement¹ (Week 12) | 4 | (6.7) | 12 | (19.7) * | 9 | (14.8) | 9 | (15.5) |
| Clinical Remission per Full Mayo Score² | 0 | | 4 | (6.8) * | 5 | (8.5) * | 3 | (5.7) + |
| (Week 12) | | | | | | | | |
| Clinical Response per Adapted Mayo | 13 | (21.7) | 26 | (42.6) ** | 29 | (47.5) *** | 31 | (53.4) *** |
| Score³ (Week 12) | | | | | | | | |
| Clinical Response per Partial Adapted | 15 | (25.0) | 20 | (32.8) | 28 | (45.9) ** | 22 | (37.9) |
| Mayo Score⁴ (Week 4) | | | | | | | | |
| Endoscopic Remission⁵ (Week 12) | 0 | | 5 | (8.2) * | 3 | (4.9) + | 4 | (6.9) * |
| Change from Baseline in IBDQ (Week 12) | 19.41 | (4.429) | 35.43 * | (4.473) | 38.53 ** | (4.301) | 38.82 ** | (4.416) |

*** p < 0.001;
** p < 0.01;
* p < 0.05;
+ p < 0.1;
NRI: Non-responder imputation
¹Endoscopic improvement: endoscopy subscore of 0 or 1.
²Clinical remission per Full Mayo score: Full Mayo score ≤2 with no subscore >1; based on subjects with a Full Mayo score of 6 to 12 at Baseline
³Clinical response per Adapted Mayo score: Decrease from baseline in the Adapted Mayo score ≥2 points and ≥30% from baseline, PLUS a decrease in rectal bleeding subscore (RBS) ≥1 or an absolute RBS ≥1.
⁴Clinical response per Partial Adapted Mayo score (RBS and SFS): Decrease from baseline in the Partial Adapted Mayo score ≥1 point and ≥30% from baseline, PLUS a decrease in RBS ≥1 or an absolute RBS ≤1.
⁵Endoscopic remission: endoscopic subscore of 0.

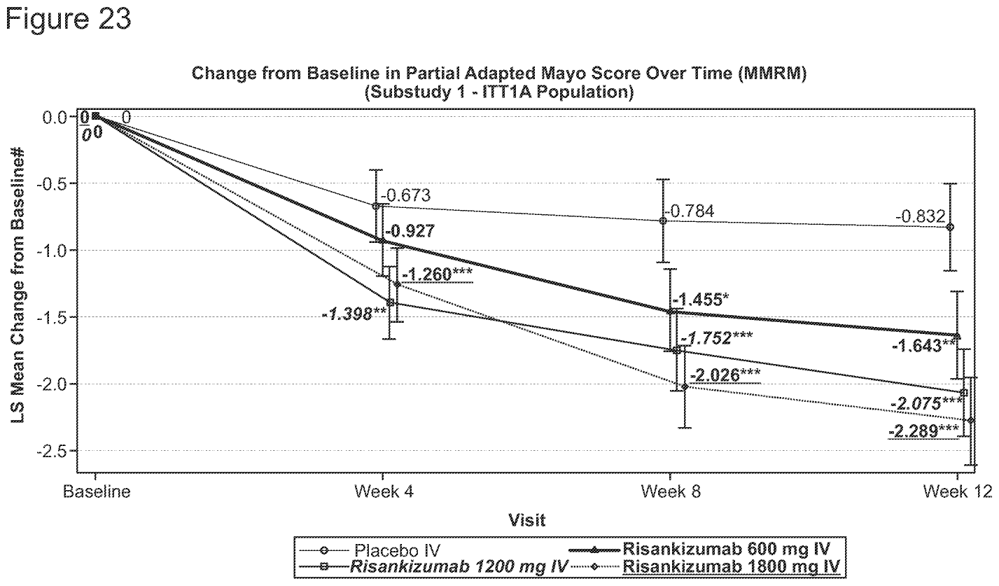

Mean change from baseline with 90% confidence intervals in partial adapted mayo score over time (FIG. 23) is presented.

4.0 Safety

4.1 Adverse Events

The overall incidences of AEs, serious AEs, severe AEs and AEs leading to discontinuation were generally balanced across treatment groups.

There was no exposure-dependent worsening in AEs, SAEs, infections, or serious infections.

Among all risankizumab treated subjects, AEs reported by >=5% of subjects were nasopharyngitis (6.1%) and headache (5%), with similar rates to placebo.

All 5 serious infections were reported in subjects treated with risankizumab. There was no trend to the types of serious infections.

No events of death, active tuberculosis, malignancies, adjudicated anaphylactic reaction or adjudicated MACE were reported in study.

4.2 Exposure-Response Analyses for Safety Parameters

There was no exposure-dependent worsening in total AEs, serious AEs, total infections or serious infections.

5.0 Pharmacokinetics and Immunogenicity

Risankizumab showed a dose-dependent increase in exposure across the three doses evaluated, indicating linear PK with no time-dependency.

Immunogenicity to risankizumab was low (1.1% and 0.5% subjects developed treatment-emergent anti-risankizumab antibodies and neutralizing antibodies by Week 12, respectively) with no dose-dependency and no impact on exposure.

7.0 Additional Descriptions and Results

7.1 Pharmacokinetics and Immunogenicity Results

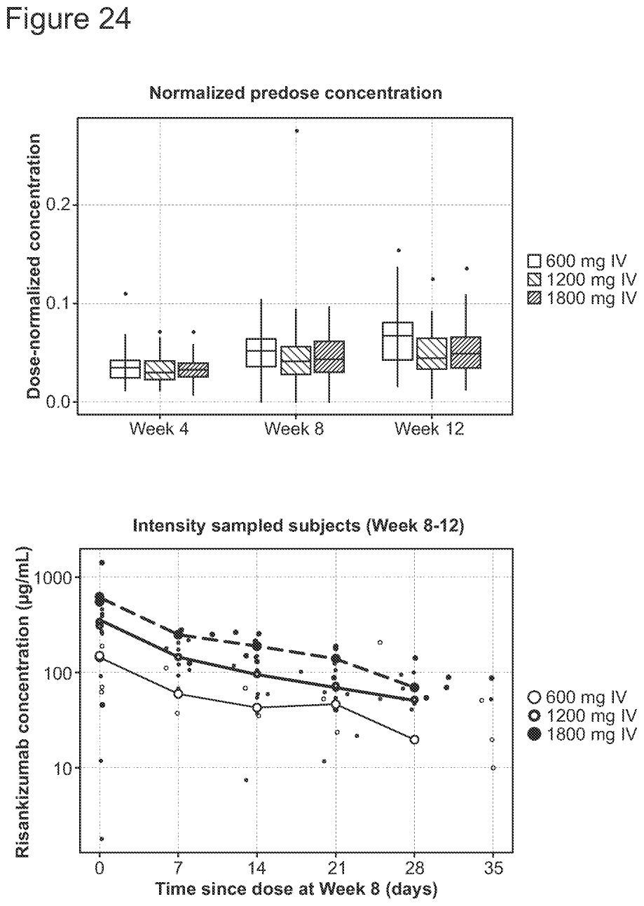

7.1.1 Pharmacokinetics Results are Shown in FIG. 24.

7.1.2 Immunogenicity Results are Shown in Table 8.

TABLE 8
Immunogenicity

| | | |
|---|---|---|
| | Treatment- | Treatment- |
| | emergent ADA | emergent nAb |
| Group | % (ADA+/Total) | % (nAb+/Total) |

| | | | | |
|---|---|---|---|---|
| Placebo IV | 0 | (0) | 0 | (0) |
| 600 mg IV | 1.6% | (1/61) | 0 | (0) |
| 1200 mg IV | 1.6% | (1/61) | 1.6% | (1/61) |
| 1800 mg IV | 0 | (0) | 0 | (0) |
| Risankizumab Total | 1.1% | (2/180) | 0.5% | (1/180) |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A method for inducing remission of moderately to severely active ulcerative colitis (UC) in an adult patient suffering from UC, comprising intravenously administering to the patient three induction doses of risankizumab at four week intervals, wherein the induction doses each comprises 600 mg, 1200 mg, or 1800 mg of risankizumab, and further wherein the patient achieves remission of the ulcerative colitis at 4 weeks, 8 weeks, or 12 weeks following the administration of the first induction dose.

2. The method of claim 1, wherein the induction dose is 600 mg.

3. The method of claim 1, wherein the induction dose is 1200 mg.

4. The method of claim 1, wherein the induction dose is 1800 mg.

5. The method of any one of claims 1-4, wherein the patient has an Adapted Mayo score of 5 to 9 points and an endoscopic subscore of 2 to 3 before the administration of the at least one induction dose of risankizumab.

6. The method of any one of claims 1-5, wherein the patient achieves, at 4 weeks, 8 weeks, or 12 weeks following the administration of the first induction dose, one or more of the endpoints selected from the group consisting of:
(1) clinical remission per Adapted Mayo: a stool frequency subscore (SFS) ≤1 and not greater than baseline, a rectal bleeding subscore (RBS)=0, and an endoscopic subscore ≤1;
(2) clinical response per Adapted Mayo: a decrease from baseline in the Adapted Mayo score ≥2 points and ≥30% from baseline, plus a decrease in a rectal bleeding subscore (RBS) ≥1 or an absolute RBS ≤1;
(3) clinical response per partial Adapted Mayo: a decrease from baseline in the Adapted Mayo score ≥1 points and ≥30% from baseline, plus a decrease in a RBS ≥1 or an absolute RBS ≤1;
(4) clinical remission per Full Mayo: Full Mayo score ≤2 with no subscore >1;
(5) endoscopic improvement: an endoscopy subscore of 0 or 1;
(6) endoscopic remission: an endoscopy subscore=0;
(7) histologic remission: a Geboes score <2; and
(8) mucosal healing: endoscopic and histologic remission.

7. The method of any one of claims 1-6, wherein the patient has intolerance or an inadequate response to one or more biologic therapies for ulcerative colitis.

8. The method of claim 7, wherein the biologic therapies comprise infliximab, adalimumab, golimumab, or vedolizumab.

9. The method of any one of claims 1-8, further comprising
(a) subcutaneously administering to the patient a first maintenance dose of risankizumab four weeks after the third induction dose is administered, and
(b) subcutaneously administering additional maintenance doses to the patient at eight week intervals after the first maintenance dose is administered, where both the first maintenance dose and the additional maintenance doses each comprises 180 mg or 360 mg of risankizumab.

10. The method of claim 9, wherein both the first maintenance dose and the additional maintenance dose are 180 mg of risankizumab.

11. The method of claim 9, wherein both the first maintenance dose and the additional maintenance dose are 360 mg of risankizumab.

12. A method for inducing remission of moderately to severely active Crohn's disease (CD) in an adult patient suffering from CD, comprising:
(a) intravenously administering to the patient three 600 mg induction doses of risankizumab at four week intervals; and
(b) subcutaneously administering to the patient a first maintenance dose of risankizumab four weeks after the third induction dose is administered; and
(c) subcutaneously administering at least one additional maintenance dose is administered at eight week intervals,
wherein the patient achieves remission of CD at 4 week, 8 weeks, 12 weeks, 24 weeks, or 52 weeks following the administration of the first induction dose.

13. The method of claim 12, wherein the patient has:
(1) average daily stool frequency (SF) score ≥4 and/or average daily abdominal pain (AP) score ≥2; or
(2) Simple Endoscopic Score for CD (SES-CD) ≥3,
before the administration of the first induction dose of risankizumab.

14. The method of claim 12 or 13, wherein the patient has Crohn's disease activity index (CDAI) score 220-450 before the administration of the first induction dose of risankizumab.

15. The method of any one of claims 12-14, wherein the patient achieves, at 4 week, 8 weeks, 12 weeks, 24 weeks, or 52 weeks following the administration of the first induction dose, one or more endpoints selected from the group consisting of:
(1) clinical remission: average daily SF [symbol] 2.8 and not worse than Baseline, and average daily AP score [symbol] 1 and not worse than Baseline;
(2) enhanced clinical response: [symbol] 60% decrease in average daily SF and/or [symbol] 35% decrease in average daily AP score and both not worse than Baseline, and/or clinical remission;
(3) clinical response: [symbol] 30% decrease in average daily SF and/or [symbol] 30% decrease in average daily AP score;
(4) endoscopic response: decrease in SES-CD >50% from Baseline, or for patients with isolated ileal disease and a Baseline SES-CD of 4, at least a 2 point reduction from Baseline;
(5) ulcer-free endoscopy: SES-CD ulcerated surface subscore of 0 in patients with SES-CD ulcerated surface subscore [symbol] 1 at Baseline;
(6) endoscopic remission: SES-CD [symbol] 4 and at least a 2 point reduction versus baseline and no subscore greater than 1 in any individual variable;
(7) Deep remission: clinical remission and endoscopic remission;
(8) CDAI clinical response: reduction of CDAI [symbol] 100 points from baseline;
(9) CDAI clinical remission: CDAI <150;
(10) steroid-free clinical remission at week 52 by CDAI: CDAI <150;
(11) steroid-free clinical remission at week 52 by SF/APS: average daily SF [symbol] 2.8 and not worse than baseline and average daily APS [symbol] 1 and not worse than baseline;
(12) steroid-free endoscopic remission at week 52: SES-CD [symbol] 4 and [symbol] 2-point reduction vs baseline and no subscore >1 in any individual variable; and
(13) steroid-free endoscopic response at week 52: >50% decrease from baseline in SES-CD (or [symbol] 2-point reduction from baseline for patients with isolated ileal disease and baseline SES-CD of 4).

16. The method of any one of claims 12-15, wherein the patient has intolerance or an inadequate response to one or more of anti-TNF, anti-integrin, or anti-p40 biologics for CD.

17. The method of claim 16, wherein the anti-TNF or anti-integrin biologic for CD comprises infliximab, adalimumab, certolizumab, vedolizumab, ustekinumab and/or natalizumab.

18. The method of any one of claims 12-17, wherein the patient has an inadequate response or intolerance to one or more conventional therapies selected from the group consisting of aminosalicylates, oral locally acting steroids, systemic corticosteroids, and immunomodulators.

19. The method of any one of claims 12-18, wherein both the first maintenance dose and the additional maintenance dose are 180 mg of risankizumab.

20. The method of any one of claims 12-18, wherein both the first maintenance dose and the additional maintenance dose are 360 mg of risankizumab.


SEQUENCE LISTING (10 sequences)

SEQ ID NO: 1 - protein, 11 aa, Artificial Sequence
  Synthetic Polypeptide
KASRDVAIAVA

SEQ ID NO: 2 - protein, 7 aa, Artificial Sequence
  Synthetic Polypeptide
WASTRHT

SEQ ID NO: 3 - protein, 9 aa, Artificial Sequence
  Synthetic Polypeptide
HQYSSYPFT

SEQ ID NO: 4 - protein, 10 aa, Artificial Sequence
  Synthetic Polypeptide
GYTFTDQTIH

SEQ ID NO: 5 - protein, 17 aa, Artificial Sequence
  Synthetic Polypeptide
YIYPRDDSPKYNENFKG

SEQ ID NO: 6 - protein, 11 aa, Artificial Sequence
  Synthetic Polypeptide
PDRSGYAWFIY

SEQ ID NO: 7 - protein, 107 aa, Artificial Sequence
  Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIK

SEQ ID NO: 8 - protein, 120 aa, Artificial Sequence
  Synthetic Polypeptide
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSS

SEQ ID NO: 9 - protein, 214 aa, Artificial Sequence
  Synthetic Poypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 449 aa, Artificial Sequence
  Synthetic Polypeptide
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG